(19) United States
(12) Patent Application Publication
(10) Pub. No.: US 2026/0263607 A1
(43) Pub. Date: Sep. 10, 2026

(54) INHIBITION OF MYC USING HIGH-DENSITY BRUSH POLYMERS

(71) Applicants: Northwestern University, Evanston, IL (US); Grove Biopharma, Inc., Chicago, IL (US)

(72) Inventors: Nathan C. GIANNESCHI, Evanston, IL (US); Max Mu WANG, Evanston, IL (US); Sarki A. ABDULKADIR, Evanston, IL (US); Mihai Ioan TRUICA, Evanston, IL (US); Paul A. BERTIN, Chicago, IL (US)

(73) Assignees: Northwestern University, Evanston, IL (US); Grove Biopharma, Inc., Chicago, IL (US)

(21) Appl. No.: 18/847,085

(22) PCT Filed: Mar. 14, 2023
(86) PCT No.: PCT/US2023/064292

§ 371 Date: Sep. 13, 2024

Related U.S. Application Data
(60) Provisional application No. 63/319,639, filed on Mar. 14, 2022.

(51) Int. Cl. A61K 47/58; A61K 9/00; A61K 38/00; A61K 47/64; A61P 35/00; C07K 7/08; C07K 14/00

(52) U.S. Cl. CPC A61K 47/58; A61K 9/0019; A61K 47/645; A61P 35/00; C07K 7/08; C07K 14/001; A61K 38/00; C07K 2319/09

(57) ABSTRACT

Disclosed are protein-like polymers and uses thereof. In an aspect, the invention provides brush protein-like polymers that address challenges associated with conventional administration of free therapeutic peptides. The protein-like polymers generally comprise a polymer of formula (FX1). The polymer of formula (FX1) in some aspects comprises a peptide having similarity or homology to a Myc binding peptide sequence. The brush protein-like polymers of the invention include high-density brush polymers including brush block polymers and brush statistical polymers. In an embodiment, brush protein-like polymers of the invention exhibit proteolysis-resistant characteristics and maintain their biological function during formulation and administration.

### CROSS-REFERENCE TO RELATED APPLICATIONS

This application claims the benefit of priority to U.S. Provisional Patent Application No. 63/319,639, filed Mar. 14, 2022, which is hereby incorporated by reference in its entirety.

### STATEMENT REGARDING FEDERALLY SPONSORED RESEARCH OR DEVELOPMENT

This invention was made with government support under Award Numbers 1F30CA257519-01 and 1F30CA250196-01, both of which awarded by the Department of Health and Human Services, National Institute of Health, and National Cancer Institute. The government has certain rights in the invention.

### REFERENCE TO AN ELECTRONIC SEQUENCE LISTING

The content of the electronic sequence listing (339509_15-22_WO_ST1.xml; Size: 31,448 bytes; and Date of Creation: Mar. 13, 2023) is herein incorporated by reference in its entirety.

### BACKGROUND

Of the ~20,000 unique proteins encoded by the human genome, only a small subset are currently able to be modulated via traditional pharmacological approaches. Included in the list of “undruggable” targets are proteins involved in a myriad of different disease states, such as transcription factors, intrinsically disordered proteins (IDPs), and various protein-protein interactions.

Small molecule drug development relies on the identification of well-defined binding pockets in the active site of proteins where compounds can be docked to affect target activity. This limitation has precluded the successful modulation of various protein-protein interactions, most of which occur along smooth, featureless interfaces. To overcome this, bifunctional small molecule PROteolysis Targeting Chimeras (PROTACs) have been developed which direct cellular degradation machinery to proteins of interest through the use of distinct domains, one which binds to the target protein and the other engages with E3 ligases to ubiquitinate and ultimately degrade the target. For example, see the PROTACs described in Goa et al., PROTAC Technology: Opportunities and Challenges. ACS Medicinal Chemistry Letters 11, 237-240 (2020) and Qi et al., PROTAC: An Effective Targeted Protein Degradation Strategy for Cancer Therapy. Front Pharmacol 12, 692574 (2021), both references are hereby incorporated by reference. However, this approach has several limitations, namely (1) allosteric docking sites are still required and (2) incorporation of more than two distinct domains is unfeasible. Accordingly, there remains a need for therapeutics targeting “undruggable” targets, such as protein-protein interactions.

One particular area of interest is in developing therapeutics targeting the Myc family of transcription factors. Myc is known to play critical roles in tumorigenesis and is dysregulated in >70% of human cancers. Heterodimerization of Myc and Max is known to be required for oncogenic transformation, yet the development of small molecule inhibitors has been hampered due to the lack of suitable binding pockets. Myc inhibitory peptides derived from the first helix (H1) of the bHLH-LZ region have been synthesized in efforts to address this limitation. However, poor pharmacokinetic profiles have precluded their clinical translation. Thus, there remains a need for therapeutics targeting the interaction between Myc and Max.

The technology described herein is intended to expand the armamentarium of compounds able to affect the activity of therapeutically relevant targets by introducing a modular proteomimetic platform technology designed to carry distinct peptide side chains. For example, disclosed herein are Heterofunctional polYmeric DegRading Chimeras (HYDRACs), which build upon lessons leamed from PROTACs and are designed to multiplex different protein binders and degradation inducing sequences. The present technology, such as platforms incorporating HYDRAC arrangements, takes advantage of certain differentiating factors which are believed to allow for improved properties including resistance to proteolysis, high levels of cell uptake, expanded interactions with featureless interfaces, and maintained bioactivity of side chains.

### SUMMARY OF THE INVENTION

In an aspect, the invention provides a polymer comprising a first repeating unit comprising a first polymer backbone group directly or indirectly covalently linked to a first functional sidechain comprising a Myc binding peptide.

In other aspects, the present invention provides a polymer comprising: a first repeating unit comprising a first polymer backbone group directly or indirectly covalently linked to a first functional sidechain comprising a Myc binding peptide; and a second repeating unit comprising a second polymer backbone group directly or indirectly covalently linked to a second functional sidechain comprising a degrader agent.

In other aspects, the invention provides a polymer comprising: a first repeating unit comprising a first polymer backbone group directly or indirectly covalently linked to a first functional sidechain comprising a Myc binding peptide; a second repeating unit comprising a second polymer backbone group directly or indirectly covalently linked to a second functional sidechain comprising a degrader agent; and a third repeating unit comprising a third polymer backbone group directly or indirectly covalently linked to a third functional sidechain comprising a nuclear localization peptide.

The present invention further includes a pharmaceutical composition comprising any one of the polymers described herein and a pharmaceutically acceptable excipient.

Further disclosed herein is a method of suppressing transcriptional expression of a target gene in a cell comprising: contacting the cell with an effective amount of any one of the polymers or pharmaceutical compositions described herein; wherein the contacting results in the suppressing transcriptional expression of the target gene in the cell.

The present invention further includes a method of targeting a nuclear localized protein in a cell comprising: introducing any one of the polymers or the pharmaceutical compositions disclosed herein to a cell; wherein the introducing results in at least a portion of the polymer or the pharmaceutical composition to bind with at least a portion of the nuclear localized protein; thereby targeting the nuclear localized protein in a cell.

In some aspects, the invention provides a method of treating or managing a condition of a subject comprising: administering to the subject a therapeutically effective amount of any one of the polymers or the pharmaceutical compositions disclosed herein; wherein the administering results in the treating or managing of the condition of the subject.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1F: Design and synthesis of Heterofunctional Protein-like Polymers (PLPs). FIG. 1A: Summary schematic of PLP structures containing side chains with different/synergistic functionalities. Note: representative sequences shown, nonexhaustive. FIG. 1B: Representative in silico model of a generalized PLP at T=0 ns (left) and T=40 ns (right) in aqueous solution, highlighting collapse into a globular architecture. FIGS. 1C-1D: Representative electrospray ionization (ESI) mass spectrometry (FIG. 1C) and high-performance liquid chromatography (HPLC) (FIG. 1D) of purified monomer species. FIGS. 1E-1F: Polymerization reaction scheme (FIG. 1E) and polymerization kinetics of PLPs with a degree of polymerization of 10 (n=10) shown in FIG. 1F.

FIGS. 2A-2F: Evaluation of toxicity in PC3 cancer cell line. FIG. 2A: Example schematic of polymerization setup. FIG. 2B: Non-exhaustive table of polymers created listing equivalents of monomer added to the reaction relative to catalyst, the theoretical DP, as well as experimentally determined DP. The notation of “s” indicates the randomly scrambled peptide sequence of an H1 was used; block: block copolymer; and stat: statistical copolymer. The subscripts denote the DP for the respective peptides FIG. 2C: Sample SDS-PAGE gels showing apparent molecular weight. Numbers above each lane correspond to the tested PLP composition as represented in the table in FIG. 2B. FIGS. 2D-2F: Dose response toxicity and IC₅₀ of unpolymerized HI peptide (FIG. 2D) block and random copolymers (FIG. 2E) and scrambled controls (FIG. 2F) in PC-3 prostate cancer cells following treatment for 72 hours. The table in FIG. 2E represents IC₅₀ values for various PLP compositions. Curves are four-parameter variable slope dose-response fits. Data depict mean±s.d. Numbers in the legend of each graph in FIGS. 2E-2F correspond to the tested PLP composition as represented in the table in FIG. 2B.

FIGS. 3A-3D: Subcellular localization of PLPs in A549 cells. FIG. 3A: depicts an exemplary Cy5.5-labled PLP structure. FIG. 3B: Representative confocal images of A549 cells treated for either 2 or 6 hours with indicated PLP compositions at 5 μM Cy5.5. Numbers above each image correspond to the tested PLP composition as represented in the table in FIG. 3C. Blue: nucleus. Red: Cy5.5-labeled PLPs. FIG. 3C: SDS-PAGE quantification of apparent molecular weight and Cy5.5 standard curve. FIG. 3D: Representative confocal images of cells treated for 2 hours with either RRRG homopolymers and scrambled copolymers (i.e., non-Myc targeted). Numbers above each image correspond to the tested PLP composition as represented in the table in FIG. 3C.

FIGS. 4A-4C: Incorporation of RRRG moiety reduces Myc protein levels. FIGS. 4A-4B: Western blot analysis of Myc protein levels following treatment with PLPs containing Myc-targeting sequence, H1, copolymerized with either the nuclear localization sequence M1 or RRRG at indicate concentrations and incubation times. Quantification of bands shown in FIG. 4B. FIG. 4C: Chemical structure of compound (left) showing dose response decrease in Myc protein levels over 24 hours as measured by western blot. 975: small molecule Myc inhibitor used as positive control.

FIGS. 5A-5B: Myc-targeted PLPs elicit on-target effects and are capable of pulling down Myc protein. FIG. 5A: Dose response toxicity in PC-3 and PC-12 cells treated with Myc HYDRAC for 72 h and cell viability quantified by CTGlo. IC₅₀ values listed. FIG. 5B: Pull down assay of biotin-terminated PLPs. Second 5.2b column (far right) denotes a separate synthetic batch. Data depict mean±s.d.

FIG. 6A-6B: Overview of MYC regulation of transcription (FIG. 6A) and relevant Myc-Max dimer inhibition sites and resulting impact of inhibition (FIG. 6B).

FIG. 7: Gel Permeation Chromatography (GPC) results for a H1-PLP having an average ~5 degrees of polymerization (DP: 4.3). The H1 sequence incorporated into the PLP comprises SEQ ID 1: NELKRAFAALRDQI. PDI means polydispersity index, DP means degrees of polymerization, and Mn means the measured experimental number average molecular weight.

FIGS. 8A-8B: PC-3 toxicity results from two metabolic assays (CCK8, FIG. 8A & CTGlo, FIG. 8B) with a H1-PLP homopolymer having an average ~5 DPs.

FIGS. 9A-9B: Comparison of GPC characterizations of Hi-Mi PLPs (FIG. 9A) with H1 PLP homopolymers (FIG. 9B). PDI means polydispersity index, M_{Theoretical} means theoretical number average molecular weight, DP means degrees of polymerization, and Mn means the measured experimental number average molecular weight.

FIGS. 10A-10B: Cell proliferation data, including IC₅₀ values, normalized to control in order to characterize heterofunctional PLPs having varying H1:M1 ratios (5:0, 5:1, and 1:1, FIG. 10A) and (15:0, 15:1, and 3:1, FIG. 10B).

FIGS. 11A-11B: PC-3 cellular toxicity data from two independent experiments (1: FIG. 11A and 2: FIG. 11B) depicting little to no impact on PC-3 viability after treatment with M1 homopolymers and peptide controls (gp100).

FIG. 12: Comparison of cellular proliferation data from free peptide (Free H1) and monomer (Nor-H1 & Nor-Mi) controls.

FIG. 13: Confocal microscopy images depicting nuclear uptake of Cy5.5-tagged PLPs after 1 hour incubation. Examples of PLP presence are denoted by white lines in the images.

FIGS. 14A-14B: Comparison of relative cellular toxicity between random H1-M1 copolymers (FIG. 14A) and block H1-M1 copolymers (FIG. 14B).

FIGS. 15A-15B: GPC graphs comparing heterofunctional PLPs with VHL as degrader agent (FIG. 15A) and RRRG as degrader agent (FIG. 15B). PDI means polydispersity index, M_{Theoretical} means theoretical number average molecular weight, and Mn means the measured experimental number average molecular weight.

FIGS. 16A-16B: PC-3 toxicity data when treated with PLPs incorporating VHL, with (FIG. 16A) or without (FIG. 16B) nuclear localization agent.

FIGS. 17A-17B: Cellular proliferation data depicting possible hook effect with H1-MI-RRRG (FIG. 17A) and H1-RRRG (FIG. 17B) PLPs.

FIGS. 18A-18B: Western blot results (FIG. 18A) from pull-down biotin assay after treatment with 10 μM of a small molecule Myc inhibitor (975) and 20 μM of varying ratios of H1-M1-RRRG PLPs and a summary of the data normalized to Myc level (FIG. 18B).

FIG. 19: Additional PC-3 viability data comparing copolymers. Nor-H1: H1 monomer, MWB-060: 5H1+5M1, MWB-058: 5H1+5RRRG, and MWB-057: 5H1+5M1 +5RRRG.

FIGS. 20A-20D: Direct PC-3 viability data, including IC₅₀ values, of comparison between RRRG copolymers and VHL copolymers, including unpolymerized peptides (FIG. 20A), polymerized PLPs (FIG. 20B), and free RRRG normalized to control (FIG. 20C). FIG. 20D provides a summary of viability data from two biological replicates comparing random/statistical (stat) PLP compositions and block PLP compositions. MWB-077: H1₆-stat-RRRG₅, MWB-076: H1₆-block-RRRG5, MWB-071: 5H1+5 M1, MWB-075: H1₆-stat-M1₅, MWB-074: H1₆-block-M1₅, MWB-062: RRRG₁₁ homopolymer, and MWB-073: H1₆ homopolymer.

FIGS. 21A-21B: Summary of results from Ebox luciferase inhibition assays evaluating HYDRAC PLP composition candidates for optimization. MYCi975: small molecule Myc inhibitor, 77: H1₆-stat-RRRG5, 62: RRRG₁₁ homopolymer.

FIG. 22: The CD spectrum results for a H1-RRRG PLP, tested at increasing concentrations of the PLP.

FIG. 23: The CD spectrum results for a PLP having a scrambled H1 sequence and RRRG degrader agent to evaluate binding ability with the bHlH region of c-Myc.

FIG. 24: The CD spectrum results for a PLP having a scrambled H1 sequence and RRRG degrader agent to evaluate Max binding activity.

FIG. 25: CD spectrum results for a H1 homopolymer (1 μM PLP), c-Myc (5 μM bHlH), the arithmetic sum of H1 homopolymer and c-Myc (Additive PLP+bHlH), and the spectrum of a mixture of H1 homopolymer and c-Myc (1 μM PLP plus 5 μM bHlH).

FIGS. 26A-26B: Summary of hemocompatibility results. Hemolytic assay (FIG. 26A) and ACT assay (FIG. 26B) after incubating cells with a PLP having a H1 sequence and RRRG degrader agent. X's in FIG. 26A (top) and X's in FIG. 26B (top) denote no signal.

FIGS. 27A-27B: PC-3 viability evaluation of unpolymerized VHL (FIG. 27A) and GPC results for polymerized H15-stat-VHLs PLP (FIG. 27B).

FIGS. 28A-28G: Design and validation of Myc targeted HYRACs. FIG. 28A: Example of a HYDRAC compound consisting of two distinct peptide side-chain domains, a protein-targeting ligand and a proteosome recruiting degron. FIG. 28B: Representation of HYDRAC in an aqueous environments. FIG. 28C: Michaelis-Menten plots of indicated polymer compositions in 0.1 μM chymotrypsin with cleavage rates of H1 monitored via HPLC. HYDRAC: H1-RRRG copolymer. FIGS. 28D-28E: Far-ultraviolet (UV) CD spectra of c-Myc (FIG. 28D) (5 μM bHlH) or Max (FIG. 28E) (5 μM Max), H1-RRRG PLP (1 μM PLP), the arithmetic sum of H1-RRRG PLP and Myc (Additive PLP+bHlH in FIG. 28D), and the spectrum of a mixture of HYDRAC and either bHLH domain (1 μM PLP plus 5 μM bHiH in FIG. 28D) or Max (1 μM PLP plus 5 μM Max in FIG. 28E) at a 5 to 1 molar ratio recorded at 20° C. FIG. 28F: Thermal denaturation of mixtures described in FIG. 28D. FIG. 28G: Target engagement of H1-containing HYDRACs. PC-3 cell lysates were treated with DMSO vehicle or indicated biotin-terminated polymer compositions at 5 μM for H-R (H1-RRRG copolymer) and 10 μM for all other compositions for 2 h, after which HYDRAC-labeled proteins underwent streptavidin pulldown, elution, separation by SDS-PAGE and blotting for Myc and GAPDH. Input lysate and pulldown lanes are shown. Representative blots from n=3 independent experiments are shown. I: Input lysate; PD: Pull down; H: biotin-terminated H1 homopolymer; sH: biotin-terminated scrambled H1 sequence; H-R: biotin-terminated H1-RRRG copolymer; sH-R: biotin-terminated scrambled H1 sequence-RRRG copolymer. Mean±SD are shown in FIG. 28C.

FIGS. 29A-29F: Myc-HYDRACs localization and targeted transcriptional regulation in PC-3 cells. FIG. 29A: Representative Cy5.5-labled polymer composition. FIG. 29B: Flow cytometry results after PC-3 cells were treated with Cy5.5-labeled polymer compositions. FIG. 29C: Confocal microscopy showing PC-3 cells after treatment with 0.5 M of Myc-HYDRAC-Cy5.5 (red) for 2 h stained with WGA-488 (green) and Hoechst 33342 (blue), fixed, and imaged. FIG. 29D: Pharmacological and thermal probes of uptake pathways showing summary data from n=3 independent experiments where PC-3 and A549 cells were pretreated with inhibitors panel. FIG. 29E: GSEA comparing gene expression profiles of vehicle versus Myc HYDRAC-treated PC-3 cells. Normalized enrichment scores (NES) and p values of top gene sets are listed. FIG. 29F: Representative plot of Hallmark Myc signatures enriched in vehicle samples compared to MYC HYDRAC treatment.

FIGS. 30A-30G. Evaluation of whether toxicity following HYDRAC treatment is composition- and/or Myc-dependent. FIGS. 30A-30D: Differential cell toxicity following treatment with indicated polymer compositions. Representative dose-response plots of PC-3 (FIG. 30A), A549 (FIG. 30B) or MycCaP (FIG. 30C) cells following 72 h treatment as quantified by CTGlo. Average IC₅₀ values fromn=3 independent biological experiments summarized in FIG. 30D. FIG. 30E: Annexin V/PI staining of PC3 cells treated for 24 h with Myc HYDRAC analyzed by flow cytometry and split into late (Annexin V-FITC⁺/PI⁺) and early (Annexin V-FITC⁺/PI⁺) stage apoptosis. FIG. 30F: Dose response curves of PC3 cells treated for 72 h with Myc HYDRACs consisting of different targeting to degron ratios. FIG. 30G: A549 cells were incubated with 10 or 20 μM of indicated polymer compositions and cell counts normalized to the seeding amount monitored over time. Data depict mean SD.

FIGS. 31A-31H: Characterizing the mechanism of HYDRAC induced Myc degradation. FIG. 31A: Western blot analysis of endogenous Myc protein in PC-3 cells treated for 24 hours with either HYDRAC (H-R) PLP or scramble control (sH-R). FIG. 31B: Western blot analysis of endogenous Myc protein in PC3 cells treated for 24 hours with H1 homopolymer (H) FIG. 31C: Western blot analysis of exogenous MYC T58A (endogenous WT Myc visible as faint band below MYC T58A) protein, constitutively expressed after stable transfection in PC3 cells, after treatment with MYCi975 or H—R at the indicated concentrations. FIG. 31D: Western blot analysis of endogenous MYC protein levels in PC-3 cells treated with H-R for 24 hours, after which drug is lifted from the media and samples collected at the indicated time points (wash). FIG. 31E: Western blot analysis of endogenous Myc protein levels in PC-3 cells treated for 24 hours with a co-mixture of H and R, as opposed to the H-R PLP and appropriate controls. All compounds at 10 μM except for H-R at 5 μM FIG. 31F: Western blot analysis of endogenous Myc protein in PC-3 cells after H-R treatment for 24 hours at 5 μM in the presence or absence of MLN4924. FIG. 31G: Western blot analysis of endogenous Myc protein in PC-3 cells treated with H-R for 24 hours at two different concentrations (5 and 10 μM) in the presence and absence of MG132. FIG. 31H: TMT-based whole proteasome quantification of PLP treatment. Data shown n=3 biologically independent samples per group.

FIGS. 32A-32C: Assessment of Myc HYDRACs in vivo. FIG. 32A: Average tumor volumes of MycCaP allografts after treatment with vehicle control or HYDRAC at 25 mg/kg dose via intraperitoneal injection. **: P<0.01. FIG. 32B: Representative images of Ki67 marker of proliferation levels assessed by IF and cleaved Caspase-3 levels assessed by IHC in the tumor tissue from the study in FIG. 32A. (scale bar, 100 μM). FIG. 32C: Luc-MV4-11 tumor bearing mice injected once with Cy5.5-labeled HYDRAC and tracked by IVIS over 72 h.

FIGS. 33A-33C: Generalizability of the HYDRAC platform. FIG. 33A: Structures of four MYC-targeting HYDRACs incorporating three different E3 ligase recruiting peptides or small molecule. Representative western blot (FIG. 33B) and quantification (FIG. 33C) of endogenous MYC protein levels after treatment of PC-3 cells for 24 hours with indicated polymer compositions and concentrations. n=3 biologically independent samples per group. Data depict mean±s.d. P-values in FIG. 33C determined by t-test compared to vehicle-treated controls. *: P<0.05, **: P<0.01, ns: not significant.

### STATEMENTS REGARDING CHEMICAL COMPOUNDS AND NOMENCLATURE

The following abbreviations are used herein: SPPS refers to solid phase peptide synthesis; ROMP refers to ring-opening metathesis polymerization; RAFT refers to reversible addition fragmentation chain transfer polymerization; DMF refers to dimethylformamide; TFA refers to trifluoroacetic acid; TIPS refers to triisopropyl silane; DTT refers to dithiothreitol; LJ refers to Lennard-Jones; RP-HPLC refers to reverse-phase high performance liquid chromatography; ESI-MS refers to electrospray ionization mass spectrometry; NMR refers to nuclear magnetic resonance spectrometry; MALDI-MS refers to matrix-assisted laser desorption/ionization mass spectrometry; SEC-MALS refers to size-exclusion chromatography coupled with multiangle light scattering; GPC refers to gel permeation chromatography; SDS-PAGE refers to sodium dodecyl sulfate-polyacrylamide gel electrophoresis; CD refers to circular dichroism; SAXS refers to Small-angle X-ray scattering; ARE refers to antioxidant response element; BSA refers to bovine serum albumin; tBHQ refers to tert-Butylhydroquinone; PLP refers to protein-like polymer; NP refers to nanoparticle; PDI refers to polydispersity index; MW refers to molecular weight; and DP refers to degree of polymerization.

In an embodiment, a peptide, a polymer, or a composition (e.g., formulation) of the invention is isolated or purified. In an embodiment, an isolated or purified peptide, polymer, or composition (e.g., formulation) is at least partially isolated or purified as would be understood in the art. In an embodiment, the peptide, polymer, or composition (e.g., formulation) of the invention has a chemical purity of at least 95%, optionally for some applications at least 99%, optionally for some applications at least 99.9%, optionally for some applications at least 99.99%, and optionally for some applications at least 99.999% pure. The invention includes isolated and purified compositions of any of the brush polymers described herein including the peptide brush and block copolymers and brush and brush block copolymers having one or more side chains comprising the peptide analogues, derivative, variants or fragments.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and a high molecular weight (e.g., greater than or equal to 1 kDa, in some embodiments greater than or equal to 5 kDa or greater than or equal to 50 kDa).

Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits (e.g., 3 or more monomer subunits, 4 or more monomer subunits, 5 or more monomer subunits, or 6 or more monomer subunits), and include random, block, brush, brush block, alternating, segmented, grafted, tapered and other architectures. In some embodiments, copolymers of the invention comprise from 2 to 10 different monomer subunits. Useful polymers include organic polymers that may be in amorphous, semi-amorphous, crystalline or semi-crystalline states. Cross linked polymers having linked monomer chains are useful for some applications, for example linked by one or more disulfide linkages. In embodiments, the invention provides polymers comprising therapeutic agents, such as brush polymers having at least a portion of the repeating units comprising polymer side chains such as peptide side chains.

As used herein, the term “polymer segment” (e.g., first polymer segment, second polymer segment, etc.) refers to a section (e.g., portion) of the polymer comprising a particular monomer or arrangement of monomers. A polymer segment can be a homopolymer or a copolymer. In embodiments where a polymer segment is a copolymer, the copolymer can exist in any suitable arrangement of monomers (e.g., random, block, brush, brush block, alternating, segmented, grafted, tapered, statistical and other architectures). In some embodiments, the polymer segments are homopolymers, random copolymers, statistical copolymers, or block copolymers. Any polymer (e.g., brush polymer) described herein can have a single polymer segment or multiple polymer segments. In embodiments where the polymer has multiple polymer segments, the polymer segments can exist in any suitable arrangement (random, block, brush, brush block, alternating, segmented, grafted, tapered, statistical, and other architectures).

An “oligomer” refers to a molecule composed of repeating structural units connected by covalent chemical bonds often characterized by a number of repeating units less than that of a polymer (e.g., equal to or less than 3 repeating units) and weight average lower molecular weights (e.g., less than or equal to 1,000 Da) than polymers. Oligomers may be the polymerization product of one or more monomer precursors.

A “peptide” or “oligopeptide” herein refer to a polymer of repeating structural units connected by peptide bonds, including, for example, polypeptides. Typically, the repeating structural units of the peptide are amino acids including naturally occurring amino acids, non-naturally occurring amino acids, analogues of amino acids or any combination of these. The number of repeating structural units of a peptide, as understood in the art, are typically less than a “protein”, and thus the peptide often has a lower molecular weight than a protein. In some embodiments, a peptide has a chain length of 3 to 150 amino acids, optionally 3 to 100 amino acids, optionally 5 to 50 amino acids, and optionally 5 to 30 amino acids.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e., adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g., [A][B]), or may be provided in a selected sequence ([A][B][A][B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Statistical copolymers,” also generally known in the art as “random copolymers,” are copolymers in which the ordering of backbone groups is dictated by reaction kinetics and comprise spatially randomized units, wherein at least two chemically distinguishable polymerized monomers are randomly distributed throughout the polymer. Statistical copolymers generally are antithetical to block copolymers.

“Polymer backbone group” or “polymer backbone subunit” refers to groups that are covalently linked to make up a backbone of a polymer, such as a block copolymer. Polymer backbone groups may be linked to side chain groups, such as polymer side chain groups. Some polymer backbone groups useful in the present compositions are derived from polymerization of a monomer selected from the group consisting of a substituted or unsubstituted norbornene, olefin, cyclic olefin, norbomene anhydride, cyclooctene, cyclopentadiene, styrene, acrylamide, and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen, C₁-C₁₀ alkyl or C₅-C₁₀ aryl. In some embodiments, polymer backbones may terminate in backbone terminating groups including hydrogen, C₁-C₅ alkyl, C₃-C₅ cycloalkyl, C₅-C₈ aryl, C₅-C₈ heteroaryl, C₁-C₅ acyl. In some embodiments, polymer backbones may terminate in backbone terminating groups including hydrogen, C₁-C₃ alkyl.

“Polymer side chain group” (also sometimes referred to herein as “substituent,” e.g., with respect to R¹) refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition. A polymer side chain group may be directly or indirectly linked to the polymer back bone groups. In some embodiments, polymer side chain groups provide steric bulk and/or interactions that result in an extended polymer backbone and/or a rigid polymer backbone. Some polymer side chain groups useful in the present compositions include unsubstituted or substituted peptide groups. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen or C₁-C₅ alkyl.

As used herein, the term “responsive” refers to an agent or a peptide wherein at least a portion of its composition is capable of interacting with at least a portion of a specific molecule. For example, a responsive peptide may include an amino acid sequence corresponding to a cut-site for a specific enzyme.

As used herein, the term “nonresponsive” refers to an agent or a peptide having a composition that is not known to interact with a specific molecule.

As used herein, the term “degree of polymerization” refers to the average number of monomer units per polymer chain. For example, for certain polymers described herein, comprising B¹, B², B³ and B⁴ backbone units, the degree of polymerization would be represented by the sum total of B¹, B², B³, and B⁴ backbone units. Since the degree of polymerization can vary from polymer to polymer, the degree of polymerization is generally represented by an average.

As used herein, the term “targeting agent” refers to an agent that directs transport of a polymer to a specific region of a subject. In embodiments, the targeting agent directs transport to a disease site of a subject, an inflammation site of a subject, a tumor of a subject, a tissue of a subject, an organ or an organelle of a subject, a cell of a subject, an intracellular receptor of a subject, an extracellular receptor of a subject, a transmembrane receptor of a subject, an enzyme of a subject, a protein-protein interaction of a subject, or any combination thereof. In embodiments, the targeting agent facilitates localization and/or aggregation of the polymer at the target site. In some embodiments, the targeting agent is a targeting peptide or component or fragment thereof. In some embodiments, a targeting agent comprises a targeting peptide having a chain length of 3 to 150 amino acids, optionally of 3 to 100 amino acids, optionally 5 to 50 amino acids, optionally 5 to 20 amino acids.

The targeting peptide may be a naturally-occurring peptide, a synthetic peptide, or a purified recombinant peptide. In some aspects, the targeting peptide is a naturally-occurring fusion peptide or a synthetic fusion peptide. In aspects of the invention, the targeting peptide may be modified. In aspects, the modification may comprise a residue-specific modification, a peptide backbone modification, an N-terminal modification, a C-terminal modification, or any combination thereof. In examples, the modification may improve peptide stability, alter peptide structure, incorporate imaging and/or detection agents, improve solubility, enhance non-specific enzyme resistance, reduce steric hindrance, increase cellular penetration, improve binding affinities to targets, enhance safety, or any combination thereof. For example, a modification may include one or more of: biotin labeling, fluorescent dye labeling such as cyanine labeling, fluorescein and 7-methoxycoumarin acetic acid labeling, dansyl and/or 2,4-dinitrophenyl labeling, EDANS labeling, coumarin labeling, and/or rhodamine labeling, one or more point mutations, introduction of one or more spacers, isotopic labeling, introduction of one or more chelating agents, acetylation, amidation, methylation, palmitylation, hydroxylation, glycosylation, sulfation and sulfonation, esterification, phosphorylation, peptide stapling, lipidation, cyclization, or any combination thereof.

In some embodiments, the targeting peptide is characterized by a number average molecular weight (Mn) less than or equal to 15 kDa, optionally less than or equal to 10 kDa, optionally less than or equal to 7.5 kDa, optionally less than or equal to 5 kDa, optionally less than or equal to 2.5 kDa, or optionally less than or equal to 2 kDa. In some embodiments, the targeting peptide is characterized by a number average molecular weight of 0.5 kDa to 20 kDa, optionally of 0.5 kDa to 10 kDa, optionally of 0.5 kDa to 5 kDa, or optionally of 0.5 kDa to 2.5 kDa.

The term “therapeutic agent” as used herein refers to a class of agents capable of treating or managing a disease, illness, or other condition of a subject. In some embodiments, the therapeutic agent is a pharmaceutical or biological agent or component or fragment thereof. In an embodiment, the therapeutic agent is a therapeutic peptide or component or fragment thereof. In embodiments, the therapeutic agent may be a therapeutic peptide having a chain length of 3 to 150 amino acids, optionally of 3 to 100 amino acids, optionally 5 to 50 amino acids and optionally 5 to 20 amino acids. In some embodiments, the therapeutic agent may comprise a small molecule therapeutic. In examples, the small molecule therapeutic comprises a low molecular weight organic compound having a molecular weight of less than or equal to 2 kDa, optionally less than or equal to 1.5 kDa, or optionally less than or equal to 1 kDa.

The therapeutic peptide may be a naturally-occurring peptide, a synthetic peptide, or a purified recombinant peptide. In some aspects, the therapeutic peptide is a naturally-occurring fusion peptide or a synthetic fusion peptide. In embodiments, the therapeutic peptide may be an agonist (activator) or an antagonist (inhibitor) of enzymatic activity or function, protein activity or function, gene expression, or a combination thereof. In some embodiments, the therapeutic peptide is a reversible antagonist or a reversible agonist. In some embodiments, the therapeutic peptide is an irreversible antagonist or an irreversible agonist.

In aspects of the invention, the therapeutic peptide may be modified. In aspects, the modification may comprise a residue-specific modification, a peptide backbone modification, an N-terminal modification, a C-terminal modification, or any combination thereof. In examples, the modification may improve peptide stability, alter peptide structure, incorporate imaging and/or detection agents, improve solubility, enhance non-specific enzyme resistance, reduce steric hindrance, increase cellular penetration, improve binding affinities to targets, enhance safety, or any combination thereof. For example, a modification 20 may include one or more of: biotin labeling, fluorescent dye labeling such as cyanine labeling, fluorescein and 7-methoxycoumarin acetic acid labeling, dansyl and/or 2,4-dinitrophenyl labeling, EDANS labeling, coumarin labeling, and/or rhodamine labeling, one or more point mutations, introduction of one or more spacers, isotopic labeling, introduction of one or more chelating agents, acetylation, amidation, methylation, palmitylation, hydroxylation, glycosylation, sulfation and sulfonation, esterification, phosphorylation, peptide stapling, lipidation, cyclization, or any combination thereof.

In some embodiments, the therapeutic peptide is characterized by a number average molecular weight (Mn) less than or equal to 15 kDa, optionally less than or equal to 10 kDa, optionally less than or equal to 7.5 kDa, optionally less than or equal to 5 kDa, optionally less than or equal to 2.5 kDa, or optionally less than or equal to 2 kDa. In some embodiments, the therapeutic peptide is characterized by a number average molecular weight of 0.5 kDa to 20 kDa, optionally of 0.5 kDa to 10 kDa, optionally of 0.5 kDa to 5 kDa, or optionally of 0.5 kDa to 2.5 kDa.

“Myc” is a master transcription factor responsible for regulating essential cellular processes (proliferation, metabolism, biosynthesis, apoptosis), many of which when corrupted are recognized as hallmarks of cancer. The MYC gene is part of a family that includes MYCL and MYCN and together they are one of the members of the basic helix-loop-helix leucine zipper (bHLH-LZ) superfamily of transcription factors that bind DNA as dimers.

As used herein, the term “Myc binding peptide” refers to a targeting agent configured to selectively target a Myc protein. In some embodiments, the Myc binding peptide selectively targets a n-Myc protein or a 1-Myc protein. In some embodiments, the Myc binding peptide selectively targets a c-Myc protein. In keeping with this aspect of the invention, at least a portion of the Myc binding peptide may selectively bind to at least a portion of one or more regions of the Myc protein, including the N-terminal region, the internal region, and the C-terminal region. In examples, at least a portion of the Myc binding peptide selectively binds to at least a portion of the N-terminal region of c-Myc. At least a portion of the Myc binding peptide may selectively bind to at least a portion of the transactivation domain, the MBo sub-region, the MBI sub-region, the MBII sub-region, or any combination thereof, of the N-terminal region. In some embodiments, at least a portion of the Myc binding peptide selectively binds to at least a portion of the internal region of c-Myc. In examples, at least a portion of the Myc binding peptide selectively binds to at least a portion of the PEST domain, the nuclear localization sequence domain, the MBTIIa sub-region, the MBIIIb sub-region, the MBIV sub-region, or any combination thereof, of the internal region of c-Myc. In some embodiments, at least a portion of the Myc binding peptide selectively binds to at least a portion of the C-terminal region of c-Myc. In examples, at least a portion of the Myc binding peptide selectively binds to at least a portion of the bHLHZip domain of c-Myc.

In aspects, the Myc binding peptide is derived, at least in part, from the bHLHZip domain of c-Myc. In some aspects the Myc binding peptide comprises a sequence corresponding to one or more portions of the the helix-1 (H1) region of c-Myc. In other aspects, the Myc binding peptide comprises a sequence corresponding to one or more portions of the leucine zipper region of c-Myc. In embodiments, the Mvc binding peptide may be a naturally-occurring peptide, a synthetic peptide, or a purified recombinant peptide. In some aspects, the Myc binding peptide is a naturally-occurring modified peptide, fragment peptide or fusion peptide or a synthetic modified peptide, fragment peptide or fusion peptide. In some embodiments, the Myc binding peptide is characterized by molecular weight between 1 kDa and 10 kDa. In some embodiments, the Myc binding peptide is characterized by molecular weight between 500 Da and 2500 Da. In some embodiments, the Myc binding peptide is characterized by molecular weight between 1000 Da and 2000 Da.

In further aspects, a “Myc binding peptide” herein also refers to a therapeutic agent. For example, the Myc binding peptide may be a therapeutic peptide capable of treating or managing cancer. In further aspects, the Myc binding peptide may be capable of treating or managing c-Myc dependent cancers. In aspects wherein the Myc binding peptide selectively targets a protein-protein interaction between c-Myc and Max or a DNA-protein interaction involving c-Myc, the Myc binding peptide comprises an inhibitor. In some further aspects wherein the Myc binding peptide is an inhibitor, the Myc binding peptide prevents the dimerization of c-Myc and Max. In some aspects wherein the Myc binding peptide is an inhibitor, the Myc binding peptide prevents the interaction between c-Myc and promoters of specific target genes. In aspects, the Myc binding peptide comprises any suitable number of amino acid units so long as the peptide comprises a sequence having 75% or greater (e.g., 75% or greater, 80% or greater, 85% or greater, 90% or greater, 95% or greater, or 100%) sequence identity of SEQ ID: 1 (NELKRAFAALRDQI), SEQ ID: 2 (NELKRSFFALRDQI), or SEQ ID: 3 (NELKRSFAALRDQI). In other aspects, the Myc binding peptide comprises any suitable number of amino acid units so long as the peptide comprises a sequence having 75% or greater (e.g., 75% or greater, 80% or greater, 85% or greater, 90% or greater, 95% or greater, or 100%) sequence identity of SEQ ID: 4 (VQAEEQKLISEEDLLRKRREQLKHKLEQLRN).

As used herein, “H1” refers to a H1 Myc inhibitory peptide. In embodiments, H1 is derived from the helix 1 of the basic helix-loop-helix (bHLH) leucine zipper (LH) region of the Myc peptide. In some embodiments, H1 comprises a short 14-amino acid peptide (e.g., SEQ ID: 1 (NELKRAFAALRDQI)) that is believed to inhibit Myc-Max heterodimer formation and subsequent binding to DNA.

As used herein, a “degrader agent” or “degron” refers to a class of agents capable of directly or indirectly facilitating the regulation of protein degradation. For example, the regulation may comprise promoting degradation, inhibiting degradation, increasing the rate of degradation, and/or decreasing the rate of degradation of a protein. In embodiments, such as wherein the degrader agent is incorporated in a PLP, the degrader agent facilitates specific degradation of a targeted protein. In some aspects of the invention, the degrader agent is Ubiquitin dependent. In other aspects, the degrader agent is Ubiquitin-independent. Without subscribing to a particular theory, it is believed that in aspects wherein the degrader agent is Ubiquitin dependent, the degrader agent participates in the polyubiquitination process to target proteins, or fragments thereof, for degradation by a proteasome. In these aspects, the degrader agent may be referred to herein as a “proteasome recruiter.” In some embodiments, the degrader agent is a degrader peptide or component or fragment thereof. In embodiments, the degrader agent may be a degrader peptide having a chain length of 3 to 150 amino acids, optionally of 3 to 100 amino acids, optionally 5 to 50 amino acids, optionally 5 to 20 amino acids, and optionally 4 to 10 amino acids. In some embodiments, the degrader agent may comprise a small molecule degrader. In examples, the small molecule degrader comprises a low molecular weight organic compound having a molecular weight of less than or equal to 2 kDa, optionally less than or equal to 1.5 kDa, or optionally less than or equal to 1 kDa. In some embodiments, the degrader agent is characterized by molecular weight between 100 Da and 2000 Da. In some embodiments, the degrader agent is characterized by molecular weight between 250 Da and 1500 Da.

In aspects, the degrader agent is a therapeutic agent. For example, the degrader agent may be a therapeutic peptide and/or a small molecule therapeutic capable of treating or managing cancer. In embodiments wherein the degrader agent is a therapeutic peptide and/or a small molecule therapeutic, the degrader agent may directly or indirectly promote selective degradation of Myc protein. In some aspects wherein the degrader agent promotes the degradation of Myc protein, cell viability may decrease. In embodiments, the degrader agent comprises a proteasome-targeting chimera (“PROTAC”). In aspects, the degrader agent comprises any suitable number of amino acid units so long as the peptide comprises a sequence having 75% or greater (e.g., 75% or greater, 80% or greater, 85% or greater, 90% or greater, 95% or greater, or 100%) sequence identity of SEQ ID NO: 9 (RRRG) or SEQ ID: 10 (RRRGN). In aspects, the degrader agent comprises any suitable number of amino acid units so long as the peptide comprises a sequence having 75% or greater (e.g., 75% or greater, 80% or greater, 85% or greater, 90% or greater, 95% or greater, or 100%) sequence identity of SEQ ID 14: (ALAPYIP) or SEQ ID: 15 (ALAPYIPR). In aspects, the degrader agent comprises any suitable number of amino acid units so long as the peptide comprises a sequence having 75% or greater (e.g., 75% or greater, 80% or greater, 85% or greater, 90% or greater, 95% or greater, or 100%) sequence identity of SEQ ID 16: (LDPETGEYL). In some aspects, the degrader agent comprises a small molecule therapeutic comprising thalidomide or an analogue thereof.

As used herein, “HYDRAC” refers to Heterofunctional polYmeric DegRading Chimeras (HYDRACs). In aspects, HYDRACS are a subclass of PLPs which contain heterologous side chains with distinct functionalities, wherein one domain binds to a protein of interest and a second targets it for degradation.

As used herein, a “nuclear localization peptide” refers to a targeting agent configured to facilitate targeted transport of at least a portion of a polymer into a cell nucleus. In some embodiments, the nuclear localization peptide comprises a spacer sequence of less than or equal to 20 amino acid residues (e.g., less than or equal to 20 amino acid residues, less than or equal to 15 amino acid residues, less than or equal to 10 amino acid residues, or less than or equal to 5 amino acid residues). In aspects, the nuclear localization peptide comprises any suitable number of amino acid units so long as the peptide comprises a sequence having 75% or greater (e.g., 75% or greater, 80% or greater, 85% or greater, 90% or greater, 95% or greater, or 100%) sequence identity of SEQ ID NO: 18 (PAAKRVKLD). In some embodiments, the nuclear localization peptide is characterized by molecular weight between 500 Da and 2500 Da. In some embodiments, the nuclear localization peptide is characterized by molecular weight between 500 Da and 1500 Da.

In embodiments, L¹ and L² are linking groups, and optionally a linking group comprising a polymer grafting group. In some embodiments, L¹ and L² independently are optionally functionalized by one or more additional substituents, such as peptide substituents or substituents derived from small molecules. In some embodiments, L¹ and L² independently comprise a linking group selected from the group consisting of a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20.

As used herein, the term “brush polymer” refers to a polymer comprising repeating units each independently comprising a polymer backbone group covalently linked to at least one polymer side chain group. A brush polymer may be characterized by brush density which refers to the percentage of the repeating units comprising polymer side chain groups. Brush polymers of certain aspects are characterized by a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a density selected from the range of 75% to 100%, or optionally for some embodiments a density selected from the range of 90% to 100%. Brush polymers, such as the polymers disclosed herein (e.g., a polymer of formula (FX1)), can be prepared by any suitable methods including, “grafting from” methods, “grafting onto” methods, “grafting through” methods, or any combination thereof. Such suitable methods can include, for example, ring opening metathesis polymerization (ROMP) synthetic pathways and/or non-ROMP synthetic pathways, such as, by way of example, reversible addition fragmentation chain transfer (RAFT) polymerization, stable free radical mediated polymerization and atom transfer radical polymerization (ATRP).

As used herein, the term “peptide density” refers to the percentage of monomer units in the polymer chain which have a peptide or small molecule covalently linked thereto, and such “peptide density” can be calculated generally for all peptides or small molecules or for a specific peptide or small molecule. The percentage is based on the overall sum of monomer units in the polymer chain. For example, for certain polymers described herein, each P¹ is the polymer side chain comprising the Myc binding peptide, each P⁰ indicates the absence of the Myc binding peptide polymer side chain. Thus, the P¹ density, or percentage of first backbone monomer units comprising the therapeutic agent, may be represented by the formula:

$$\frac{{P}^{1}}{{P}^{1} + {P}^{0}} \times 100 ,$$

where each variable refers to the number of monomer units of that type in the polymer chain. Polymers of certain aspects are characterized by a peptide density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Polymers of certain aspects are characterized by a peptide density selected from the range 50% to 100%, optionally some embodiments a density selected from the range of 75% to 100%, or optionally for some embodiments a density selected from the range of 90% to 100%. In some embodiments, the brush density is equal to the peptide density.

In an aspect, the polymer side chain groups (e.g., also termed substituents herein) can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6±5 angstroms apart on the polymer backbone. In some embodiments the brush polymer has a high a brush density (e.g., greater than 70%), wherein the polymer side chain groups are spaced 5 to 20 angstroms apart on the polymer backbone.

As used herein, the term “sequence homology” or “sequence identity” means the proportion of amino acid matches between two amino acid sequences of interest in two different peptides considering the ordering of the amino acids. Matches occur when amino acids are in the same order in one peptide compared to the other peptide. When sequence homology is expressed as a percentage, e.g., 50%, the percentage denotes the fraction of matches over the length of sequence that is compared to some other sequence, considering the amino acid order. Gaps (in either of the two sequences) are permitted to maximize matching; for example, wherein gap lengths of 10 amino acids or less, optionally 5 amino acids or less, optionally 3 amino acids or less, are usually used. In other words, a sequence having 75% or greater sequence identity to an amino acid sequence with 9 amino acids can indicate that the 9 amino acid sequence can have one or two point mutations (i.e., amino acid change), one or two amino acid deletions, one or two amino acid additions, one point mutation and one amino acid deletion, or one point mutation and one amino acid addition. Even with two such amino acids being different, 7 out of 9 amino acids still match in the correct order, such that there is greater than 75% sequence identity. For clarity, the analysis of whether there sequence homology between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

As used herein, the term “amino acid composition similarity” or “amino acid similarity” means the proportion of amino acid matches between two amino acid sequences of interest in two different peptides regardless of the ordering of the amino acids. Matches occur when amino acids are present in both amino acid sequences regardless of order. When amino acid composition similarity is expressed as a percentage, e.g., 50%, the percentage denotes the fraction of matches over the length of sequence that is compared to some other sequence, regardless of amino acid order. Gaps (in either of the two sequences) are permitted to maximize matching; for example, wherein gap lengths of 5 amino acids or less, optionally 3 amino acids or less, are usually used. By way of example, if two amino acid sequences each containing ten amino acids have three amino acids in common, in any order, then there is 30% amino acid composition similarity between the sequences. For clarity, the analysis of whether there is amino acid composition similarity between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

The terms “natural protein” or “natural peptide” as used herein refer to peptides or proteins that are found in nature. Although such peptides or proteins may be able to be synthesized in a lab setting, natural peptides or proteins were originally discovered in nature, e.g., being produced by natural organisms, such as mussels.

The term “fragment” refers to a portion, but not all of, a composition or material, such as a peptide composition or material. In an embodiment, a fragment of a peptide refers to 50% or more of the sequence of amino acids, optionally 70% or more of the sequence of amino acids and optionally 90% or more of the sequence of amino acids.

“Polymer blend” refers to a mixture comprising at least one polymer, such as a brush polymer, e.g., brush block copolymer, and at least one additional component, and optionally more than one additional component. In some embodiments, for example, a polymer blend of the invention comprises a first brush copolymer and one or more addition brush polymers having a composition different than the first brush copolymer. In some embodiments, for example, a polymer blend of the invention further comprises one or more additional brush copolymers, homopolymers, copolymers, block copolymers, brush block copolymers, oligomers, solvent, small molecules (e.g., molecular weight less than 500 Da, 5 optionally less than 100 Da), or any combination of these. Poly mer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

The term “antibody mimetic” refers to an organic compound with the ability to specifically bind antigens but are not structurally related to antibodies. Typical antibody mimetics are not produced by a subject's immune system and instead are artificially produced. Additionally, antibody mimetics are generally smaller than antibodies and have greater stability. However, it will be understood that antibody mimetics may be synthetically produced to comprise specific properties depending on desired outcome, including variable size, greater stability, greater affinity, protease-resistance and improved solubility. For example, antibody mimetics include peptide aptamers, affitins, avimers, armadillo repeat proteins, designed ankryin repeat proteins (DARPins), and anticalins.

As used herein, the term “compound” can be used to refer to any of the peptides or polymers described herein. Alternatively, or additionally, the term compound can refer to any of the synthetic precursors, reagents, additives, excipients, etc. used in preparation of or formulation with the peptides or polymers described herein.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” generally refers to a compound wherein a hydrogen is replaced by another functional group, unless otherwise contradicted by context.

As is customary and well known in the art, hydrogen atoms in formulas (FX1)-(FX3) and (RU1)-(RU7) are not always explicitly shown, for example, hydrogen atoms bonded to the carbon atoms of aromatic, heteroaromatic, and alicyclic rings are not always explicitly shown in formulas (FX1)-(FX3) and (RU1)-(RU7). The structures provided herein, for example in the context of the description of formulas (FX1)-(FX3) and (RU1)-(RU7) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the phrase “charge modulating domain” refers to one or more amino acids added to the peptide sequences described herein to modulate the charge of the peptide. For example, the charge modulating domain can be a TAT sequence, a glycine-serine domain, a cationic residue domain, or a combination thereof, or optionally a glycine-serine domain, a cationic residue domain, or a combination thereof. In certain embodiments, the charge modulating domain has from 2 to 7 amino acid residues. The 2 to 7 amino acids can be added in a single block containing from 2 to 7 amino acid residues or more than one block containing from 1 to 6 amino acid residues. In preferred embodiments, the charge modulating domain is a cationic residue domain having from 2 to 7 amino acid residues 20 selected from lysine, arginine, histidine, or a combination thereof. Generally, the charge modulating domain modulates the charge of the peptide to have a net positive charge. Without wishing to be bound by any particular theory, it is believed that the net positive charge increases the cellular uptake of the peptide or polymer comprising the peptide. The overall charge of the peptide or copolymer comprising the peptide can be determined by any suitable means. For example, the overall charge can be determined by (i) structural analysis of the functional residues on the peptide sequence and their respective pKa, (ii) physical characterization by measuring the zeta potential, and/or (iii) by virtue of the material moving towards a negative pole in an electrophoresis polymer gel. In certain embodiments, the overall charge of the peptide or copolymer comprising the peptide is determined by measuring the zeta potential.

Unless otherwise specified, the term “average molecular weight,” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g., L¹, L²).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g., L¹, L²).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g., L¹, L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g., Li, L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g., L¹, L²).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃—CIO cycloalkenylene and C₃-C₅ cycloalkenylene groups, for example, as one or more linking groups (e.g., L¹, L², L³).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g., L¹, L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “poly alkoxy alkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural a-amino acid” specifically includes the side chains of the above-referenced amino acids. Peptides are comprised of two or more amino acids connected via peptide bonds. As used herein, “protected amino acids” refer to amino acids in which the amine group and/or the carboxylic acid group are protected by a temporary protecting group. For example, t-butyloxycarbonyl (Boc) and 9-fluorenlmethoxycarbonyl (Fmoc) are temporary protecting groups used in SPPS.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO—refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —CN;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, which is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L-for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “~” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g., achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). Another example of an effective amount includes a “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g., transcription factor) relative to the absence of the antagonist. An “activity increasing amount,” as used herein, refers to an amount of agonist (activator) required to increase the activity of an enzyme or protein (e.g., transcription factor) relative to the absence of the agonist. A “function disrupting amount,” as used herein, refers to the amount of antagonist (inhibitor) required to disrupt the function of an enzyme or protein (e.g., transcription factor) relative to the absence of the antagonist. A “function increasing amount,” as used herein, refers to the amount of agonist (activator) required to increase the function of an enzyme or protein (e.g., transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art. Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The expression “hydrophilic amino acid residue” refers to an amino acid group, modified amino acid group or substituted amino acid group having at least partial hydrophilic character under at least some conditions, such as in vivo conditions, including arginine, asparagine, aspartate, glutamine, glutamate, or lysine. The expression “neutral amino acid residue”, refers to an amino acid group, modified amino acid group or substituted amino acid group having at least neutral charge character under at least some conditions, such as in vivo conditions, including as histidine, proline, or tyrosine. The polymers of some embodiments comprise peptides having one or more hydrophilic amino acid residues and/or neutral amino acid residues.

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g., agonist) interaction means positively affecting (e.g., increasing) the activity or function of the protein.

The term “modulator” refers to a composition that increases or decreases the level of a target molecule or the function of a target molecule.

“Patient” “subject” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the composition of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Nanoparticles (NPs) are a type of nanocarrier (NC) capable of transporting small molecules throughout a subject, providing protection to small molecules from a surrounding environment, protecting the surrounding environment from biological activity of small molecules, and/or targeting delivery of small molecules to a specific site. NPs may be polymeric NPs, which generally have a size between the range of 1 to 1000 nm. NPs are generally categorized as nanospheres or nanocapsules. See Zielinska et al., Molecules, 25: 3731 (2020). As used herein, NPs configured to transport therapeutic agents are referred to as “drug-loaded NPs.”

### DETAILED DESCRIPTION

In the following description, numerous specific details of the polymers, polymer components, compositions, and methods of the present invention are set forth in order to provide a thorough explanation of the precise nature of the invention. It will be apparent, however, to those of skill in the art that the invention can be practiced without these specific details.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features describe with respect to one embodiment may be incorporated with other embodiments as well. By the same token, however, no single feature or features of any described or claimed embodiment should be considered essential to every embodiment of the invention, as other embodiments of the invention may omit such features.

The PLPs described herein comprise single chains comprising a hydrophobic polymer backbone having a dense display of peptide sequences and/or small molecules. It is hypothesized that PLPs fold upon themselves in aqueous solution by the hydrophobic effect, which excludes water from the synthetic hydrophobic polymer backbone, giving a globular morphology akin to that of proteins. It is believed that these features contribute to the proteolytic resistance of PLPs in vivo since the 3-dimensional structure of PLPs spatially hinders binding to proteolytic enzyme sites. The PLP platform is highly versatile and modular due to synthetic control over the displayed peptides and/or small molecules and the wide range of available peptides and peptide modifications Additionally, peptides, compared to small molecules, provide many more degrees of freedom in respect to binding sites for target engagement, allowing for strong interactions with featureless surfaces.

HYDRACs are heterobifunctional polymeric compounds consisting of at least two distinct sidechain domains. In embodiments, the at least two distinct sidechain domains comprises a protein-targeting ligand and a proteasome recruiting degron. In some embodiments, the at least two distinct sidechain domains comprises a protein-targeting ligand and a nuclear localization sequence. In some embodiments, the at least two distinct sidechain domains comprises a protein-targeting ligand, a proteasome recruiting degron, and a nuclear localization sequence. The chemical nature of their synthesis allows for fine-tuned modulation of both the relative ratios and spatial distribution of each domain component. The HYDRAC platform allows for simultaneous attachment of multiple sequences engaging the same or even different sites of the target protein, providing avidity effects that are not possible with small molecules alone. Multiplexing synergistic degron sequences (i.e. recruit different degradation pathways) provides a level of coverage and reduces chances of resistance arising that cannot be matched by other technologies.

In an aspect, the invention provides a polymer that comprises at least one first repeating unit comprising a first polymer backbone group directly or indirectly covalently linked to a first functional sidechain comprising a Myc binding peptide. In another aspect, the invention provides a polymer that comprises at least one first repeating unit comprising a first polymer backbone group directly or indirectly covalently linked to a first functional sidechain comprising a Myc binding peptide, as well as at least one second repeating unit comprising a second polymer backbone group directly or indirectly covalently linked to a second functional sidechain comprising a degrader agent. In yet another aspect, the invention provides a polymer that comprises at least one first repeating unit comprising a first polymer backbone group directly or indirectly covalently linked to a first functional sidechain comprising a Myc binding peptide, at least one second repeating unit comprising a second polymer backbone group directly or indirectly covalently linked to a second functional sidechain comprising a degrader agent, as well as at least one third repeating unit comprising a third polymer backbone group directly or indirectly covalently linked to a third functional sidechain comprising a nuclear localization peptide.

In aspects, the inventive polymer comprises from 2 to 1000 first repeating units. In other embodiments, the polymer comprises at least 2 first repeating units, and optionally at least 5 first repeating units (e.g., 2-30, 5-30, 10-30, 15-30, or 20-30 first repeating units); wherein each of the first repeating units comprises a first polymer backbone group directly or indirectly covalently linked to a first functional sidechain comprising a Myc binding peptide. Thus, at least one functional sidechain (e.g., the first functional sidechain) of the inventive polymer comprises a Myc binding peptide.

In aspects, the inventive polymer comprises from 0 to 1000 second repeating units. In other embodiments, the polymer comprises at least 1 second repeating unit, optionally at least 2 second repeating units, and optionally at least 5 second repeating units (e.g., 2-30, 5-30, 10-30, 15-30, or 20-30 second repeating units); wherein each of the second repeating units comprises a second polymer backbone group directly or indirectly covalently linked to a second functional sidechain comprising a degrader agent. Thus, in these aspects, at least one functional sidechain (e.g., the second functional sidechain) of the inventive polymer comprises a degrader agent.

In aspects, the inventive polymer comprises from 0 to 1000 third repeating units. In other embodiments, the polymer comprises at least 1 third repeating unit, optionally at least 2 third repeating units, and optionally at least 5 third repeating units (e.g., 2-30, 5-30, 10-30, 15-30, or 20-30 third repeating units); wherein each of the third repeating units comprises a third polymer backbone group directly or indirectly covalently linked to a third functional sidechain comprising a nuclear localization peptide. Thus, in these aspects, at least one functional sidechain (e.g., the third functional sidechain) of the inventive polymer comprises a nuclear localization peptide.

For each of the polymers comprising at least 3 repeating units (e.g., a polymer comprising at least one of the first repeating unit and one or more of: the second repeating unit, the third repeating unit, and a repeating unit having a composition different from the first repeating unit, the second repeating, and the third repeating unit), it will be understood that the repeating units may be arranged in any suitable order. For example, the repeating units can be arranged as a random or statistical polymer, a block polymer, brush, brush block, alternating, segmented, grafted, tapered and other architectures. In embodiments, the repeating units of the polymer are arranged as a random or statistical polymer or as a block polymer. In embodiments, the repeating units of the polymer are arranged as a random or statistical polymer.

In certain embodiments, each of the polymer backbone groups of the inventive polymer (e.g., the first polymer backbone group, the second polymer backbone group, the third polymer backbone group) can independently be any suitable monomer capable of undergoing ring opening metathesis or cross metathesis. For example, each of the polymer backbone groups can independently be a substituted or unsubstituted norbornene, oxanorbornene, olefin, cyclic olefin, cyclooctene, or cyclopentadiene. In some embodiments, each of the first polymer backbone group, the second polymer backbone group, and/or the third polymer backbone group is a polymerized norbornene dicarboxyimide monomer. In preferred embodiments, each polymer backbone group of the polymer is a polymerized norbornene dicarboxyimide monomer. Thus, in certain embodiments for each of the polymers characterized by the formula (FX1), (FX2), and (FX3), each B¹ connected to L¹, P¹ or a combination thereof can independently be characterized by the formula (RU4) or (RU5):

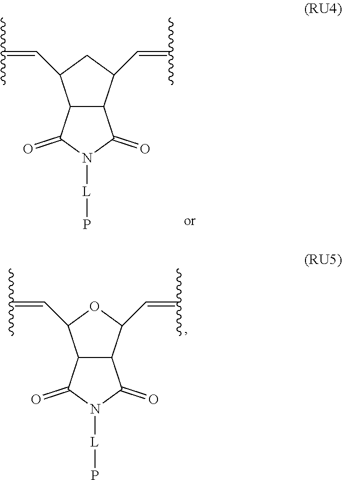

wherein L is L¹ and P is P¹, and when present, each B² connected to L², P² or a combination thereof can independently be characterized by the formula (RU4) or (RU5):

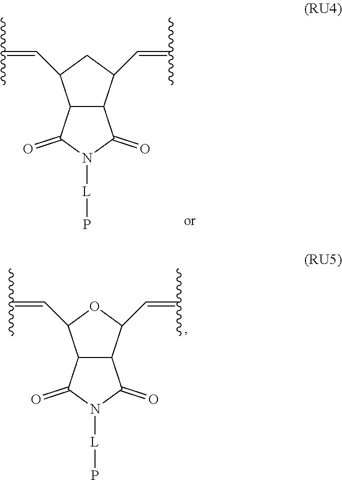

wherein L is L² and P is P², and when present, each B³ connected to L³, P³ or a combination thereof can independently be characterized by the formula (RU4) or (RU5):

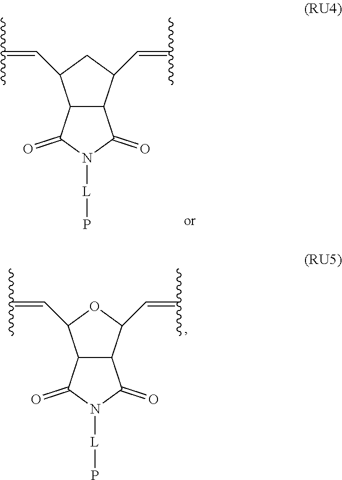

wherein L is L³ and P is P³, and when present, each B⁴ connected to R¹ can independently be characterized by the formula (RU8) or (RU9):

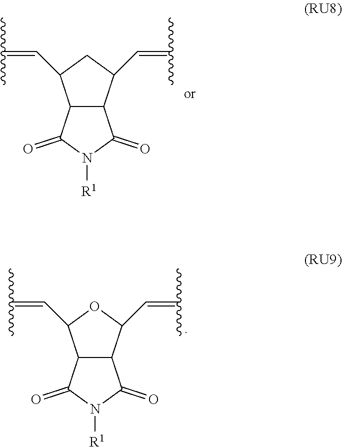

Without subscribing to any particular theory, it is believed that the spacing between the functional sidechains is important for the overall function of the polymers described herein. For example, the relative distances between two individual sidechains falls within a distribution from adjacent to the entire length of the polymer. This is in stark contrast to small molecules alone, which lock the separation of two domains in a rigid manner. Accordingly, in embodiments having a first functional sidechain comprising a Myc binding peptide and one or more functional sidechains independently comprising a degrader agent or a nuclear localization peptide, a single polymer may encompass a range of realized distances between the Myc binding peptide and the degrader agent or the nuclear localization peptide.

In some embodiments, the polymer is characterized by a structure wherein at least a portion of a functional sidechain (e.g., the first functional sidechain, the second functional sidechain, the third functional sidechain) is linked to the polymer backbone group (e.g., the first polymer backbone group, the second polymer backbone group, the third polymer backbone group) via a cleavable linker, such as a matrix metalloproteinase (MMP) cleavage sequence, β-glucuronide linker, cathepsin B cleavage sequence, ester bond, reductive sensitive bond-disulfide bond, hydrazone bond, pH sensitive bond-imine bond or any combinations of these.

In some embodiments, the polymer further comprises a tag for imaging and/or analysis, such as a dye, a radiolabeling agent, an imaging agent, tritiation, and the like. For example, an affinity tag, a solubilization tag, a chromatography tag, an epitope tag, or a fluorescence tag, or any combination thereof. In embodiments, the tag is the result of biotin labeling, fluorescent dye labeling such as cyanine labeling, fluorescein and 7-methoxycoumarin acetic acid labeling, dansyl and/or 2,4-dinitrophenyl labeling, EDANS labeling, coumarin labeling, and/or rhodamine labeling, one or more point mutations, introduction of one or more spacers, isotopic labeling, introduction of one or more chelating agents, acetylation, amidation, methylation, palmitylation, hydroxylation, glycosylation, sulfation and sulfonation, esterification, phosphorylation, peptide stapling, lipidation, cyclization, or any combination thereof. In some embodiments, the polymer comprises a Cy5.5, a biotin tag, a His tag, a FLAG-tag, HA tag, GST tag, or any combination thereof. The tag for imaging and/or analysis may be attached to the polymer in any suitable position. For example, in some embodiments, the tag is directly or indirectly covalently attached to the polymer backbone group. In certain embodiments for each of the polymers characterized by the formula (FXT), (FX2), or (FX3), each T¹, T², L¹, L², and R¹ can independently comprise a tag for imaging and/or analysis. Similarly, each P¹ and P² of formula (FX1), (FX2), or (FX3) (or each P³ and L³ of formula (FX1)) can independently comprise a tag for imaging and/or analysis.

In some embodiments, at least one of the functional sidechains further comprises a charge modulating domain. The charge modulating domain can be any suitable amino acid domain, which increases the positive charge of the peptide. For example, the charge modulating domain can be a TAT sequence, a glycine-serine domain, a cationic residue domain, or a combination thereof. In some embodiments, the charge modulating domain is a glycine-serine domain, a cationic residue domain, or a combination thereof. In certain embodiments, the charge modulating domain is a cationic residue domain having from 2 to 7 amino acid residues selected from lysine, arginine, histidine, and a combination thereof. In preferred embodiments, the charge modulating domain modulates the peptide to have a net positive charge.

After polymerization the inventive polymers may be characterized using any suitable technique(s). Typically, the inventive polymers are characterized by size-exclusion chromatography with multiangle light scattering (SEC-MALS), sometimes referred to as gel permeation chromatography (GPC), to ascertain degree of polymerization (DP) and molecular weight distribution (dispersity or Mw/Mn). Alternatively, or in addition to, the inventive polymers may be characterized by SDS-PAGE to ascertain degree of polymerization (DP) and molecular weight. Preferably, there is suitable agreement between the obtained DP and the theoretical DP based on the initial monomer-to-initiator ratio ([M]₀/[I]₀).

The inventive polymer can have any suitable degree of polymerization. If the degree of polymerization is too low, the polymer may not be resistant to enzymatic cleavage by proteases or may be cleared too rapidly from the body since the polymer's molecular weight would be lower than the clearance threshold through the kidney. Alternatively, if the degree of polymerization is too high, the peptide side chain groups displayed on the polymer may be too dense to engage their biological targets such as PPIs, cell receptors, enzymes, etc. Typically, the polymer has a degree of polymerization of 2 to 1000 (e.g., 2 to 500, 2 to 250, 2 to 100, 2 to 50, 2 to 30, 5 to 1000, 5 to 500, 5 to 250, 5 to 100, 5 to 50, 5 to 30, 20 to 500, 20 to 250, 20 to 100, 20 to 50, or 20 to 30). In certain embodiments, the polymer has a degree of polymerization of 2 to 100. In preferred embodiments, the polymer has a degree of polymerization of 2 to 50. For example, the polymer can have a degree of polymerization of 5 or about 5, a degree of polymerization of 10 or about 10, a degree of polymerization of 15 or about 15 (e.g., 17), a degree of polymerization of 30 or about 30, or a degree of polymerization of 50 or about 50. In certain embodiments, the polymer has a degree of polymerization of at least 5.

The inventive polymer can have any suitable number average molecular weight (Mₙ). The polymers can have a Mₙ of 1,500 kDa or less, for example, 1,000 kDa or less, 800 kDa or less, 600 kDa or less, 400 kDa or less, 200 kDa or less, 100 kDa or less, 90 kDa, or less, 80 kDa, or less, 70 kDa or less, 60 kDa or less, 50 kDa or less, 25 kDa or less, 22 kDa or less, or 20 kDa or less. Alternatively, or in addition, the polymers can have a number average molecular weight of 500 Da or more, for example, 1 kDa or more, 5 kDa or more, 10 kDa or more, 15 kDa or more, 20 kDa or more, or 22 kDa or more. Thus, the polymers can have a number average molecular weight bounded by any two of the aforementioned endpoints.

Generally, the polymers described herein are characterized by a brush density of greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a brush density selected from the range of 60% to 100%, optionally for some embodiments a brush density selected from the range of 70% to 100%, optionally some embodiments a brush density selected from the range of 80% to 100%, or optionally for some embodiments a brush density selected from the range of 90% to 100%.

In another aspect, the invention provides a pharmaceutical composition comprising one or more functional sidechains (e.g., small molecule or peptide) and/or one or more polymers described herein. In some embodiments, the composition comprises one or more pharmaceutically acceptable excipients. For example, the functional sidechains and/or polymers of the invention can be formulated for parenteral administration, such as intravenous (IV) administration or administration into a body cavity or lumen of an organ. Alternatively, the functional sidechains and/or polymers can be injected intra-tumorally. Formulations for injection will commonly comprise a solution of the functional sidechain and/or polymer dissolved in a pharmaceutically acceptable carrier. Among the acceptable vehicles and solvents that can be employed are water and an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the functional sidechain and/or polymer in these formulations can vary widely, and will be selected primarily based on fluid volumes, viscosities, body weight, and the like, in accordance with the particular mode of administration selected and the patient's needs. In certain embodiments, the concentration of a functional sidechain and/or polymer in a solution formulation for injection will range from 0.10% (w/w) to 10% (w/w) or about 0.10% (w/w) to about 10% (w/w).

In some embodiments, the composition further comprises an additional Myc inhibitor or degrader agent. For example the composition can further comprise an additional small molecule drug such as thalidomide or the like. Other small molecule Myc inhibitors or degrader agents will be readily apparent to those skill in the art. In certain embodiments, the composition further comprises an additional Myc inhibiting peptide. In other words, the composition can comprise a PLP described herein and an additional peptide.

In another aspect, the invention provides a method of suppressing transcriptional expression of a target gene in a cell comprising: contacting the cell with an effective amount of a small molecule, peptide, polymer, or pharmaceutical composition described herein.

In another aspect, the invention provides a method of treating or managing a condition comprising administering to a subject an effective amount of a small molecule, peptide, polymer, and/or pharmaceutical composition described herein. The peptide, polymer, and/or pharmaceutical composition can be administered by oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. In some embodiments, the peptide, polymer, and/or pharmaceutical composition is administered intravenously, subcutaneously, intramuscularly, topically, orally, or a combination thereof.

### Aspects of the Invention

Various aspects are contemplated herein, several of which are set forth in the paragraphs below. It is explicitly contemplated that any aspect or portion thereof can be combined to form an aspect. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect, . . . ” means that any aspect prior to aspect B13 is referenced, including aspects B1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y.
Aspect A1: A polymer comprising: a first repeating unit comprising a first polymer backbone group directly or indirectly covalently linked to a first functional sidechain comprising a Myc binding peptide.
Aspect B1: A polymer comprising: a first repeating unit comprising a first polymer backbone group directly or indirectly covalently linked to a first functional sidechain comprising a Myc binding peptide; and
a second repeating unit comprising a second polymer backbone group directly or indirectly covalently linked to a second functional sidechain comprising a degrader agent.
Aspect C1: A polymer comprising: a first repeating unit comprising a first polymer backbone group directly or indirectly covalently linked to a first functional sidechain comprising a Myc binding peptide;
a second repeating unit comprising a second polymer backbone group directly or indirectly covalently linked to a second functional sidechain comprising a degrader agent; and
a third repeating unit comprising a third polymer backbone group directly or indirectly covalently linked to a third functional sidechain comprising a nuclear localization peptide.
Aspect D1: The polymer of any one of aspects A1-C1, wherein the polymer is of formula (FX1):

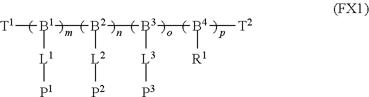

wherein.
T¹ and T² are each independently polymer backbone terminating groups that can be the same or different;
B¹, B², B³, and B⁴ are each independently polymer backbone subunits;
each Li, L², and L³ is optionally present and each is independently a linking group;
each P¹, P², and P³ independently comprises a peptide or a small molecule;
at least one P¹ (e.g., optionally at least half of all P¹, optionally all P¹) independently comprises a sequence having at least 75% or greater (e.g., 75% or greater, 80% or greater, 85% or greater, 90% or greater, 95% or greater, 99% or greater, or 100%) sequence identity of a Myc binding peptide;
at least one P² (e.g., optionally at least half of all P², optionally all P²) independently comprises a sequence having at least 75% or greater (e.g., 75% or greater, 80% or greater, 85% or greater, 90% or greater, 95% or greater, 99% or greater, or 100%) sequence identity of a degrader agent;
at least one P³ (e.g., optionally at least half of all P³, optionally all P³) independently comprises a sequence having at least 75% or greater (e.g., 75% or greater, 80% or greater, 85% or greater, 90% or greater, 95% or greater, 99% or greater, or 100%) sequence identity of a nuclear localization peptide;
each R¹ is independently a substituent;
m is an integer selected from the range of 2 to 1000 (e.g., 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
n is an integer selected from the range of 0 to 1000 (e.g., 0 to 500, 0 to 250, 0 to 100, 0 to 50, 0 to 30, 2 to 1000, 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
o is an integer selected from the range of 0 to 1000 (e.g., 0 to 500, 0 to 250, 0 to 100, 0 to 50, 0 to 30, 2 to 1000, 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
p is an integer selected from the range of 0 to 1000 (e.g., 0 to 500, 0 to 250, 0 to 100, 0 to 50, 0 to 30, 2 to 1000, 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
each connecting line in formula (FX1) represents a covalent linkage comprising at least one of a single bond, a double bond, one or more atoms, or any combination thereof, optionally, for example, each connecting line represents a single bond or double bond;
each instance of B¹, B², B³, B⁴, L¹, L², L¹, R¹, P¹, P², and P³ is the same as or different from any other instance of B¹, B², B³, B⁴, L¹, L², L³, R¹, P¹, P², and P³, respectively; and
the polymer is a block copolymer or a statistical copolymer.
Aspect D2. The polymer of aspect D1, or any preceding aspect, wherein the polymer is of formula (FX2):

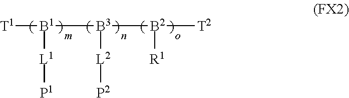

wherein:
T¹ and T² are each independently polymer backbone terminating groups that can be the same or different;
B¹, B², and B⁴ are each independently polymer backbone subunits;
each L¹ and L² is optionally present and each is independently a linking group;
each P¹ and P² independently comprise a peptide or a small molecule;
at least one P¹ (e.g., optionally at least half of all P¹, optionally all P¹) independently comprises a sequence having at least 75% or greater (e.g., 75% or greater, 80% or greater, 85% or greater, 90% or greater, 95% or greater, 99% or greater, or 100%) sequence identity of a Myc binding peptide;
at least one P² (e.g., optionally at least half of all P², optionally all P²) independently comprises a sequence having at least 75% or greater (e.g., 75% or greater, 80% or greater, 85% or greater, 90% or greater, 95% or greater, 99% or greater, or 100%) sequence identity of a degrader agent;
each R¹ is independently a substituent;
m is an integer selected from the range of 2 to 1000 (e.g., 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
n is an integer selected from the range of 0 to 1000 (e.g., 0 to 500, 0 to 250, 0 to 100, 0 to 50, 0 to 30, 2 to 1000, 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
p is an integer selected from the range of 0 to 1000 (e.g., 0 to 500, 0 to 250, 0 to 100, 0 to 50, 0 to 30, 2 to 1000, 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
each connecting line in formula (FX2) represents a covalent linkage comprising at least one of a single bond, a double bond, one or more atoms, or any combination thereof, optionally, for example, each connecting line represents a single bond or double bond;
each instance of B¹, B², B⁴, L¹, L², R¹, P¹, and P² is the same as or different from any other instance of B¹, B², B⁴, L¹, L², RI, P¹, and P², respectively; and
the polymer is a block copolymer or a statistical copolymer.
Aspect D3: The polymer of aspect D1, or any preceding aspect, wherein the polymer is of formula (FX3):

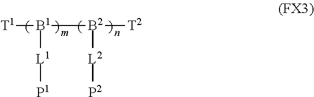

wherein.
T¹ and T² are each independently polymer backbone terminating groups that can be the same or different;
B¹ and B² are each independently polymer backbone subunits;
each L¹ and L² is optionally present and each is independently a linking group;
each P¹ and P² independently comprise a peptide or a small molecule;
at least one P¹ (e.g., optionally at least half of all P¹, optionally all P¹) independently comprises a sequence having at least 75% or greater (e.g., 75% or greater, 80% or greater, 85% or greater, 90% or greater, 95% or greater, 99% or greater, or 100%) sequence identity of a Myc binding peptide;
at least one P² (e.g., optionally at least half of all P², optionally all P²) independently comprises a sequence having at least 75% or greater (e.g., 75% or greater, 80% or greater, 85% or greater, 90% or greater, 95% or greater, 99% or greater, or 100%) sequence identity of a degrader agent;
m is an integer selected from the range of 2 to 1000 (e.g., 2 to 500, 2 to 250, 2 to 100, 2 to 50, 2 to 30, 2 to 20, 5 10 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
n is an integer selected from the range of 0 to 1000 (e.g., 0 to 500, 0 to 250, 0 to 100, 0 to 50, 0 to 30, 2 to 1000, 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 20, 20 to 500, 20 to 250, 20 to 100, 20 to 50, or 20 to 30);
each connecting line in formula (FX3) represents a covalent linkage comprising at least one of a single bond, a double bond, one or more atoms, or any combination thereof, optionally, for example, each connecting line represents a single bond or double bond;
each instance of B¹, B², L¹, L², P¹, and P² is the same as or different from any other instance of B¹, B², L¹, L², P¹, and P², respectively; and the polymer is a block copolymer or a statistical copolymer.
Aspect D4: The polymer of any one of aspects D1-D3, or any preceding aspect, wherein at least one of B¹, B², B³, or B⁴ (e.g., optionally all B¹, B², B³, and B⁴) independently comprises a polymerized monomer comprising an unsaturated monomer.
Aspect D5: The polymer of aspect D4, or any preceding aspect, wherein the unsaturated monomer comprises an ethylenically unsaturated monomer, a norbomene monomer, or a norbornene dicarboxyimide.
Aspect D6: The polymer of any one of aspects D1-D5, or any preceding aspect, wherein each instance of a repeating unit (RU1), (RU2), and (RU3):

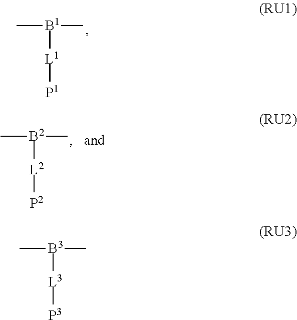

in formula (FX1), (FX2), or (FX3) is independently characterized by a repeating unit (RU4), (RU5). (RU6), or (RU7):

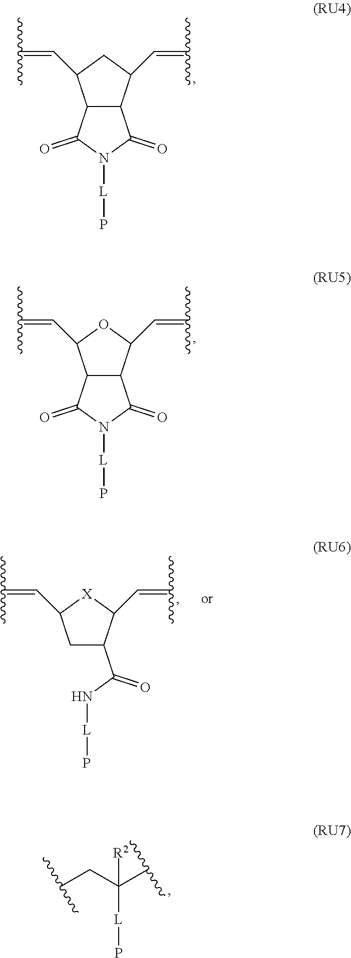

wherein:
L is optionally present and is L¹, L², or L³;
P is P¹, P², or P³;
R² is H or C₁-C₃ alkyl; and
X is CH₂ or O.
Aspect D7: The polymer of aspect D6, or any preceding aspect, wherein each instance of the repeating unit (RU4):

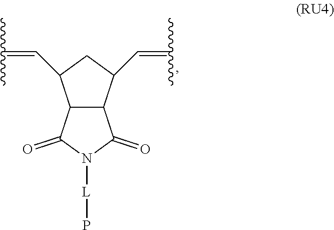

in formula (FX1), (FX2), or (FX3) is independently characterized by a repeating unit (RU4a), (RU4b), or (RU4c):

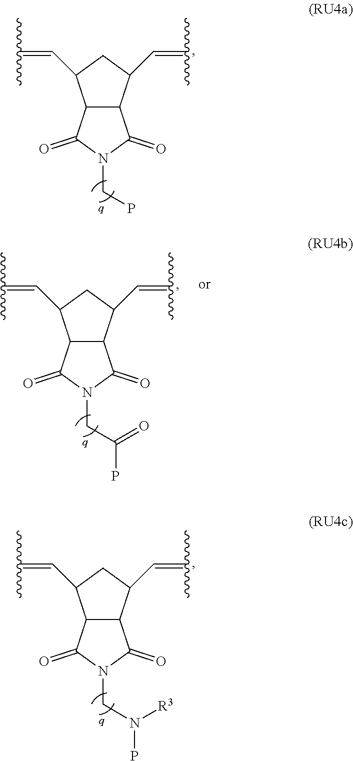

wherein q is an integer from 1 to 20 (e.g., 1, 2, 3, 4, 5, 6, 7, 8, 9, 10, 11, 12, 13, 14, 15, 16, 17, 18, 19, or 20) and R³ is a hydrogen or a C₁-C₅ alkyl.
Aspect D8: The polymer of any one of aspects D1-D7, or any preceding aspect, wherein each of L, L², and L³, is independently selected from a single bond, an oxygen, and groups having an alkylene group, a heteroalkylene group, an alkenylene group, an arylene group, an alkoxy group, an acyl group, a triazole group, a diazole group, a pyrazole group, and combinations thereof.
Aspect D9: The polymer of any one of claims D1-D8, or any preceding aspect, wherein each of L¹, L², and L³, is independently selected from a single bond, —O—, C₁-C₁₀ alkyl, C₂-C₁₀ alkylene, C₁-C₁₀ heteroalkylene, C₃-C₁₀ arylene, C₁-C₁₀ alkoxy, C₁-C₁₀ acyl and combinations thereof.
Aspect D10: The polymer of any one of claims D1-D9, or any preceding aspect, wherein each of L¹, L², and L³, is independently selected from —(CH₂)ₙNR—, —(CH₂)ₙC(O)NR—, —(CH₂)ₙNRC(O)—, —(CH₂)ₙC(O)—and —(CH₂)ₙ—, wherein n is an integer from 1 to 20 (e.g., 1, 2, 3, 4, 5, 6, 7, 8, 9, 10, 11, 12, 13, 14, 15, 16, 17, 18, 19, or 20) and R is hydrogen or a C₁-C₅ alkyl.
Aspect D11: The polymer of any one of claims D1-D10, or any preceding aspect, wherein each of R¹, T¹, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R⁴, —CONR⁵R⁶, —COR⁷, —SOR⁸, —OSR⁹, —SO₂R¹⁰, OR¹¹, SR¹², NR¹³R¹⁴, NR¹⁵COR¹⁶, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R⁴-R¹⁶ independently is H, C₅-C₁₀ aryl, or C₁-C₁₀ alkyl.
Aspect D12: The polymer of any one of claims D1-D11, or any preceding aspect, wherein at least one of R¹, T¹, and T² independently further comprises an analytical tag.
Aspect D13: The polymer of aspect D12, or any preceding aspect, wherein the analytical tag comprises an affinity tag, a solubilization tag, a chromatography tag, an epitope tag, or a fluorescence tag.
Aspect D14: The polymer of any one of aspects D1-D13, or any preceding aspect, wherein at least one of T¹ and T² comprises a degrader agent.
Aspect D15: The polymer of any one of aspects D1-D14, or any preceding aspect, wherein at least one of T¹ and T² comprises a nuclear localization peptide.
Aspect D16: The polymer of any one of aspects D1-D15, or any preceding aspect, wherein at least one of R¹, T¹, and T² independently comprises a cleavable linker.
Aspect D17: The polymer of aspect D16, or any preceding aspect, wherein the cleavable linker is a disulfide linker, a hydrazone, or other suitable acid-labile linker.
Aspect D18: The polymer of aspect D16, or any preceding aspect, wherein the cleavable linker is a β-glucuronide linker, a peptide linker, or other suitable enzymatically cleavable linker.
Aspect D19: The polymer of any one of aspects A1-D18, wherein the polymer is characterized by a number average molecular weight of 1 kDa to 500 kDa (e.g., 1 kDa to 400 kDa, 1 kDa to 250 kDa, 1 kDa to 100 kDa, 1 kDa to 75 kDa, 1 kDa to 50 kDa, 5 kDa to 400 kDa, 5 kDa to 250 kDa, 5 kDa to 100 kDa, 5 kDa to 75 kDa, 5 kDa to 50 kDa, 10 kDa to 400 kDa, 10 kDa to 250 kDa, 10 kDa to 100 kDa, 10 kDa to 75 kDa, 10 kDa to 50 kDa, 20 kDa to 400 kDa, 20 kDa to 250 kDa, 20 kDa to 100 kDa, 20 kDa to 75 kDa, or 20 kDa to 50 kDa).
Aspect D20: The polymer of any one of aspects A1-D18, or any preceding aspect, wherein the polymer is characterized by a number average molecular weight of less than or equal to 50 kDa (less than or equal to 50 kDa, less than or equal to 49 kDa, less than or equal to 48 kDa, less than or equal to 47 kDa, less than or equal to 46 kDa, less than or equal to 45 kDa, less than or equal to 44 kDa, less than or equal to 43 kDa, less than or equal to 42 kDa, less than or equal to 41 kDa, less than or equal to 40 kDa, less than or equal to 39 kDa, less than or equal to 38 kDa, less than or equal to 37 kDa, less than or equal to 36 kDa, less than or equal to 35 kDa, less than or equal to 34 kDa, less than or equal to 33 kDa, less than or equal to 32 kDa, less than or equal to 31 kDa, less than or equal to 30 kDa, less than or equal to 29 kDa, less than or equal to 28 kDa, less than or equal to 27 kDa, less than or equal to 26 kDa, less than or equal to 25 kDa, less than or equal to 24 kDa, less than or equal to 23 kDa, or less than or equal to 22 kDa).
Aspect D21: The polymer of any one of aspects A1-D18, or any preceding aspect, wherein the polymer is characterized by a number average molecular weight of less than or equal to 25 kDa (e.g., less than or equal to 25 kDa, less than or equal to 24 kDa, less than or equal to 23 kDa, less than or equal to 22 kDa, less than or equal to 21 kDa, less than or equal to 20 kDa, less than or equal to 19 kDa, less than or equal to 18 kDa, less than or equal to 17 kDa, less than or equal to 16 kDa, or less than or equal to 15 kDa).
Aspect D22: The polymer of any one of aspects A1-D21, wherein the polymer has an average degree of polymerization from 2 to 1000 (e.g., 2 to 500, 2 to 250, 2 to 100, 2 to 50, 2 to 30, 2 to 20, 5 to 1000, 5 to 500, 5 to 250, 5 to 100, 5 to 50, 5 to 30, 5 to 20, 10 to 250, 10 to 100, 10 to 50, 10 to 30, 10 to 20, 15 to 1000, 15 to 500, 15 to 250, 15 to 100, 15 to 50, 15 to 30, 15 to 25, 20 to 500, 20 to 250, 20 to 100, 20 to 50, or 20 to 30).
Aspect D23: The polymer of any one of aspects A1-D21, or any preceding aspect, wherein the polymer has an average degree of polymerization of 2 to 100 (e.g., 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 15 to 100, 15 to 50, 15 to 30, 15 to 25, 20 to 100, 20 to 50, 20 to 40, or 20 to 30).
Aspect D24: The polymer of any one of aspects A1-D21, or any preceding aspect, wherein the polymer has an average degree of polymerization of 2 to 50 (e.g., 2 to 50, 2 to 40, 2 to 30, 2 to 20, 2 to 10, 4 to 50, 4 to 40, 4 to 30, 4 to 20, 4 to 10, 10 to 50, 10 to 40, 10 to 30, 15 to 50, 15 to 30, 15 to 25, 20 to 50, 20 to 40, or 20 to 30).
Aspect D25: The polymer of any one of aspects A1-D21, or any preceding aspect, wherein the polymer has an average degree of polymerization of 2 to 25 (e.g., 2 to 25, 2 to 20, 2 to 15, 2 to 10, 5 to 25, 5 to 20, 5 to 15, 5 to 10, 8 to 25, 8 to 20, 8 to 15, 10 to 25,or10 to 20).
Aspect D26: The polymer of any one of aspects A1-D25, wherein the polymer has an average length of less than or equal to 100 nm, optionally less than or equal to 80 nm, optionally less than or equal to 60 nm, optionally less than or equal to 50 nm, or optionally less than or equal to 25 nm.
Aspect D27: The polymer of any one of aspects A1-D25, or any preceding aspect, wherein the polymer has an average length of less than or equal to 20 nm, optionally less than or equal to 15 nm, optionally less than or equal to 10 nm, or optionally less than or equal to 5 nm.
Aspect D28: The polymer of any one of aspects A1-D27, wherein the polymer is characterized by an average first repeating unit to second repeating unit ratio of between 1:1 and 15:1 (e.g., between 1:1 and 15:1, between 1:1 and 12:1, between 1:1 and 10:1, between 1:1 and 9:1, between 1:1 and 6:1, or between 1:1 and 5:1).
Aspect D29: The polymer of any one of aspects A1-D27, or any preceding aspect, wherein the polymer is characterized by an average first repeating unit to second repeating unit ratio of between 1:1 and 5:1 (e.g., between 1:1 and 5:1, between 1:1 and 4:1, between 1:1 and 3:1, or between 1:1 and 2:1).
Aspect D30: The polymer of any one of aspects A1-D27, or any preceding aspect, wherein the polymer is characterized by an average first repeating unit to second repeating unit ratio of between 1:1 and 1:15 (e.g., between 1:1 and 1:15, between 1:1 and 1:12, between 1:1 and 1:10, between 1:1 and 1:9, between 1:1 and 1:6, or between 1:1 and 1:5).
Aspects D31: The polymer of any one of aspects A1-D27, or any preceding aspect, wherein the polymer is characterized by proportionate amounts of the first repeating unit, the second repeating unit, and the third repeating unit.
Aspect D32: The polymer of any one of aspects A1-D27, or any preceding aspect, wherein the polymer comprises from 50-75% of the first repeating unit, from 10-30% of the second repeating unit, and from 10-30% of the third repeating unit (for example, 50% of the first repeating unit, 25% of the second repeating unit, and 25% of the third repeating unit; for example, 60% of the first repeating unit, 20% of the second repeating unit, and 20% of the third repeating unit).
Aspect D33: The polymer of any one of aspects A1-D32, wherein the polymer is prepared by a living polymerization method optionally selected from ring-opening metathesis polymerization (ROMP), reversible addition-fragmentation chain transfer polymerization (RAFT), or atom transfer radical polymerization (ATRP).
Aspect D34: The polymer of any one of aspects A1-D33, wherein the polymer is characterized by a high-density brush copolymer having a brush density greater than or equal to 75% (e.g., 75% or greater, 80% or greater, 85% or greater, 90% or greater, 95% or greater, 99% or greater, or 100%).
Aspect D35: The polymer of any one of aspects A1-D33, or any preceding aspect, wherein the polymer is characterized by a high-density brush copolymer having a brush density greater than or equal to 85% (e.g., 85% or greater, 90% or greater, 95% or greater, 99% or greater, or 100%).
Aspect D36: The polymer of any one of aspects A1-D33, or any preceding aspect, wherein the polymer is characterized by a high-density brush copolymer having a brush density greater than or equal to 95% (e.g., 95% or greater, 99% or greater, or 100%).
Aspect D37: The polymer of any one of aspects A1-D33, or any preceding aspect, wherein the polymer is characterized by a high-density brush copolymer having a brush density greater than or equal to 99% (e.g., 99% or greater, or 100%).
Aspect D38: The polymer of any one of aspects A1-D37, wherein at least one functional sidechain comprises a spacer sequence having between 3 and 15 amino acids (e.g., 3, 4, 5, 6, 7, 8, 9, 10, 11, 12, 13, 14, or 15 amino acids).
Aspect D39: The polymer of any one of aspects A1-D38, wherein the first repeating unit is characterized by a number average molecular weight of 1 kDa to 20 kDa (e.g., 1 kDa to 20 kDa, 1 kDa to 15 kDa, 1 kDa to 10 kDa, 1 kDa to 5 kDa, 1.5 kDa to 20 kDa, 1.5 kDa to 15 kDa, 1.5 kDa to 10 kDa, 1.5 kDa to 5 kDa, 2 kDa to 20 kDa, 2 kDa to 15 kDa, 2 kDa to 10 kDa, 2 kDa to 5 kDa).
Aspect D40: The polymer of any one of aspects A1-D38, or any preceding aspect, wherein the first repeating unit is characterized by a number average molecular weight of less than or equal to 10 kDa (e.g., less than or equal to 10 kDa, less than or equal to 7.5 kDa, less than or equal to 5 kDa, or less than or equal to 2.5 kDa).
Aspect D41: The polymer of any one of aspects A1-D38, or any preceding aspect, wherein the first repeating unit is characterized by a number average molecular weight of less than or equal to 5 kDa (e.g., less than or equal to 5 kDa, less than or equal to 4 kDa, less than or equal to 3 kDa, less than or equal to 2.5 kDa, or less than or equal to 2 kDa).
Aspect D42: The polymer of any one of aspects B1-D41, or any preceding aspect, wherein the second repeating unit is characterized by a number average molecular weight of 1 kDa to 10 kDa (e.g., 1 kDa to 10 kDa, 1 kDa to 5 kDa, 1.5 kDa to 10 kDa, 1.5 kDa to 5 kDa, 2 kDa to 10 kDa, 2 kDa to 5 kDa, or 5 kDa to 10 kDa).
Aspect D43: The polymer of any one of aspects B1-D41, or any preceding aspect, wherein the second repeating unit is characterized by a number average molecular weight of less than or equal to 5 kDa (e.g., less than or equal to 5 kDa, less than or equal to 4 kDa, less than or equal to 3 kDa, less than or equal to 2.5 kDa, or less than or equal to 2 kDa).
Aspect D44: The polymer of any one of aspects B1-D41, or any preceding aspect, wherein the second repeating unit is characterized by a number average molecular weight of less than or equal to 2.5 kDa (e.g., less than or equal to 2.5 kDa, less than or equal to 2 kDa, less than or equal to 1.5 kDa, or less than or equal to 1 kDa).
Aspect D45: The polymer of any one of aspects C1-D44, wherein the third repeating unit is characterized by a number average molecular weight of 1 kDa to 20 kDa (e.g., 1 kDa to 20 kDa, 1 kDa to 15 kDa, 1 kDa to 10 kDa, 1 kDa to 5 kDa, 1.5 kDa to 20 kDa, 1.5 kDa to 15 kDa, 1.5 kDa to 10 kDa, 1.5 kDa to 5 kDa, 2 kDa to 20 kDa, 2 kDa to 15 kDa, 2 kDa to 10 kDa, 2 kDa to 5 kDa).
Aspect D46: The polymer of any one of aspects C1-D44, or any preceding aspect, wherein the third repeating unit is characterized by a number average molecular weight of less than or equal to 10 kDa (e.g., less than or equal to 10 kDa, less than or equal to 7.5 kDa, less than or equal to 5 kDa, or less than or equal to 2.5 kDa).
Aspect D47: The polymer of any one of aspects C1-D44, or any preceding aspect, wherein the third repeating unit is characterized by a number average molecular weight of less than or equal to 5 kDa (e.g., less than or equal to 5 kDa, less than or equal to 4 kDa, less than or equal to 3 kDa, less than or equal to 2.5 kDa, or less than or equal to 2 kDa).
Aspect D48: The polymer of aspect A1, or any preceding aspect, further characterized by a number average molecular weight of 1 kDa to 30 kDa (e.g., 1 kDa to 30 kDa, 1 kDa to 20 kDa, 1 kDa to 15 kDa, 1 kDa to 14 kDa, 1.5 kDa to 30 kDa, 1.5 kDa to 20 kDa, 1.5 kDa to 15 kDa, 1.5 kDa to 14 kDa, 2 kDa to 30 kDa, 2 kDa to 20 kDa, 2 kDa to 15 kDa, 2 kDa to 14 kDa, 5 kDa to 30 kDa, 5 kDa to 20 kDa, 5 kDa to 15 kDa, 5 kDa to 14 kDa, 7.5 kDa to 30 kDa, 7.5 kDa to 20 kDa, 7.5 kDa to 15 kDa, or 7.5 kDa to 14 kDa).
Aspect D49: The polymer of aspect A1, or any preceding aspect, further characterized by a number average molecular weight of less than or equal to 20 kDa (e.g., less than or equal to 20 kDa, less than or equal to 15 kDa, less than or equal to 14 kDa, less than or equal to 10 kDa, or less than or equal to 7.5 kDa).
Aspect D50: The polymer of aspect A1, or any preceding aspect, further characterized by a number average molecular weight of less than or equal to 15 kDa (e.g., less than or equal to 15 kDa, less than or equal to 14 kDa, less than or equal to 10 kDa, or less than or equal to 7.5 kDa).
Aspect D51: The polymer of aspect B1, or any preceding aspect, further characterized by a number average molecular weight of 1 kDa to 40 kDa (e.g., 1 kDa to 40 kDa, 1 kDa to 30 kDa, 1 kDa to 20 kDa, 1 kDa to 15 kDa, 5 kDa to 40 kDa, 5 kDa to 30 kDa, 5 kDa to 20 kDa, 5 kDa to 15 kDa, 10 kDa to 40 kDa, 10 kDa to 30 kDa, or 10 kDa to 20 kDa).
Aspect D52: The polymer of aspect B1, or any preceding aspect, further characterized by a number average molecular weight of less than or equal to 30 kDa (e.g., less than or equal to 30 kDa, less than or equal to 20 kDa, less than or equal to 15 kDa, or less than or equal to 12 kDa).
Aspect D53: The polymer of aspect B1, or any preceding aspect, further characterized by a number average molecular weight of less than or equal to 25 kDa (e.g., less than or equal to 25 kDa, less than or equal to 20 kDa, less than or equal to 15 kDa, or less than or equal to 12 kDa).
Aspect D54: The polymer of any one of aspects D1-D53, or any preceding aspect, wherein

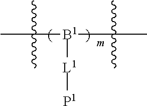

is characterized by a P¹ peptide density of greater than or equal to 80% (e.g., 80% or greater, 85% or greater, 90% or greater, 95% or greater. 99% or greater, or 100%).
Aspect D55: The polymer of any one of claims D1-D53, or any preceding aspect, wherein

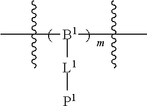

is characterized by a P¹ peptide density of greater than or equal to 90% (e.g., 90% or greater, 95% or greater, 99% or greater, or 100%).
Aspect D56: The polymer of any one of claims D1-D53, or any preceding aspect, wherein

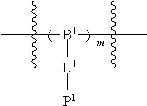

is characterized by a P¹ peptide density of greater than or equal to 95% (e.g., 95% or greater, 99% or greater, or 100%).
Aspect D57: The polymer of any one of aspects D1-D53, or any preceding aspect, wherein

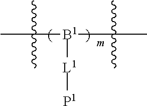

is characterized by a P¹ peptide density of greater than or equal to 99% (e.g., 99% or greater, or 100%).
Aspect D58: The polymer of any one of aspects D1-D57, or any preceding aspect, wherein

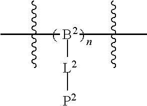

is characterized by a P² peptide density of greater than or equal to 80% (e.g., 80% or greater, 85% or greater, 90% or greater, 95% or greater, 99% or greater, or 100%).
Aspect D59: The polymer of any one of aspects D1-D57, or any preceding aspect, wherein

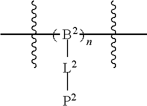

is characterized by a P² peptide density of greater than or equal to 90% (e.g., 90% or greater, 95% or greater, 99% or greater, or 100%).
Aspect D60: The polymer of any one of aspects D1-D57, or any preceding aspect, wherein

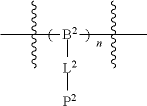

is characterized by a P² peptide density of greater than or equal to 95% (e.g., 95% or greater, 99% or greater, or 100%).
Aspect D61: The polymer of any one of aspects D1-D60, or any preceding aspect, wherein

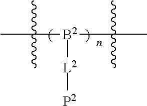

is characterized by a P² peptide density of greater than or equal to 99% (e.g., 99% or greater, or 100%).
Aspect D62: The polymer of any one of aspects D1-D61, or any preceding aspect, wherein

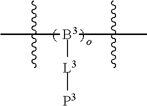

is characterized by a P³ peptide density of greater than or equal to 80% (e.g., 80% or greater, 85% or greater, 90% or greater, 95% or greater, 99% or greater, or 100%).
Aspect D63: The polymer of any one of aspects D1-D61, or any preceding aspect, wherein

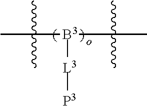

is characterized by a P³ peptide density of greater than or equal to 90% (e.g., 90% or greater, 95% or greater, 99% or greater, or 100%).
Aspect D64: The polymer of any one of aspects D1-D61, or any preceding aspect, wherein

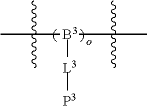

is characterized by a P³ peptide density of greater than or equal to 95% (e.g., 95% or greater, 99% or greater, or 100%).
Aspect D65: The polymer of any one of aspects D1-D61, or any preceding aspect, wherein

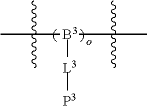

is characterized by a P³ peptide density of greater than or equal to 99% (e.g., 99% or greater, or 100%).
Aspect D66: The polymer of any one of aspects D1-D65, or any preceding aspect, wherein each P¹ independently comprises from 3 to 100 amino acid residues. For example, 3 to 90, 3 to 80, 3 to 70, 3 to 60, 3 to 50, 3 to 40, 3 to 30, 3 to 20, 3 to 16, 3 to 15, 3 to 14, 4 to 90, 4 to 80, 4 to 70, 4 to 60, 4 to 50, 4 to 40, 4 to 30, 4 to 20, 4 to 16, 4 to 15, 4 to 14, 5 to 90, 5 to 80, 5 to 70, 5 to 60, 5 to 50, 5 to 40, 5 to 30, 5 to 20, 5 to 16, 5 to 15, 5 to 14, 6 to 90, 6 to 80, 6 to 70, 6 to 60, 6 to 50, 6 to 40, 6 to 30, 6 to 20, 6 to 16, 6 to 15, 6 to 14, 7 to 90, 7 to 80, 7 to 70, 7 to 60, 7 to 50, 7 to 40, 7 to 30, 7 to 20, 7 to 16, 7 to 15, 7 to 14, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, 9 to 15, 9 to 14, 10 to 16, 10 to 15, 10 to 14, 14 to 20, 14 to 30, 14 to 31, or 14 to 50 amino acid units.
Aspect D67: The polymer of any one of aspects D1-D66, or any preceding aspect, wherein at least one P¹ (optionally at least half of P¹, optionally all P¹) further comprises a therapeutic small molecule.
Aspect D68: The polymer of aspect D67, or any preceding aspect, wherein the therapeutic small molecule is a Myc inhibitor.
Aspect D69: The polymer of any one of aspects D1-D68, or any preceding aspect, wherein at least one P¹ (optionally at least half of P¹, optionally all P¹) comprises an amino acid sequence derived from the amino acid sequence of c-Myc.
Aspect D70: The polymer of any one of aspects D1-D69, or any preceding aspect, wherein at least one P¹ (optionally at least half of P¹, optionally all P¹) comprises an amino acid sequence derived from the amino acid sequence of Max.
Aspect D71: The polymer of any one of aspects D1-D70, or any preceding aspect, wherein at least one P¹ (optionally at least half of P¹, optionally all P¹) comprises a competitive inhibitor peptide.
Aspect D72: The polymer of any one of aspects D1-D71, or any preceding aspect, wherein at least one P¹ (optionally at least half of P¹, optionally all P¹) comprises a c-Myc inhibitor peptide.
Aspect D73: The polymer of any one of aspects D1-D72, or any preceding aspect, wherein at least one P¹ (optionally at least half of P¹, optionally all P¹) is characterized by a helical structure.
Aspect D74: The polymer of any one of aspects D1-D73, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 1 (NELKRAFAALRDQI).
Aspect D75: The polymer of any one of aspects D1-D74, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 2 (NELKRSFFALRDQI).
Aspect D76: The polymer of any one of aspects D1-D75, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 3 (NELKRSFAALRDQI).
Aspect D77: The polymer of any one of aspects D1-D76, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 4 (VQAEEQKLISEEDLLRKRREQLKHKLEQLRN).
Aspect D78: The polymer of any one of aspects D1-D77, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 5 (AEEQKLISEEDLLRKRREQLKHKLEQLRNSC).
Aspect D79: The polymer of any one of aspects D1-D78, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 6 (PGHLKGREIGLWYAKKQGQKNK).
Aspect D80: The polymer of any one of aspects D1-D79, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 7 (HQQDIDDLKRQNALLEQQVRAL).
Aspect D81: The polymer of any one of aspects D1-D80, or any preceding aspect, wherein at least one P¹ (optionally at least half of P¹, optionally all P¹) comprises a sequence having 75% or greater (e.g., 75% or greater, 80% or greater, 85% or greater, 90% or greater, 95% or greater, 99% or greater, or 100%) sequence identity of SEQ ID NO: 8 (HHNALERKRRDHIKDSFHSLRDS).
Aspect D82: The polymer of any one of aspects D1-D81, or any preceding aspect, wherein at least one P² (optionally at least half of P², optionally all P²) comprises a catalyst.
Aspect D83: The polymer of any one of aspects D1-D82, or any preceding aspect, wherein at least one P² (optionally at least half of P², optionally all P²) comprises a proteasome recruiter.
Aspect D84: The polymer of any one of aspects D1-D83, or any preceding aspect, wherein at least one P² (optionally at least half of P², optionally all P²) comprises a peptide capable of binding to an E3 ligase.
Aspect D85: The polymer of any one of aspects D1-D84, or any preceding aspect, wherein each P² independently comprises from 3 to 100 amino acid residues. For example, 3 to 90, 3 to 80, 3 to 70, 3 to 60, 3 to 50, 3 to 40, 3 to 30, 3 to 20, 3 to 16, 3 to 15, 3 to 14, 4 to 90, 4 to 80, 4 to 70, 4 to 60, 4 to 50, 4 to 40, 4 to 30, 4 to 20, 4 to 16, 4 to 15, 4 to 14, 4 to 13, 4 to 12, 4 to 11, 4 to 10, 5 to 90, 5 to 80, 5 to 70, 5 to 60, 5 to 50, 5 to 40, 5 to 30, 5 to 20, 5 to 16, 5 to 15, 5 to 14, 5 to 13, 5 to 12, 5 to 11, 5 to 10, 6 to 90, 6 to 80, 6 to 70, 6 to 60, 6 to 50, 6 to 40, 6 to 30, 6 to 20, 6 to 16, 6 to 15, 6 to 14, 6 to 13, 6 to 12, 6 to 11, 6 to 10, 7 to 90, 7 to 80, 7 to 70, 7 to 60, 7 to 50, 7 to 40, 7 to 30, 7 to 20, 7 to 16, 7 to 15, 7 to 14, 7 to 13, 7 to 12, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, or 9 to 15 amino acid units.
Aspect D86: The polymer of any one of aspects D1-D85, or any preceding aspect, wherein at least one P² (optionally at least half of P², optionally all P²) comprises a small molecule degrader.
Aspect D87: The polymer of aspect D86, or any preceding aspect, wherein the small molecule degrader comprises a thalidomide.
Aspect D88: The polymer of any one of aspects D1-D87, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 9 (RRRG).
Aspect D89: The polymer of any one of aspects D1-D88, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 10 (RRRGN).
Aspect D90: The polymer of any one of aspects D1-D89, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 11 (TRGVEEVAEGVVLLRRRG).
Aspect D91: The polymer of any one of aspects D1-D90, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 12 (TRGVEEVAEGVVLLRRRGN).
Aspect D92: The polymer of any one of aspects D1-D91, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 13 (RQRAIDLFKANELA).
Aspect D93: The polymer of any one of aspects D1-D92, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 14 (ALAPYIP).
Aspect D94: The polymer of any one of aspects D1-D93, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 15 (ALAPYIPR).
Aspect D95: The polymer of any one of aspects D1-D94, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 16 (LDPETGEYL).
Aspect D96: The polymer of any one of aspects D1-D95, or any preceding aspect, wherein at least one P² (optionally at least half of P², optionally all P²) comprises a sequence having 75% or greater (e.g., 75% or greater, 80% or greater, 85% or greater, 90% or greater, 95% or greater, 99% or greater, or 100%) sequence identity of SEQ ID NO: 17 (DRHDSGLDSM).
Aspect D97: The polymer of any one of aspects D1-D96, or any preceding aspect, wherein each P³ independently comprises from 3 to 100 amino acid residues. For example, 3 to90, 3 to 80, 3 to 70, 3 to 60, 3 to 50, 3 to 40, 3 to 30, 3 to 20, 3 to 16, 3 to 15, 3 to 14, 4 to 90, 4 to 80, 4 to 70, 4 to 60, 4 to 50, 4 to 40, 4 to 30, 4 to 20, 4 to 16, 4 to 15, 4 to 14, 4 to 13, 4 to 12, 4 to 11, 4 to 10, 5 to 90, 5 to 80, 5 to 70, 5 to 60, 5 to 50, 5 to 40, 5 to 30, 5 to 20, 5 to 16, 5 to 15, 5 to 14, 5 to 13, 5 to 12, 5 to 11, 5 to 10, 6 to 90, 6 to 80, 6 to 70, 6 to 60, 6 to 50, 6 to 40, 6 to 30, 6 to 20, 6 to 16, 6 to 15, 6 to 14, 6 to 13, 6 to 12, 6 to 11, 6 to 10, 7 to 90, 7 to 80, 7 to 70, 7 to 60, 7 to 50, 7 to 40, 7 to 30, 7 to 20, 7 to 16, 7 to 15, 7 to 14, 7 to 13, 7 to 12, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, or 9 to 15 amino acid units.
Aspect D98: The polymer of any one of aspects D1-D97, or any preceding aspect, wherein at least one P³ (optionally at least half of P³, optionally all P³) is characterized by a net positive charge.
Aspect D99: The polymer of any one of aspects D1-D98, or any preceding aspect, wherein at least one P³ (optionally at least half of P³, optionally all P³) further comprises a charge modulating domain.
Aspect D100: The polymer of aspect D99, or any preceding aspect, wherein the charge modulating domain is a cationic residue domain.
Aspect D101: The polymer of claim D100, or any preceding aspect, wherein the cationic residue domain consists of lysine, arginine, histidine, or a combination thereof.
Aspect D102: The polymer of any one of aspects D1-D101, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 18 (PAAKRVKLD).
Aspect D103: The polymer of any one of aspects D1-D102, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 19 (PKLKRQ).
Aspect D104: The polymer of any one of aspects D1-D103, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 20 (RPRK).
Aspect D105: The polymer of any one of aspects D1-D104, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 21 (RRARRPRG).
Aspect D106: The polymer of any one of aspects D1-D105, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 22 (GKRKLITSEEERSPAKRGRKS).
Aspect D107: The polymer of any one of aspects D1-D106, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 23 (KGKKGRTQKEKKAARARSKGKN).
Aspect D108: The polymer of any one of aspects D1-D107, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 24 (RKRCAAGVGGGPAGCPAPGSTPLKKPRR).
Aspect D109: The polymer of any one of aspects D1-D108, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 25 (RKPVTAQERQREREEKRRRRQERAKEREKRRQERER).
Aspect D110: The polymer of any one of aspects D1-D109, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 26 (RSGGNHRRNGRGGRGGYNRRNNGYHPY).
Aspect D111: The polymer of any one of aspects D1-D110, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 27 (TLLLRETMNNLGVSDHAVLSRKTPQPY).
Aspect D112: The polymer of any one of aspects D1-D111, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 28 (PGKMDKGEHRQERRDRPY).
Aspect D113: The polymer of any one of aspects D1-D112, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 29 (GKKKKGKPGKRREQRKKKRRT).
Aspect D114: The polymer of any one of aspects D1-D113, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 30 (SANKVTKNKSNSSPYLNKRKGKPGPDS).
Aspect D115: The polymer of any one of aspects D1-D114, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 31 (VHSHKKKKIPTSPTFTTPKTLTLRRQPKYPRKSAPRRNKLDHY).
Aspect D116: The polymer of any one of aspects D1-D115, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 32 (RKHKTNRKPR).
Aspect D117: The polymer of any one of aspects D1-D116, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 33 (NRRAKAKR).
Aspect D118: The polymer of any one of aspects D1-D117, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 34 (RNKKKK).
Aspect D119: The polymer of any one of aspects D1-D118, or any preceding aspect, wherein at least one P³ (optionally at least half of P³, optionally all P³) comprises a sequence having 75% or greater (e.g., 75% or greater, 80% or greater, 85% or greater, 90% or greater, 95% or greater, 99% or greater, or 100%) sequence identity of SEQ ID NO: 35 (RKVIK).
Aspect D120: The polymer of any one of aspects D1-D119, or any preceding aspect, wherein at least one P³ (optionally at least half of P³, optionally all P³) comprises a spacer sequence having between 3 and 15 amino acids (e.g., 3, 4, 5, 6, 7, 8, 9, 10, 11, 12, 13, 14, or 15 amino acids).
Aspect E1: A pharmaceutical composition comprising the polymer of any one of aspects A1-D120 and a pharmaceutically acceptable excipient.
Aspect F1: A method of suppressing transcriptional expression of a target gene in a cell comprising:
contacting the cell with an effective amount of the polymer of any one of aspects A1-D120 or the pharmaceutical composition of aspect E1;
wherein the contacting results in the suppressing transcriptional expression of the target gene in the cell.
Aspect F2: The method of aspect F1, wherein the target gene is activated by a Myc/Max heterodimer.
Aspect F3: The method of aspect F1, or any preceding aspect, wherein the contacting results in a disruption of a DNA-protein interaction.
Aspect F4: The method of aspect F1, or any preceding aspect, wherein the contacting results in a disruption of a protein-protein interaction.
Aspect F5: The method of aspect F1, or any preceding aspect, wherein the protein-protein interaction comprises a formation of a Myc/Max dimer.
Aspect F6: The method of aspect F1, or any preceding aspect, wherein the contacting results in a recruitment of a proteasome.
Aspect F7: The method of any one of aspects F1-F6, or any preceding aspect, wherein the cell is a eukaryotic cell.
Aspect F8: The method of aspect F1, or any preceding aspect, wherein transcriptional expression of more than one gene is suppressed.
Aspect F9: The method of any one of aspects F1-F8, or any preceding aspect, wherein the target gene is a pro-proliferative gene.
Aspect G1: A method of targeting a nuclear localized protein in a cell comprising:
introducing the polymer of any one of aspects A1-D120 or the pharmaceutical composition of aspect E1 to a cell;
wherein the introducing results in at least a portion of the polymer or the pharmaceutical composition to bind with at least a portion of the nuclear localized protein;
thereby targeting the nuclear localized protein in a cell.
Aspect G2: The method of aspect G1, wherein the nuclear localized protein is a c-Myc protein.
Aspect G3: The method of aspect G2, or any preceding aspect, wherein the introducing results in at least a portion of the polymer or the pharmaceutical composition to bind with at least a portion of the N-terminal region of the c-Myc protein.
Aspect G4: The method of aspect G2, or any preceding aspect, wherein the introducing results in at least a portion of the polymer or the pharmaceutical composition to bind with at least a portion of the C-terminal region of the c-Myc protein.
Aspect G5: The method of aspect G2, or any preceding aspect, wherein the introducing results in at least a portion of the polymer or the pharmaceutical composition to bind with at least a portion of the bHLHZip domain of the c-Myc protein.
Aspect H1: A method of treating or managing a condition of a subject comprising:
administering to the subject a therapeutically effective amount of the polymer of any one of aspects A1-D120 or the pharmaceutical composition of aspect E1:
wherein the administering results in the treating or managing of the condition of the subject.
Aspect H2: The method of aspect H1 further comprising:
repeating the step of administering to the subject the therapeutically effective amount of the polymer of any one of aspects A1-D120 or the pharmaceutical composition of aspect E1.
Aspect H3: The method of aspect H1 or aspect H2, wherein the method results in an accumulation of proteasomes at the disease site.
Aspect H4: The method of any one of aspects H1-H3, wherein the method results in an accumulation of a Myc binding peptide in a nucleus of a cell.
Aspect H5: The method of any one of aspects H1-H4, wherein the administering to the subject comprises intravenous administration, subcutaneous administration, intramuscular administration, topical administration, oral administration, or a combination thereof
Aspect H6: The method of any one of aspects H1-H5, wherein the condition is a Myc-dependent cancer.
Aspect H7: The method of aspect H6, or any preceding aspect, wherein the method interrupts the protein-protein interaction between c-Myc and Max.
Aspect H8: The method of aspect H6, or any preceding aspect, wherein the method interrupts the DNA-protein interaction between DNA and c-Myc.
Aspect H9: The method of aspect H6, or any preceding aspect, wherein the method inhibits transcriptional activity of c-Myc.
Aspect H10: The method of any one of aspects H1-H3, or any preceding aspect, wherein the condition is a respiratory disease.
Aspect H11: The method of aspect H10, or any preceding aspect, wherein the respiratory disease is COPD, asthma, emphysema, potential treatment for smokers, idiopathic pulmonary fibrosis, chronic sarcoidosis, or hypersensitivity pneumonitis.
Aspect H12: The method of any one of aspects H1-H3, or any preceding aspect, wherein the condition is associated with an inflammatory state, increased oxidative stress, autoimmune pathophysiology, chemo-preventative measures, neurodegeneration, or a combination thereof.
Aspect H13: The method of any one of aspects H1-H3, or any preceding aspect, wherein the condition is a gastrointestinal disease.
Aspect H14: The method of aspect H13, or any preceding aspect, wherein the gastrointestinal disease is ulcerative colitis, ulcers, prevent acetaminophen toxicity, non-alcoholic steatohepatitis, primary biliary cholangitis, cirrhosis, type 2 diabetes, diabetic nephropathy, or Crohn's disease.
Aspect H15: The method of any one of aspects H1-H3, or any preceding aspect, wherein the condition is an autoimmune disease.
Aspect H16: The method of H15, or any preceding aspect, wherein the autoimmune disease is multiple sclerosis, systemic lupus erythematous, Sjogren syndrome, rheumatoid arthritis, vitiligo, or psoriasis.
Aspect H17: The method of any one of aspects H1-H3, or any preceding aspect, wherein the condition is a genetic disease.
Aspect H18: The method of aspect H17, or any preceding aspect, wherein the genetic disease is polycystic kidney disease.
Aspect H19: The method of any one of aspects H1-H3, or any preceding aspect, wherein the condition is an age-associated pathology.

### EXAMPLES

The invention can be further understood by the following non-limiting examples. The examples are provided to illustrate some of the concepts described within this disclosure. While each example is considered to provide specific individual embodiments of composition and methods of preparation and use, none of the examples should be considered to limit the more general embodiments described herein.

In the following examples, efforts have been made to ensure accuracy with respect to numbers used (e.g., amounts, temperature, etc.) but some experimental error and deviation should be accounted for.

In the following examples, unless otherwise stated, “H1” is the peptide-based Myc inhibitor, SEQ ID NO: 1, “degron,” “proteasome recruiter,” or “R” is the degrader agent sequence, SEQ ID NO: 9, “M1” is the nuclear localization sequence, SEQ ID NO: 18, “s” denotes that the compound to which it precedes comprises a randomly scrambled sequence of said compound, and “HYDRAC” refers generally to PLPs which contain heterologous side chains with distinct functionalities, wherein one domain binds to a protein of interest and a second targets it for degradation. Additionally, numerical subscripts associated with any of these terms denote the average DP associated with the compound. In some cases, a range is provided (e.g., H147) indicating the average DP of the compound falls within the indicated range of DP.

Materials and Methods: The following descriptions of materials and methods apply to one or more of the below Examples. To the extent of any conflict between the materials and methods of these descriptions and those provided in the Examples, the Examples control.

Peptide Synthesis: Peptides may be synthesized on a Liberty Blue (CEM) Automated Microwave Peptide Synthesizer using standard solid-support synthesis protocols and Fmoc-protected amino acids. Rink amide MBHA resin was used to give C-terminal amide. The peptides were capped with Norbomene-amino hexanoic acid as previously described in Blum et al., Peptides Displayed as High Density Brush Polymers Resist Proteolysis and Retain Bioactivity, J. Am. Chem. Soc. 2014, 136 (43), 15422-15437, which is hereby incorporated by reference. Cleavage from the resin was performed in 95:2.5:2.5 (% v/v) trifluoroacetic acid (TFA), triisopropyl silane, and water, respectively, for 2 h. Following evaporation of TFA, the cleaved peptides were precipitated in cold diethyl ether and dried under vacuum to yield solid crude.

Peptides were purified with a Jupiter Proteo 90 Å Phenomenex column (2050×25.0 mm) on an Armen Glider CPC preparatory phase HPLC to yield 90-95% purity, confirmed by analytical HPLC. For all RP-HPLC purifications, gradient solvent systems utilized Buffer A (water with 0.1% TFA) and Buffer B (acetonitrile with 0.1% TFA). A gradient of 15-45% buffer B over 1 h was used to purify all peptides and monomers. Pure products were then analyzed by electrospray ionization mass spectroscopy (ESI-MS) on a Bruker amaZon SL to confirm molecular weight.

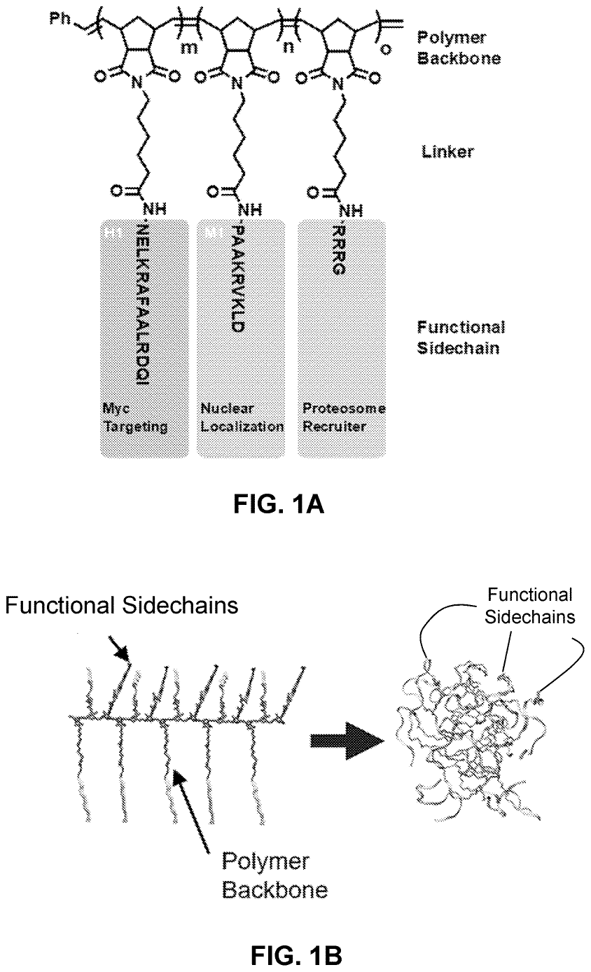

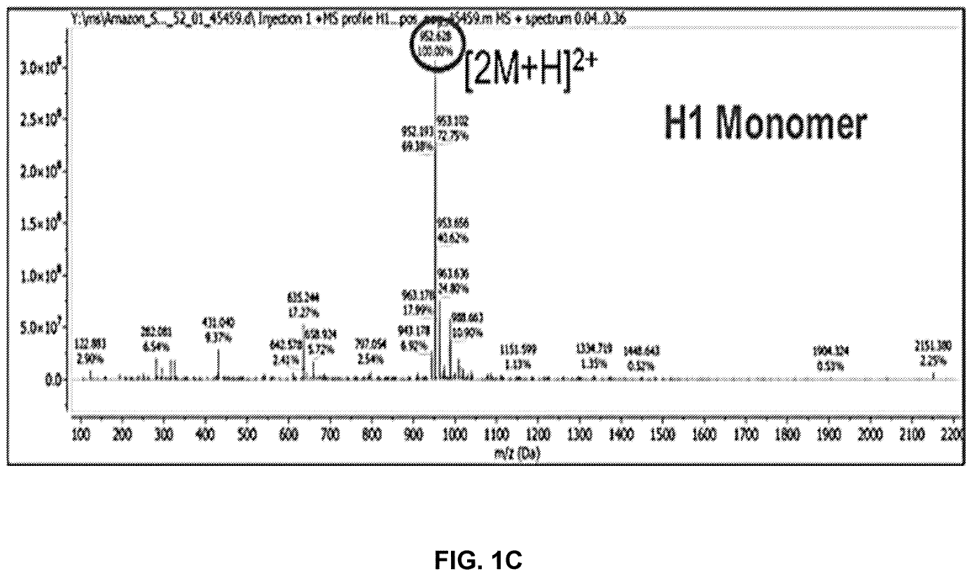

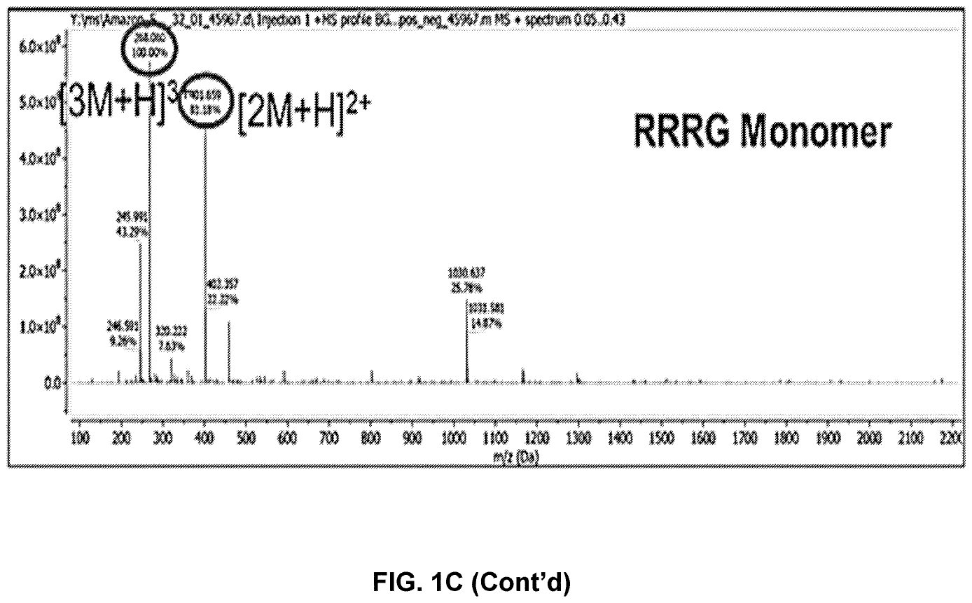

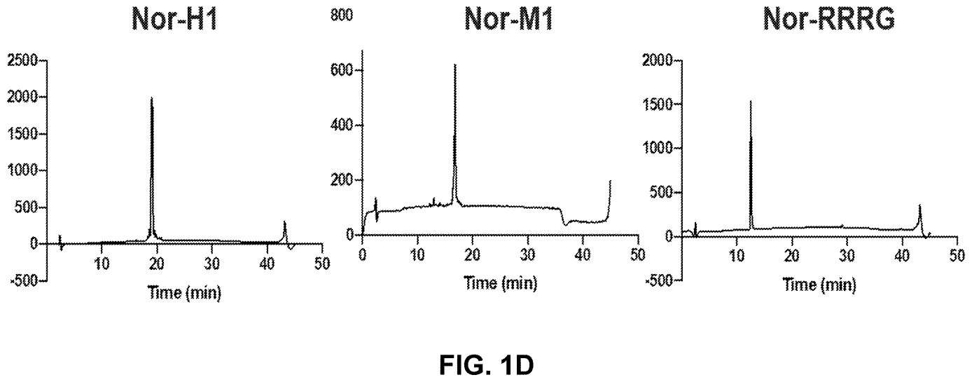

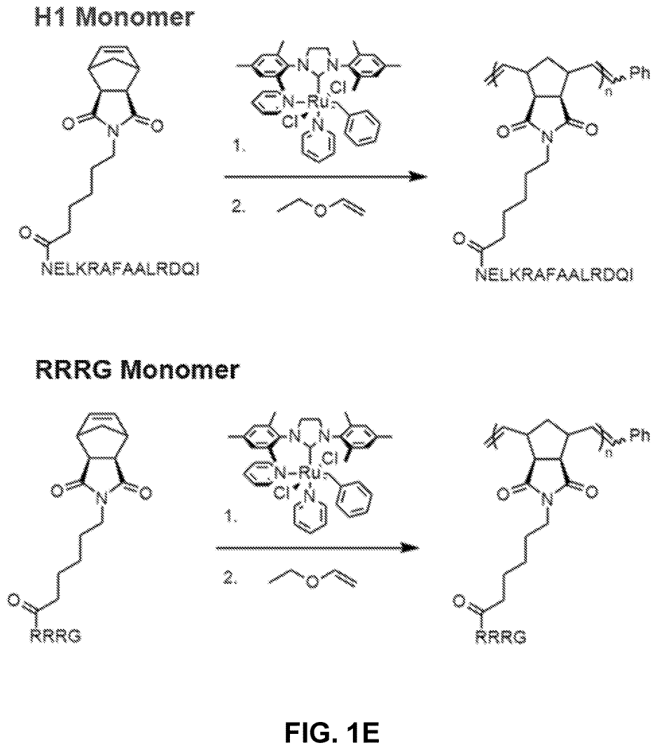

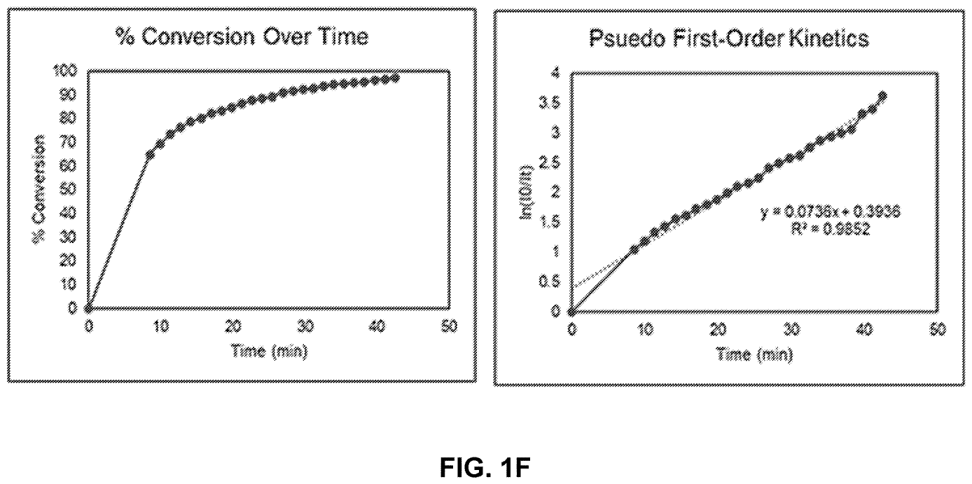

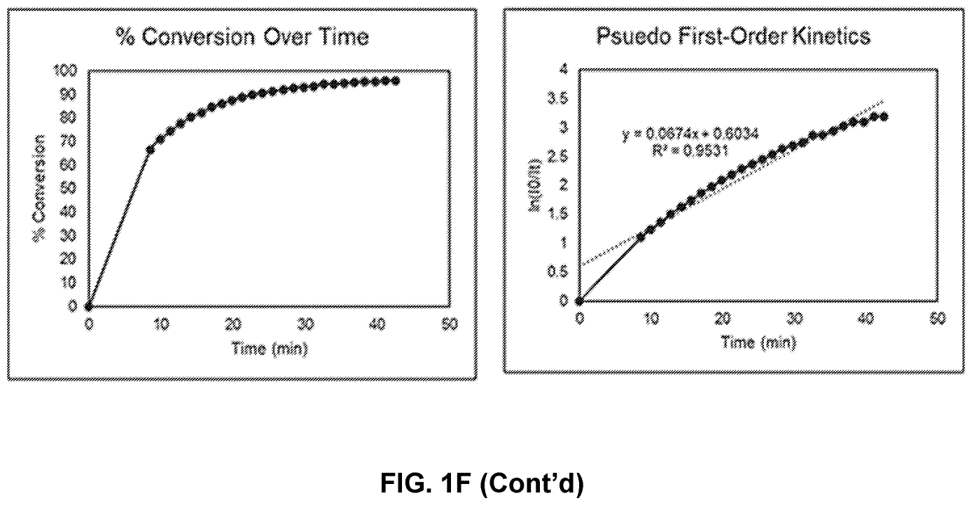

Polymerization: Protein-Like Polymers (“PLPs”) incorporating the purified peptides were synthesized following the general mechanism provided in FIG. 1E, aspects of which are further described in Kammeyer et al., Polymerization of Protecting-Group-Free Peptides via ROMP, Polym. Chem. 2013, 4 (14), 3929-3933 and Nomura et al., Precise Synthesis of Polymers Containing Functional End Groups byLiving Ring-Opening Metathesis Polymerization (ROMP): Efficient Tools for Synthesis ofBlock/Graft Copolymers, Polym. 2010, 51(9), 1861-1881, which are hereby incorporated by reference. Polymerization reactions were carried out in a glovebox under a nitrogen atmosphere as described in Blum et al. A typical protocol used to generate a polymer with DP=15 involved mixing the monomer (e.g., 15 mg, 0.0103 mmol) with the initiator (e.g., 0.5 mg, 0.00068 mmol) in dry DMF (0.5 mL). 1H NMR was used to confirm complete consumption of the monomer and to determine the time period required to reach completion. The percent conversion of monomer to polymer was tracked over time by comparing a decreasing monomer peak by NMR relative to an internal standard. The linear plot of the NMR revealed the pseudo-first-order kinetics. The polymers were terminated with ethyl vinyl ether (10 eq) for I h at room temperature, precipitated and washed with cold diethyl ether three times and collected by centrifugation. Polymers molecular weight and polydispersity were determined by SEC MALS (Phenomenex Phenogel 5p 103A, 1K-75K, 300×7.8 nmmi in series with a Phenomenex Phenogel 5p 103A, 10K-100K, 300×7.8 mm) at 65° C. in 0.05M LiBr in DMF, using a ChromTech Series 1500 pump equipped with a multi-angle light scattering detector (DAWN-HELIOS II, Wyatt Technology) and a refractive index detector (Wyatt Optilab TrEX) normalized to a 30,000 MW polystyrene standard.

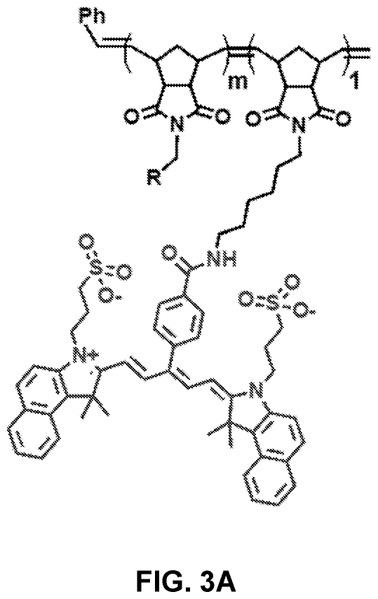

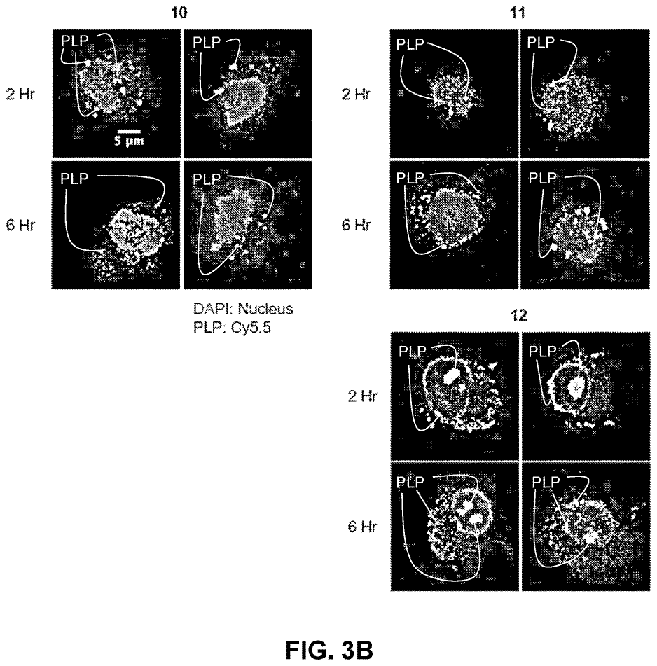

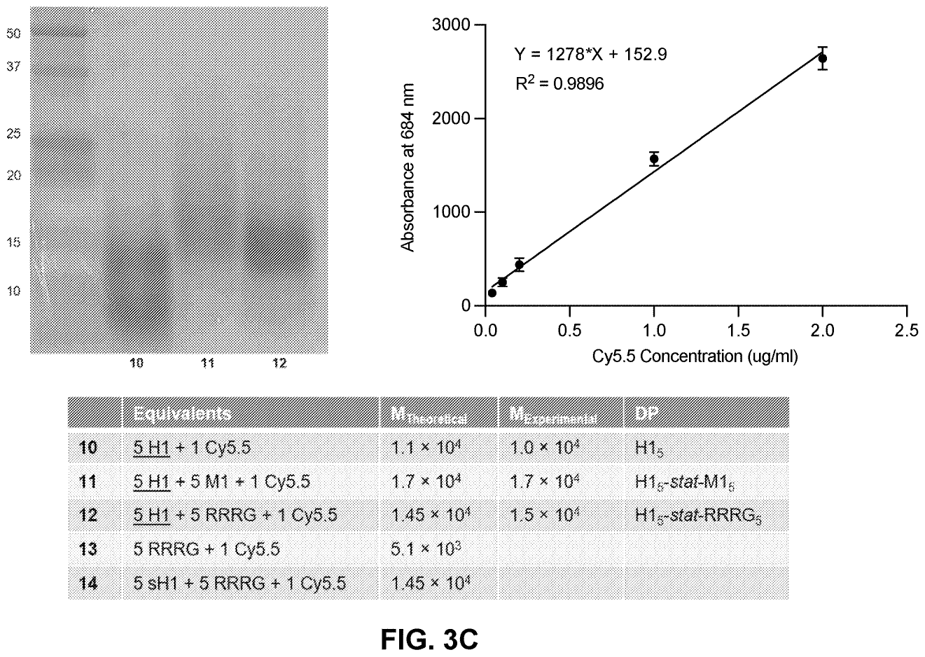

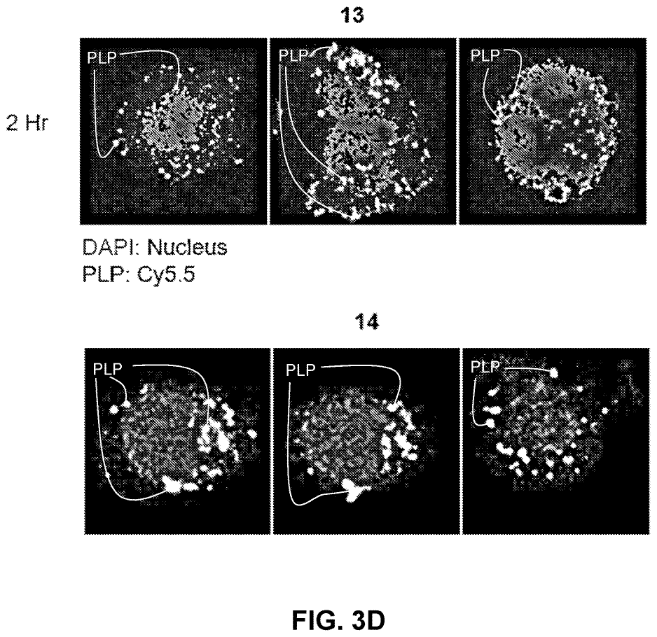

Depending on the application of the inventive polymers, a final termination step can be modified by terminating with a dye or another small molecule, providing further functionalization. For example, the use of Cy5.5 (e.g., FIG. 3A) as well as biotin-labeled polymers obtained by synthetically manipulating the termination step may be used. PLPs may be prepared from peptide monomers, used without the need for special protecting groups or non-natural amino acids, aside from the case of cysteine residues which should be protected during polymerization. No special design requirements are enforced on the sequences used. It is believed that the degree of polymerization is controlled by modulating the monomer to initiator ratio, resulting in a controlled polymer architecture with low dispersity. Furthermore, where PLPs are prepared using ROMP, a living polymerization method, regulating the addition of peptide monomers to the initiator gives control over the final polymer composition (block, random etc.).

For example, to generate a block copolymer, NMR kinetics were used to first track the rate of each monomer's conversion to polymer (e.g., FIG. 1F & FIG. 1F (Cont'd.)), so when the conversion of one monomer had been carried out, an additional monomer could be added after. Conversely, two different monomers were added at the same time to create random (statistical) copolymers. In some cases, monomers having similar rates of conversion resulted in preferential statistical copolymers.

Circular Dichroism (CD) Spectroscopy: CD spectra were obtained on a JASCO J-815 spectrometer equipped with a Peltier sample holder for temperature control. Polymer samples were dissolved in buffered solvent to concentrations of approximately 1 μM, and placed into a 1 cm quartz cuvette (Hellma). Protein samples were measured at 5 μM. All samples were equilibrated at room temperature for at least thirty minutes prior to measurement, with mixtures (i.e. protein and polymer combinations) being generally equilibrated for 12 hours prior to measurement. Spectra were obtained using a scan speed of 10 nm/min, using a 1 nm bandwidth, 4 sec response time, and with three accumulations. Background spectra of solvent were obtained prior to measurement of the sample solutions, and subtracted from the sample spectra using JASCO Spectra Analysis. For melting experiments, the temperature was varied between 2° and 91° C. with steps of 1° C./min, while measuring the ellipticity at 222 nm with a 2 nm bandwidth.

In vivo Studies: All animal experiments and procedures were performed in compliance with ethical standards and the approval of Northwestern University Animal Care and Use Committee (IACUC). FVB mice were obtained from Jackson Laboratory. All the mice used in this study were maintained in a pathogen-free animal barrier facility. All the experiments were initiated with mice of age 6 to 8 weeks. For allograft models, MYC-driven murine prostate cancer Myc-CaP cells were suspended at a concentration of 10 million cells/mL in 50% Matrigel-50% PBS and 100 μL of this solution for a total of 1 million cells subcutaneously injected into the flanks of mice. Mice were grouped and treatment was started when the tumor size reached around 150 to 180 mm³. PLP was dissolved in 10% DMSO (Sigma) in PBS for I.P. administration.

Cell Culture: PC-3, PC-12, A549 cells were obtained from ATCC; Mouse MycCaP cells were the kind gift of Charles Sawyers (Memorial Sloan-Kettering Cancer Centre). All cells were authenticated and tested as mycoplasma-free. PC-3 and A549 cells were grown in F12K (Gibco) medium; MycCaP cells in DMEM medium (Gibco), all supplemented with 10% heat-inactivated fetal bovine serum (FBS, Gibco). PC-12 cells were grown in F-12K medium (ATCC) with 5% heat-inactivated FBS and 10% heat-inactivated horse serum (Thermo). All cells were cultured in 1% Penicillin-Streptomycin (10,000U/ml, Life Technologies) and 5% CO₂ in a humidified incubator at 37° C.

Cell Proliferation Assays: Cell viability was estimated using the CellTiter-Glo® Luminescent Cell Viability Assay (Promega). According to cell type and experimental setting, 1000 to 5000 cells/well were seeded in 96 well white plates with clear bottom (ThermoFisher, 136101) and allowed to adhere overnight. After 3 to 5 days following the treatment, CellTiter-Glo reagent was added mixed 1:1 vol/vol with fresh media, plates incubated for 10 minutes on a shaking rotator followed by 10 minutes without shaking at room temperature (protected from light using aluminum foil) and luminescence was measured using plate reader (Perkin Elmer Victor 3V).

Western BlotAnalysis: Cells were lysed in RIPA (Sigma) lysis buffer containing 1×Halt™ Protease and Phosphatase Inhibitor Cocktail (ThermoFisher). Protein concentration was measured by Bio-Rad Bradford reagent. Protein samples were prepared by addition of 4×Laemmli Sample buffer (Bio-Rad) and 2-mercaptoethanol (Bio-Rad) and resolved on 4-12% SDS-PAGE (Sodium dodecyl sulfate-polyacrylamide) gels, which were subsequently transferred to PVDF membranes (Bio-Rad) using Trans-Blot Turbo transfer buffer (Bio-Rad) and a Trans-Blot Turbo Transfer System (Bio-Rad). Membranes were blocked for 1 h at room temperature with 5% blotting-grade blocker non-fat dry milk (Bio-Rad), followed by overnight 4° C. incubation with the appropriate primary antibody and 1 h room temperature incubation with an anti-rabbit or anti-mouse IgG (H+L)-HRP conjugate (Bio-Rad) secondary antibody. Blots were imaged using Supersignal West Femto Maximum Sensitivity Substrate detection system (Thermo) and the ChemiDoc Imaging System (Bio-Rad). The following primary antibodies were used: c-Myc (Y69) (Abcam #ab32072), Streptavidin-HRP (PerkinElmer NEL750), GAPDH (Cell Signaling #3683) and Actin (Cell Signaling #5125). Quantification analyses were performed by Biorad ChemiDoc Imager and Bio-Rad Image Lab software.

In vitro Pull Down Assay: To confirm PLP direct binding to endogenous Myc protein in cell lysate complex, biotin conjugated PLP was synthesized. Collected PC-3 cell pellet (2 million cells per pulldown condition) was suspended in 300 μL pulldown lysis buffer (pH 7.4 50 mM Tris-HCl, 150 mM NaCl, 1 mM EDTA, 0.1% IGEPAL nonionic detergent+1× Halt protease/phosphatase inhibitor cocktail) and lysed by repeat (3×) freeze-thawing in liquid nitrogen. The lysed cell pellets were spun at 4° C. for 15 minutes at 15,000 rpm on a Eppendorf 5424R centrifuge. The collected supernatants were pre-cleared with Pierce streptavidin magnetic beads (Thermo, 88817) for 1 hr at 4° C. For Examples 7 and 10, around 300 μg was applied to each sample and incubated with 5 μM of HYDRAC, scrambled HYDRAC, 10 μM of H1 homopolymer, scrambled H1 homopolymer or D-Biotin on a rotator over night at 4° C. Next day, 60 μl of streptavidin beads was added to each sample and further rotated for 1 hr at 4° C. Beads were washed with wash buffer (lysis buffer containing 0.1% BSA) for 3 times and another 3 times with lysis buffer, then eluted with 50 μL of 2×Laemmli sample buffer and boiled at 95° C. for 5 min. The supernatant was subjected to Western Blot.

RNA-Sequencing: PC-3 cells were treated with 5 μM PLP or 5 μM scramble control. Cells were collected using trypsinization and washed with ice-cold PBS and later centrifuged at 1500 rpm for 5 minutes. Total RNA was extracted from cell pellet using Qiagen RNeasy Plus kit. The stranded total RNA-seq was conducted by the Northwestern University NUSeq Core Facility. Briefly, total RNA examples were checked for quality on Agilent Bioanalyzer 2100 and quantity with Qubit fluorometer. The NEBNext Ultra II RNA Library Prep Kit for Illumina was used to prepare sequencing libraries. The Kit procedure was performed without modifications. This procedure includes rRNA depletion, remaining RNA purification and fragmentation, cDNA synthesis, end preparation, Illumina adapter ligation, library PCR amplification and validation. IlluminaHiSeq 4000 Sequencer was used to sequence the libraries with the production of single-end, 50 bp reads.

Mass Spectrometry-Based Whole Proteome Analysis: PC-3 cells were treated with indicated polymer compositions for 24 hours. After treatment, the cells were collected and lysed using a probe sonicator (40%, 3, 5 pulses) in 150 μL of lysis buffer (PBS supplemented with Roche complete protease inhibitor cocktail). The protein concentration in the lysate was determined using a DC assay and adjusted to 1.0 mg/mL. Next, 100 μL of protein samples containing 100 μg of protein were transferred to new Eppendorf tubes (1.5 mL), to which 48 mg of urea was added (final concentration is 8 M). Subsequently, 5 μL of DTT (200 mM stock in water) was added to the tubes to achieve a final concentration of 10 mM. The samples were then incubated at 65° C. for 15 min. Afterward, 5 μL of iodoacetamide (400 mM stock in water) was added to the tubes to achieve a final concentration of 20 mM. The samples were then incubated in the dark at 37° C. for 30 min. Next, 600 μL of MeOH, 200 μL of CHCl3, and 500 μL of water were added to the tubes, mixed, and then centrifuged at 10,000 g for 10 min at 4° C. A protein disc was formed at the interface of the CHCl3 and aqueous layers. The top layer was aspirated, and 1 mL of MeOH was added. The samples were then pelleted after centrifugation (10,000 g, 10 min, 4° C.).

The protein pellets were resuspended in 160 μL of EPPS buffer (200 mM, pH 8). Next, 4 μL of LysC solution (0.5 μg/μL in water) was added to each sample, and the samples were incubated at 37° C. for 2 h. Afterward, 10 μL of trypsin (0.5 μg/μL in trypsin buffer) and 1.8 μL of CaCl₂) (100 mM stock in water) were added to each sample, and the samples were incubated at 37° C. for 12 h. The peptide concentration was determined using a micro BCA assay kit. For each sample, a volume corresponding to 12.5 μg of peptides was transferred to a new Eppendorf tube, and the total volume was adjusted to 35 μL using EPPS buffer (200 mM, pH 8). Next, 9 μL of CH₃CN was added to each sample, followed by the addition of 6 μL of TMT tags (20 μg/μL in dry CH₃CN). The samples were then incubated at room temperature for 1 hour. To quench the TMT labeling reaction, 6 μL of hydroxylamine (5% in water) was added to each sample. After incubating at room temperature for 15 minutes, 2.5 μL of formic acid was added. The samples were combined into a new Eppendorf tube and dried using a SpeedVac. Subsequently, the peptides were fractionated into 12 fractions using the Thermo Vanquish UHPLC fractionator.

The peptides were analyzed using liquid chromatography tandem mass-spectrometry on an Orbitrap Eclipse Tribrid Mass Spectrometer coupled to a Vanquish Neo UHPLC System. Peptides were injected onto an EASY-Spray HPLC column (C18, 2 μm particle size, 75 μm inner diameter, 250 mm length) and separated at a flow rate of 0.25 μL/min using the following gradient: 5% buffer B (80% acetonitrile with 0.1% formic acid) in buffer A (water with 0.1% formic acid) from 0-15 min, 5-45% buffer B from 15-155 min, and 45-100% buffer B from 155-180 min. The nano-LC electrospray ionization source was set to a voltage of 1.5 kV. The scan sequence began with an MS1 master scan (Orbitrap analysis, resolution 120,000, 375-1600 m/z, RF lens 30%, standard AGC target, auto maximum injection time). The top ten precursors were then selected for MS2/MS3 analysis. MS2 analysis involved quadrupole isolation (isolation window 1.2) of precursor ion, followed by HCD collision entry in the ion trap (standard AGC, collision energy 32%, maximum injection time 35 ms). Following the acquisition of each MS2 spectrum, synchronous precursor selection (SPS) enabled the selection of up to 10 MS2 fragment ions for MS3 analysis. The MS3 precursors were fragmented by HCD and analyzed using the Orbitrap (collision energy 55%, AGC 250%, maximum injection time 200 ms, resolution was 50,000). The RAW data was searched in Proteome Discoverer 2.5.

Immunofluorescence andImmunohistochemistry: Tissues were fixed in 10% neutral buffered formalin for 48 hr at 4° C. and transferred to 70% ethanol before paraffin processing at the Northwestern University Mouse Histology and Phenotyping Laboratory (MHPL) histology core. Primary antibodies used are: c-Myc (Y69) (Abcam #ab32072), Ki-67 (Abcam #ab15580), Cleaved Caspase-3 (Cell Signaling #9661). For Immuno-histochemistry, slides were incubated with ImmPRESS HRP anti-mouse (Vector #MP-7402) or ImmPRESS HRP anti-rabbit (Vector #MP-7401). Expression was visualized by using AEC peroxidase substrate (Vector #SK-4200). Slides were incubated with Hematoxylin (Vector #3404) and mounted with Glycergel Mounting Medium (Dako #C056330-2). For immuno-fluorescence, slides were incubated with secondary antibodies labelled with Alexa Fluor 488 anti-rabbit (Thermo Scientific #A11008), Alexa 594 anti-rabbit (Thermo Scientific #A-21207), Alexa 594 anti-mouse (Molecular probes #A11005) and Alexa Fluor 568 Goat anti-Mouse IgG (H+L) (Thermo Scientific #A-11004). Slides were counterstained with DAPI (Sigma #D-9542) and mounted with ProLong Gold Antifade reagent (Invitrogen/Molecular Probes #P36961). Immunofluorescence images were visualized using fluorescent microscope or Leica AIR spectral confocal microscope.

Hemolysis Assay: RBCs were isolated by centrifugation of 40 mL citrated whole human blood at 500×g for 5 min, followed by gentle washes with 150 mM NaCl (×1) and 1×DPBS (×3). Isolated RBCs were diluted 1:50 into 1×DPBS (pH 7.4) and lightly agitated to prevent settling. Briefly, 190 μL of dilute RBCs and 10 μL of polymer stock or additive (20× final concentration) were mixed and added to a 96-well plate (n=4 repeats), covered, and incubated at 37° C. for 1 h. Plates were centrifuged at 500×g to pellet intact RBCs using a centrifuge equipped with a microplate rotor. Supernatant (100 PL) was transferred to a new 96-well plate, taking care not to disturb the pellet. Absorbance of the supernatant was measured at 540 nm to detect released hemoglobin. To calculate % hemolysis, absorbances were corrected for background absorbance from untreated vehicle and then normalized to 1% Triton X-100-treated RBCs to represent 100% hemolysis.

ACT with Whole Human Blood: A Hemochron 801 instrument calibrated with an electronic system verification (ESV) device was used to measure ACT of whole human blood. ACTs were determined using recalcified citrated whole human blood to minimize variability in starting time points for clotting in all assays. To each Hemochron P214 tube with glass beads was added 4 μL CaCl₂) (1.1 M) and 36 μL polymer stock or additive (12.2× final blood concentration). Samples were mixed thoroughly for 30 s to soak the glass beads and incubated for 30 s at 37° C. Citrated whole human blood (400 μL) was then added (t=0 s), mixed by hand for 10 s, and added to the instrument. Time points at which the magnet was displaced by clotformation were recorded by the instrument. Collagen (0.095 mg mL-1) was used as a positive control to decrease clotting time. Vehicle (1×DPBS) served as a standard for blood without additive. Samples without calcium, serving as the negative control, exceeded instrument maximum time range (>1500 s).

### Example 1

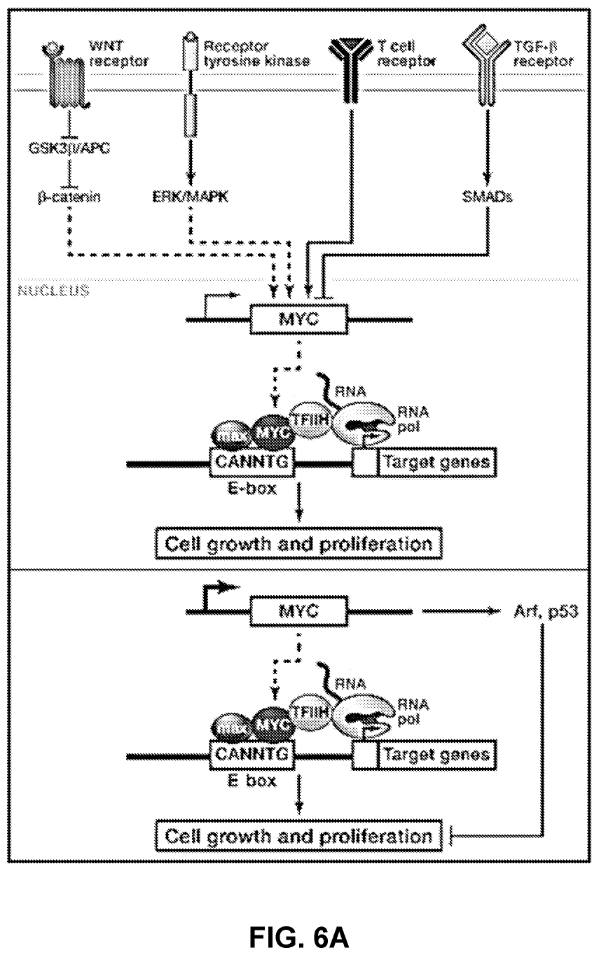

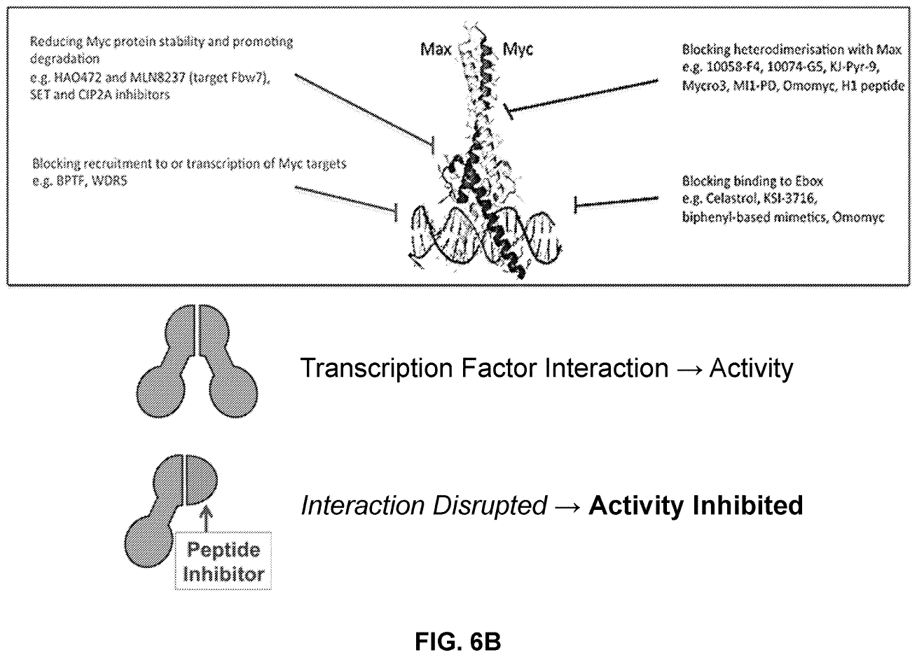

Initial studies focusing on the transcription factor Myc, which is widely dysregulated in human cancers (see, FIG. 6A-6B), was performed as a proof of concept laying the groundwork for generating a more expansive arsenal targeting a wide range of difficult to drug proteins. This example demonstrates exemplary synthesis of monomers and homopolymers comprising sidechains hypothesized to facilitate Myc targeting, inhibition of the PPI between Myc and Max, and/or targeted Myc degradation. The characteristics of the resulting monomers and homopolymers were recorded. FIG. 1A provides a summary schematic of PLP structures containing side chains with different/synergistic functionalities. Note: representative sequences shown, nonexhaustive

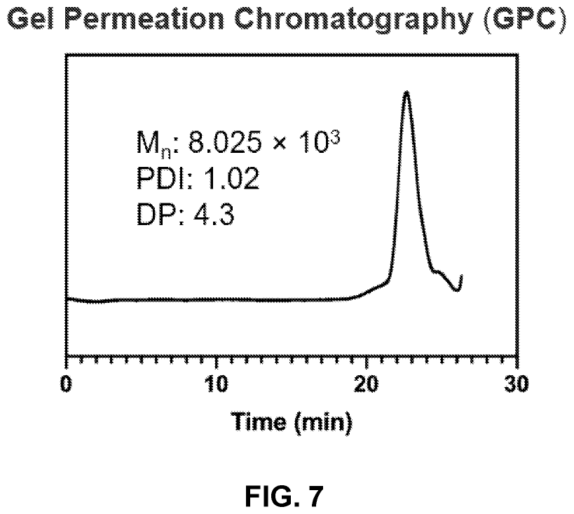

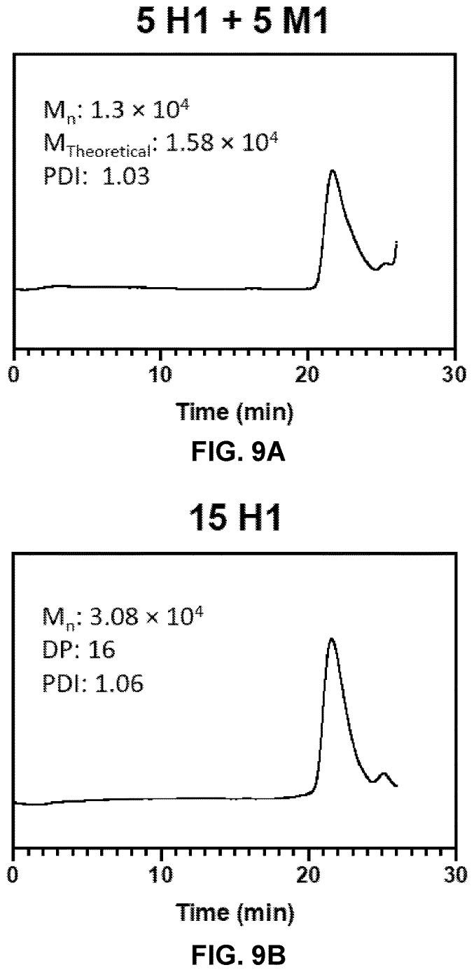

For each of Example 1.1-1.3, peptide sequences were synthesized via Fmoc-SPPS as described in Peptide Synthesis and functionalized into norbomene-monomers on solid support. Reverse phase-high performance liquid chromatography (RP-HPLC) and electrospray ionization (ESI) were used to purify and characterize the synthesized monomers. PLPs were generated via ROMP with predetermined degrees of polymerization (DP) by changing monomer to initiator ratios. In some cases, polymerization kinetics were observed via elongation measurements and, in some cases, resulting DPs, molecular weights and polydispersity were observed via GPC.
Example 1.1—Synthesis of H1 Monomer and Polymerization Results: A previously identified peptide sequence with inhibitory activity against Myc, H1, was generated into PLPs. FIG. 1C demonstrates the representative electrospray ionization (ESI) mass spectrometry of an H1 monomer. FIG. 1D (left) shows HPLC results of the purified H1 monomer (Nor-HI) species demonstrating successful functionalization. FIG. 1E provides the general ROMP mechanism involving reacting the Nor-H1 monomers with a Grubbs catalyst (1.) and an ethyl vinyl ether (2.) to form homopolymers of the desired DP. The conversion rate and pseudo first-order kinetics of H1 homopolymers is depicted in FIG. 1F suggesting successful polymerization. Successful polymerization of a H1-PLP having an average 5 DPs is also demonstrated in the GPC results shown in FIG. 7. Polymerization kinetics of a H1-PLP having 10 DPs is demonstrated in FIG. 1F. Furthermore, GPC results for a H1-PLP having 15 DPs is shown in FIG. 9B.
Example 1.2—Synthesis of Degrading Agent Monomer and Polymerization Results: The RRRG degrader agent was identified as a tetrapeptide capable of degrading a range of protein targets. Accordingly, this degrader agent was generated into PLPs. FIG. 1C (Cont'd) demonstrates the representative electrospray ionization (ESI) mass spectrometry of a RRRG monomer. FIG. 1D (right) shows HPLC results of the purified RRRG norbornenyl monomer (Nor-RRRG) species demonstrating successful functionalization. FIG. 1E provides the general ROMP mechanism involving reacting the Nor-RRRG monomers with a Grubbs catalyst (1.) and an ethyl vinyl ether (2.) to form homopolymers of the desired DP. The conversion rate and pseudo first-order kinetics of HI homopolymers is depicted in FIG. 1F (Cont'd) suggesting successful polymerization.
Example 1.3—Synthesis of Nuclear Localization Peptide Monomer and Polymerization Results: A previously identified native Myc nuclear localization sequence, SEQ ID NO: 18 (PAAKRVKLD) (i.e., “M1”), was selected as the nuclear localization peptide to be incorporated into the HYDRAC PLP platform. An exemplary peptide was synthesized comprising SEQ ID NO: 18 and functionalized into norbomene-monomers on solid support. FIG. 1D (center) shows HPLC results of the purified M1 norbornenyl monomer (Nor-M1) species demonstrating successful functionalization.

### Example 2

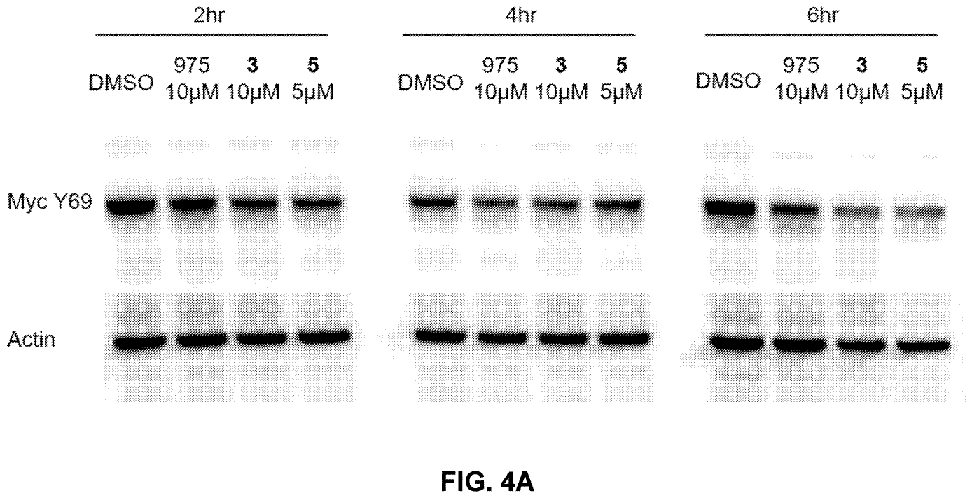

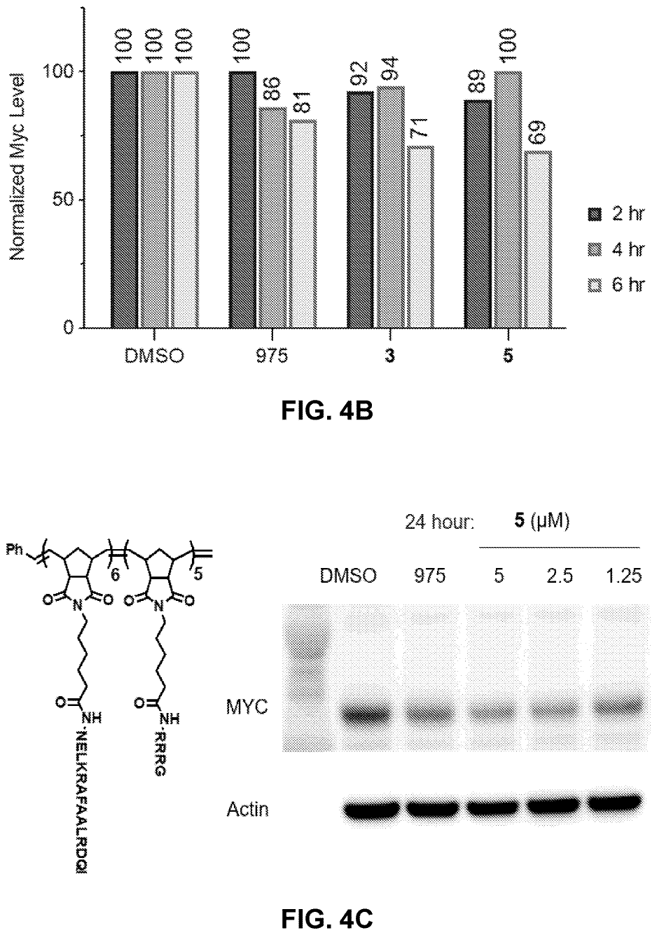

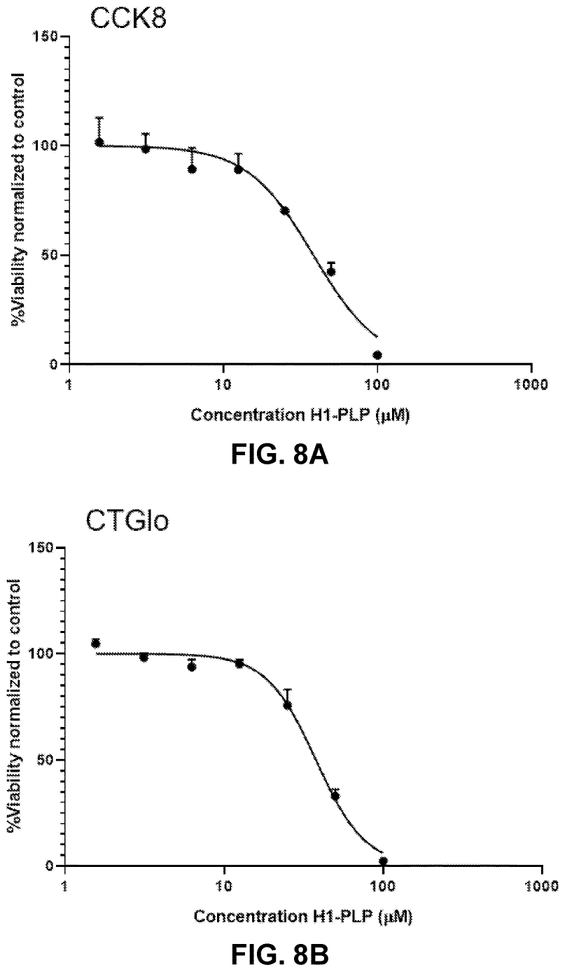

This example provides an exemplary synthesis and associated experimental results of a PLP having a Myc binding peptide as well as a degrader agent, specifically the tetrapeptide (RRRG).
Example 2.1—Methods: This degrader agent, as well as the previously identified Myc binding peptide, H1, were generated into homopolymers as well as block and random (statistical) copolymers.
Example 2.1—Results: FIG. 4C (left) demonstrates an exemplary HYDRAC PLP containing Myc binding peptide H1 paired with the RRRG degrader agent. The conversion rate and pseudo first-order kinetics of H1 homopolymers and RRRG homopolymers as depicted in FIG. 1F and FIG. 1F (Cont'd), respectively, demonstrate similar rates of conversion.
Example 2.2—Methods: To confirm activity in PC-3 cells, two metabolic assays were performed (CCK8 and CTGlo) with a H1-PLP having an average ~5 DPs. In this specific example, the sequence of the Myc binding peptide was SEQ ID 1: NELKRAFAALRDQI, the number average molecular weight (Mₙ) was 1.145×10³ g/mol, the polydispersity index was 1.02, and the average DP was 5.9.
Example 2.2—Results: As demonstrated in FIGS. 8A-8B, the percent viability of PC-3 cells decreased as the concentration of the H1-PLP increased in both the CCK8 assay and the CTGlo assay, which suggests that the H1-PLP is active within PC-3 cells.

### Example 3

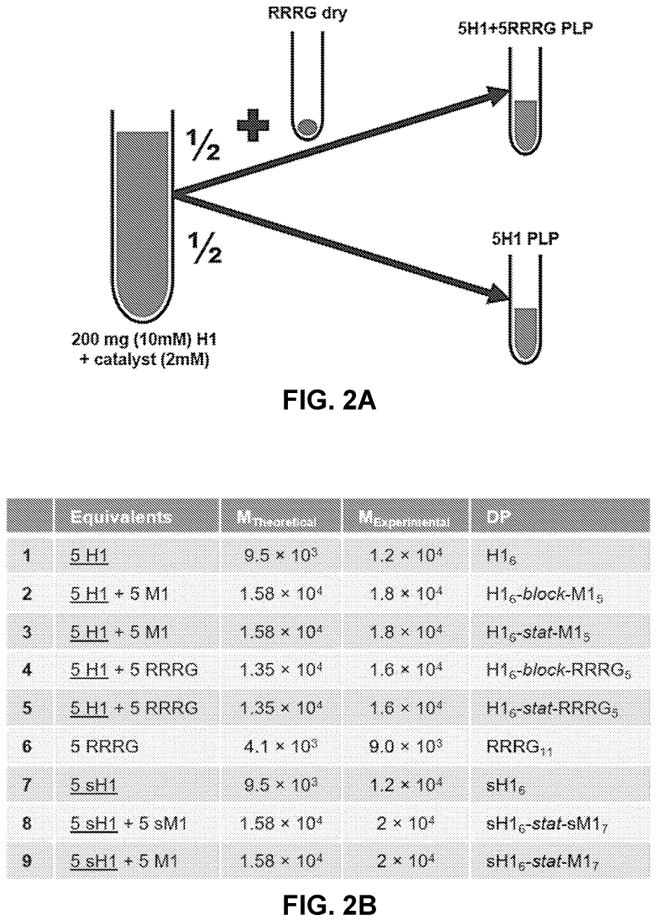

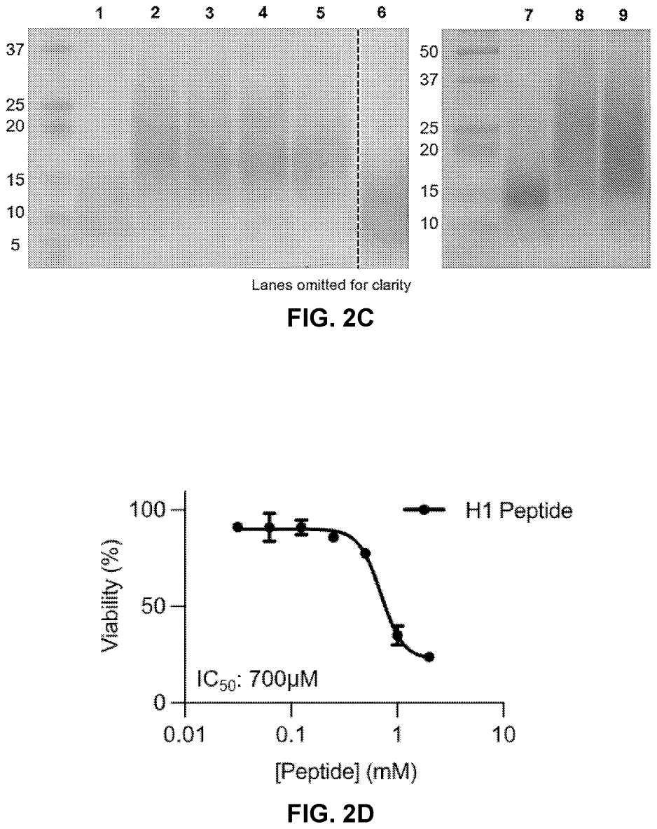

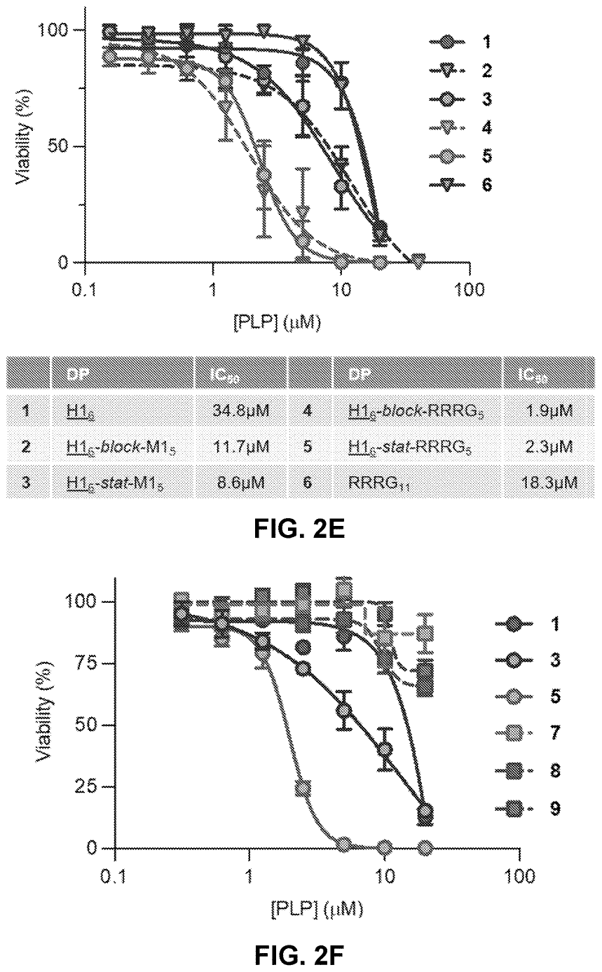

This example evaluates the toxicity of Myc-targeting PLPs in the PC-3 cancer cell line.
Example 3.1—Generation of Block and Statistical Copolymers. A set of PLPs were synthesized and tested for effects on cell viability against the PC-3 prostate cancer cell line. FIG. 2A provides the polymerization setup to generate structures wherein the DP of the first block (H1 in this example) is conserved between daughter constructs. In brief, a batch reaction was started where the desired molar ratios of H1 monomer or scrambled H1 monomer (sHI) to M1 monomer or RRRG monomer (5:1 in this case) were added. For block copolymers, the reaction was left to run to completion before a portion was aliquoted out and the second monomer block was added. For statistical copolymers, the reaction was aliquoted immediately following M1 monomer or RRRG monomer addition.
Example 3.1—Results. FIG. 2B provides the resulting PLPs synthesized as well as the starting equivalents of H1 monomer added to the reaction relative to the M1 monomer or RRRG monomer, the theoretical Mn, and the experimentally determined Mn. The molecular weights were estimated using SDS-PAGE as displayed in FIG. 2C with copolymers having higher DPs displaying higher molecular weights. These results suggest generation of PLPs with conserved amounts of H1 monomer or sHi monomer.
Example 3.2—PC-3 Toxicity Methods. PC-3 prostate cancer cells were cultured via the Cell Culture protocol provided above. PC-3 cells were treated with unpolymerized H1 peptide, the generated block and statistical copolymers, and scrambled controls for 72 hours. Cellular viability was assessed via the Cell Prolferation Assays protocol provided above.
Example 3.2—Results. Cells treated with unpolymerized H1 peptide demonstrated an IC₅₀ (concentration of peptide which exhibited 50% cell viability for PC-3) value of 700 μm (FIG. 2D). PC-3 cells treated with the generated block and statistical copolymers demonstrated IC₅₀ values ranging from 1.9 μM to 34.8 μM, with the H1-R block copolymer demonstrating the lowest IC₅₀ and the H1 homopolymer demonstrating the highest IC₅₀ (FIG. 2E). FIG. 2F provides the results for the effect of PLPs and the scrambled controls on cellular viability in PC-3 cells after the 72 hour treatment. These results revealed that the incorporation of a Myc-binding peptide (in this case, H1) into the PLP platform has a greater impact on PC-3 viability compared to free Myc-binding peptide. Further, the heterobifunctional PLP carrying both a Myc-binding peptide (in this case, HI) and either a nuclear localization peptide (in this case, M1) or a degrader agent (in this case, RRRG) have a more potent impact on PC-3 viability compared to a Myc-binding homopolymer.

### Example 4

This example demonstrates the subcellular localization capabilities of PLPs in the A549 lung adenocarcinoma cell line.
Example 4.1 Methods: A library of PLPs (see table in FIG. 3C showing equivalents, theoretical Mn, and experimentally determined Mn) were synthesized via Peptide Synthesis and Polymerization protocols, including statistical copolymers and RRRG homopolymers and scrambled copolymers (i.e., non-Myc targeted polymers) as negative controls. Each PLP was labeled with Cy5.5 by copolymerizing one equivalent of dye monomer (FIG. 3A) at the end of each reaction. A549 cells were treated with each Cy5.5-labeled PLP for either 2 or 6 hours with compositions at 5 μm Cy5.5. Apparent molecular weights were measured via SDS-PAGE quantification and a Cy5.5 standard curve was generated to determine dye concentration (FIG. 3C).
Example 4.1 Results: Representative confocal images of A549 cells treated for either 2 or 6 hours with Myc-targeted PLPs demonstrate nuclear accumulation, with nuclear accumulation increasing when PLPs incorporated M1 and RRRG, with RRRG showing greater accumulation (FIG. 3B). The representative confocal images of cells treated for 2 hours with either RRRG homopoymers or scrambled copolymers showed minimal, if any, increase in nuclear localization (FIG. 3D). These results suggest that a degrader agent or a nuclear localization peptide incorporated into the PLP platform may improve uptake of the PLP in a cell nucleus.

### Example 5

The following example provides experimental data associated with the reduction of Myc protein levels in samples treated with PLPs containing a Myc-binding peptide copolymerized with either the nuclear localization sequence or degrader agent.
Example 5.1 Methods: PLPs having the equivalents, theoretical Mn, experimentally determined Mn, and compositions of PLPs 3 and 5 of FIG. 2B were synthesized via Peptide Synthesis and Polymerization as described above, where 3: H1₆-stat-M15 and 5: H1₆-stat-RRRG5. A Western Blot Analysis was performed after incubating samples for 2, 4, or 6 hours with PLPs, a DMSO negative control, and a small molecule Myc inhibitor positive control (i.e., “975”). Decreasing doses of PLP 5, 5 μM, 2.5 μM, and 1.25 μM, were then used to treat Myc samples over a 24 hour period and measured by Western BlotAnalysis.
Example 5.1 Results: FIG. 4A provides the western blot results of Myc protein levels following incubation with PLPs and controls at indicated concentrations and incubation times, and quantification of the resulting bands in provided in FIG. 4B. These results demonstrate a decreased level of Myc protein in most samples treated with PLP 3 (H1₆-stat-M1₅) or PLP 5 (H1₆-stat-RRRG₅), with PLP 5 displaying the largest decrease after 6 hours of treatment. Based on these results, PLP 5 was selected to run the 24 hour treatment. FIG. 4C shows the dose response decrease in Myc protein levels where all doses of PLP 5 appear to result in similar or lower Myc protein levels compared to the small molecule Myc inhibitor. The 5 μM dose of PLP 5 appears to display the faintest band, indicating lower Myc protein levels, as compared to the lower doses of PLP 5.

### Example 6

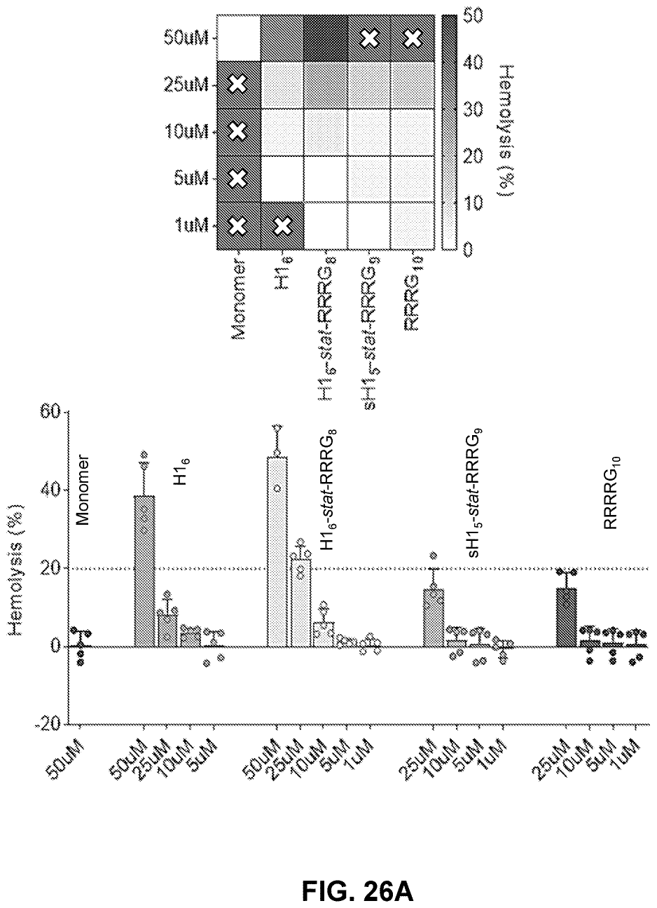

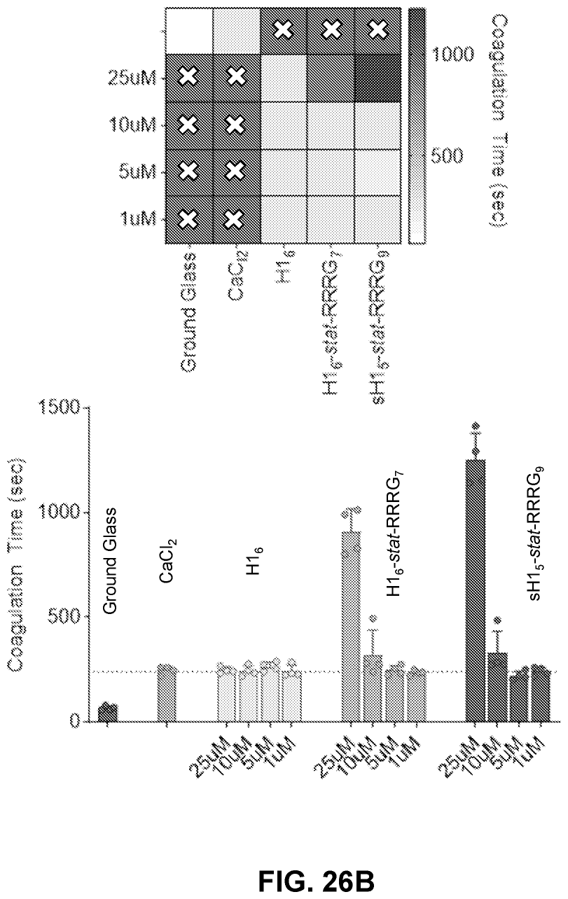

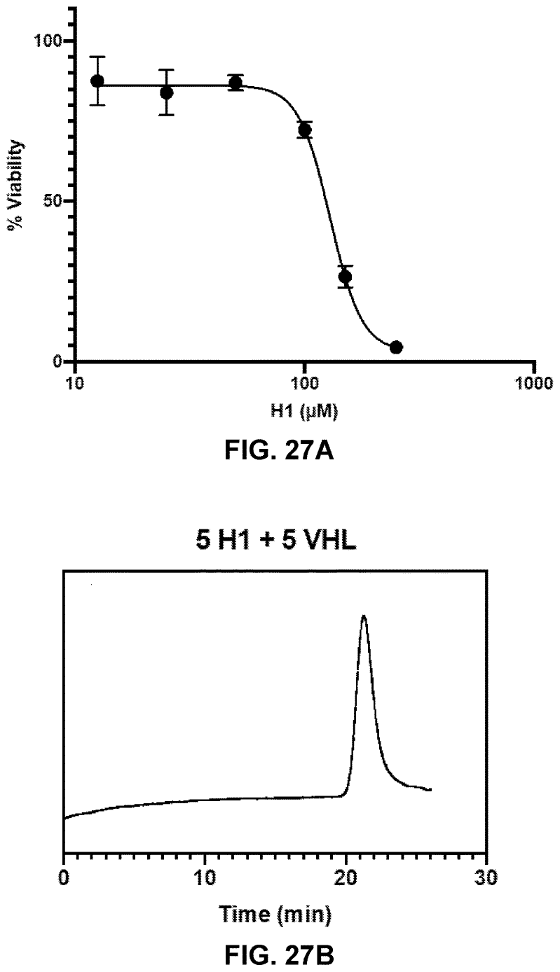

This example provides in vitro biocompatibility results for HYDRAC PLPs having a Myc-binding peptide and a degrader agent.
Example 6.1 Methods: The interaction of a compound with blood will affect immune response, biodistribution, and clearance. The complement system is a key player in the innate immune response, and thus assaying the interaction between the complement system and PLPs serves to assess inherent inflammatory activity. Hemocompatibility of PLPs having the composition: H1₆-stat-RRRG₈ was assessed via activated clotting time (ACT) and hemolytic activity. The H1 monomer, H1₆, RRRG₁₀, and sH1₅-stat-RRRG₉ were also evaluated. Each of the compositions were added to isolated human red blood cells (RBCs) at incremental concentrations from 1 μM to 50 μM for the hemolytic activity assay. For the ACT assay, citrated whole human blood was added to increasing incremental concentrations of the compositions in 1.1 μM CaCl₂) solutions.
Example 6.1 Results: Hemolytic activity of PLPs having H1 (H1₆-stat-RRRG₈ and H1₆) were found to be comparatively higher when administered at high concentrations in view of the controls (FIG. 26A, providing summary data in form of a heat map and chart). At lower concentrations, however, the hemolytic activity among all compositions was comparable. Similarly, in the ACT hemocompatibility assay, the highest concentrations of PLPs having H1 (H1₆-stat-RRRG₈ and H1₆) displayed significantly different coagulations times as compared to the other compositions and controls tested. However, at the lower concentrations, said PLPs did not show as large of a difference in ACT. (FIG. 26B). The results of these in vitro assays suggest that PLPs are hemocompatible at low concentrations.

### Example 7

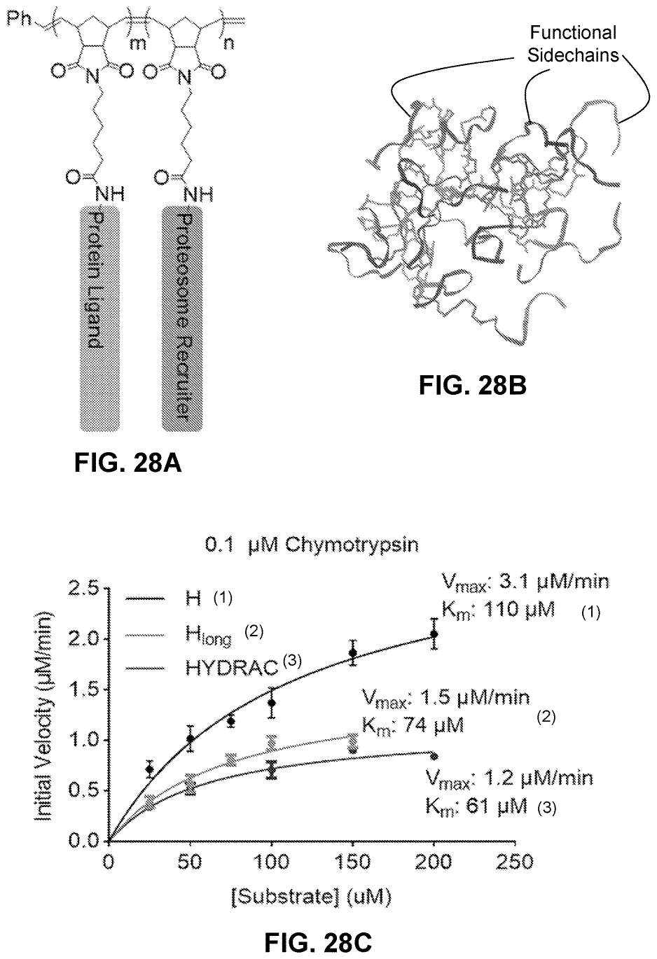

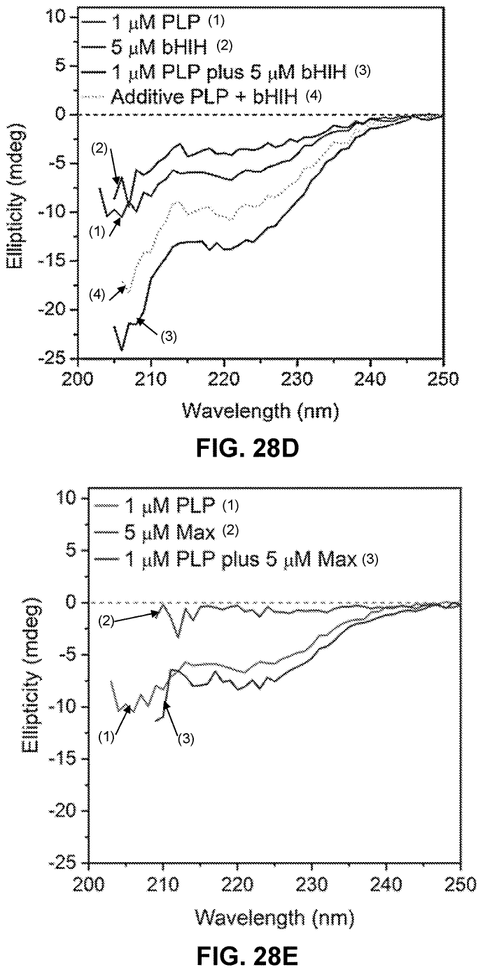

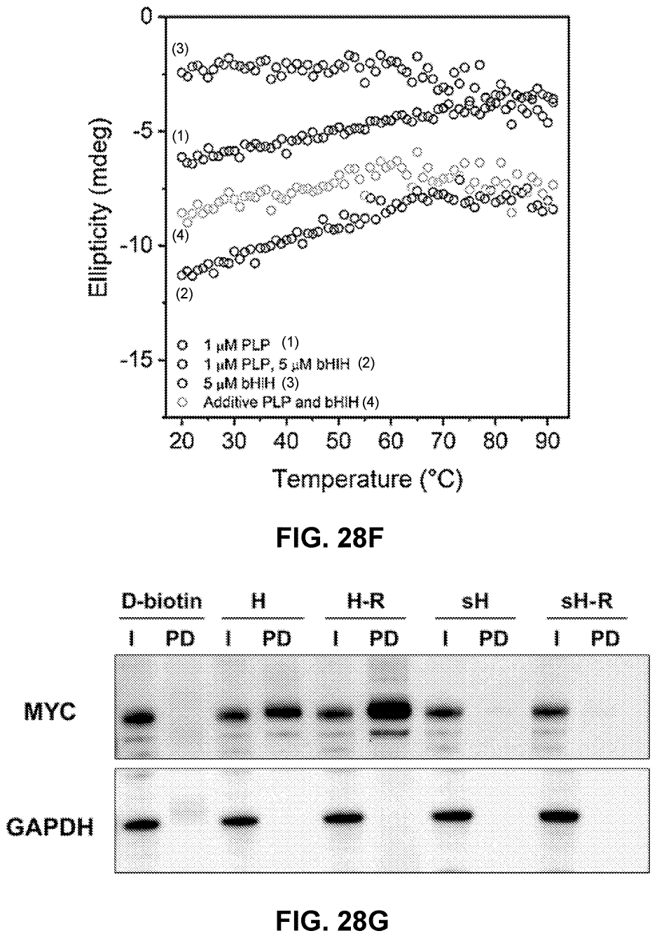

This example provides the design and validation of Myc targeted HYDRAC PLPs comprising a Myc-binding peptide and a degrader agent as exemplified in FIG. 28A.
Example 7.1 Methods: In this example, H1 was used as the Myc-binding pepide and RRRG as the degrader agent. These compounds were synthesized via ring opening metathesis polymerization of peptide containing norbomene monomers as described in Peptide Synthesis and Polymerization, resulting in single chain nanoparticles consisting of a polymer backbone adorned with either Myc- or proteosome-targeting sidechains randomly arranged (i.e., random/statistical copolymers) throughout as follows:

TABLE 1
Polymers synthesized for testing in Example 7.

| FIG.(S) | Label | Mₙ | Composition |
|---|---|---|---|
| 25; 28C | PLP; H | 1.25 × 10⁴ | H1₆ |
| 28C | H_{long} | 2.31 × 10⁴ | H1₁₂ |
| | HYDRAC | 2.20 × 10⁴ | H1₇-stat-RRRG₇ |
| 28D-28F | PLP | 1.75 × 10⁴ | H1₆-stat-RRRG₇ |
| 23-24 | Scrambled PLP | — | sH1₆-stat-RRRG₅ |
| 22 | HYDRAC | 2.00 × 10⁴ | H1₇-stat-RRRG₇ |
| 28G | H | 1.20 × 10⁴ | H1₇-biotin |
| | H-R | 1.80 × 10⁴ | H1₇-stat-RRRG₆- |
| | | | biotin |
| | sH | 1.10 × 10⁴ | sH1₅-biotin |
| | sH-R | 1.80 × 10⁴ | sH1₇-stat-RRRG₆- |
| | | | biotin |

The leftmost column indicates which particular composition was tested in each Figure.
s: scrambled.
stat: statistical copolymer. The subscripts denote the DP for the respective peptides.

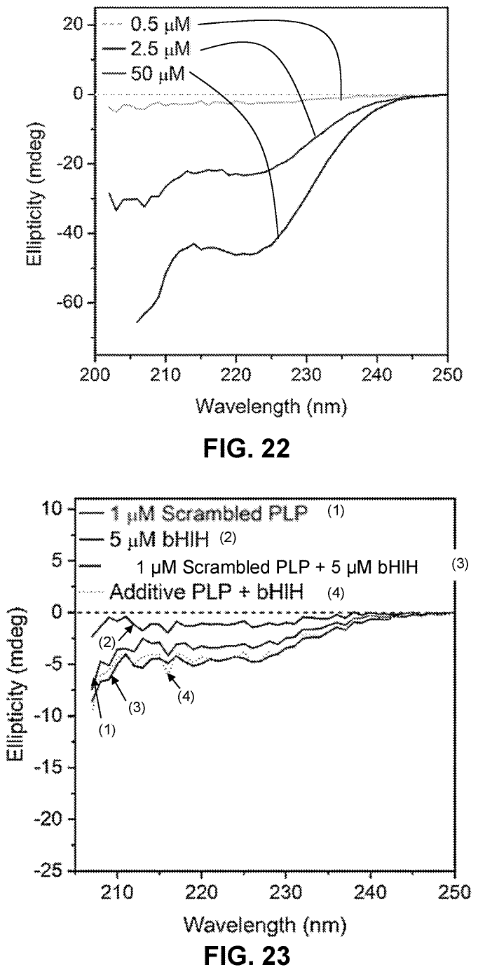

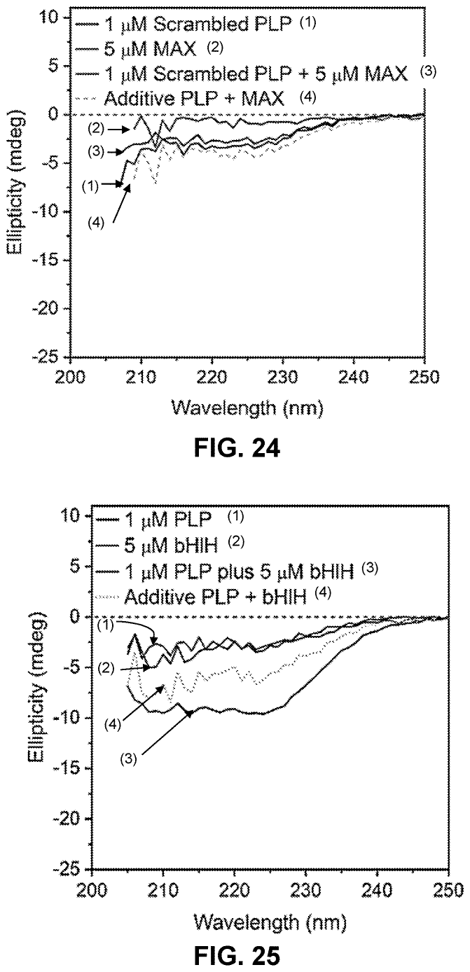

It has been previously reported (Blum et al., Kammeyer et al., and Berger et al., Mussel Adhesive-Inspired Proteomimetic Polymer, Journal of Am. Chem. Soc., 144, 4383-4392 (2022)) that peptide brush polymers with a DP around 15 synthesized in a similar manner maintain payload bioactivity and readily enter cells. As such, initial HYDRAC designs revolved around this molecular size range. In aqueous environments, these nanoparticles fold into globular morphologies reminiscent of native proteins (FIG. 1B, FIG. 28B), with the beneficial emergent property of being highly resistant to proteolytic degradation due to spatial hindrance of enzyme sites. See Berger et al., and Callmann et al., Poly(peptide): Synthesis, Structure, and Function of Peptide-Polymer Amphiphiles and Protein-like Polymers. Accounts of Chemical Research 53, 400-413 (2020), both of which are hereby incorporated by reference. Using chymotrypsin as a model enzyme with a single cleavage site in the H1 sequence, cleavage rates were tracked between compositions using HPLC (FIG. 28C).
Example 7.1 Results: Consistent with previous reports, the DP appeared to influence proteolysis. Polymers containing a higher number of side chains appeared to be more resistant, independent of the specific sequence of side chains (FIG. 28C).
Example 7.2 Methods: In order to assess target engagement of Myc, the ability of HYDRACs to dimerize with either the b-HLH-LZ domain of Myc or full length Max was characterized using a combination of Circular Dichroism (CD) Spectroscopy, as described above, and NMR.
Example 7.2 Results: The CD spectrum of H1-R PLP alone, tested at increasing concentrations of the PLP, resulted in minima around 208 nm and 224 nm for 2.5 μM and 50 μM (FIG. 22), characteristic of a helical homodimer. The lowest concentration tested, 0.5 μM, lacked sufficient pronouncement for characterization. The CD spectrum of another batch of H1-R PLP alone also showed similar minima around 208 and 222 nm, which was comparable to Myc b-HLH-LZ alone (“5 μM bHlH”), as shown in FIG. 28D. In contrast, full length Max showed only a shallow minima, which suggests limited homodimerization (FIG. 28E). A pronounced gain in helical-specific signal intensity was observed only in the experimental mixture of H1-R PLP plus Myc b-HLH-LZ (“1 μM PLP plus 5 μM bHlH”) compared to the arithmetic sum of each component curve (FIG. 28D), suggesting selective Myc/HYDRAC heterodimerization in the low micromolar range versus limited Max/HYDRAC binding (FIG. 28E). Furthermore, the formed Myc/HYDRAC heterodimers were thermodynamically stable up to 90° C. (FIG. 28F). Homopolymers containing just the Myc-targeting H1 sequence (“1 μM PLP”) also showed selective heterodimerization with Myc b-HLH-LZ (FIG. 25), and the scrambled sequence PLP controls lost binding ability with Myc (FIG. 23). And, as expected, the scrambled sequence PLP controls showed little, if any, selective Max binding (FIG. 24).
Example 7.3 Methods: To test for target engagement of H1-containing PLPs, PC-3 cell lysates were treated with DMSO vehicle or biotin-terminated polymer compositions (as indicated in Table 1 above) at 5 μM for H1-R copolymer and 10 μM for all other compositions for 2 h, after which HYDRAC-labeled proteins underwent streptavidin pulldown, elution, separation by SDS-PAGE and blotting for Myc and GAPDH. Details of the protocol are described in In vitro Pull Down Assays and Western Blot protocols.
Example 7.3 Results: Input lysate (“I”) and pulldown (“PD”) lanes are shown in FIG. 28G. Representative blots from n=3 independent experiments are depicted. This data suggests that H1-RRRG PLPs were able to pull down Myc protein from PC-3 cell lysates at a lower concentration compared to H1 homopolymers. Without subscribing to any particular theory, it is believed that these results are likely due to increased electrostatic interactions between the negatively charged protein and the polycationic RRRG degrader agent in the H1-R PLP. Polymers having a sHl sequence (i.e., lacking a functional Myc-targeting sequence), both with and without the accompanying degrader agent, expectedly failed to stain for Myc. Taken together, it is believed that these results validate the ability of HYDRACs to selectively engage Myc protein in cell free conditions through orthogonal methods.

### Example 8

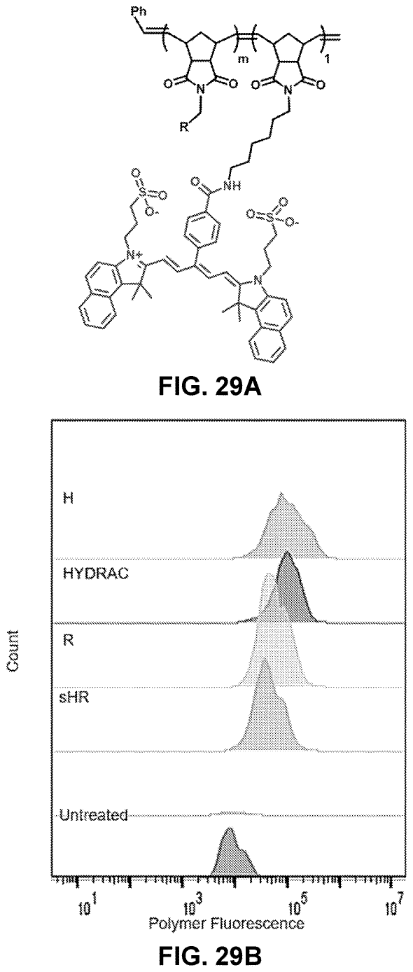

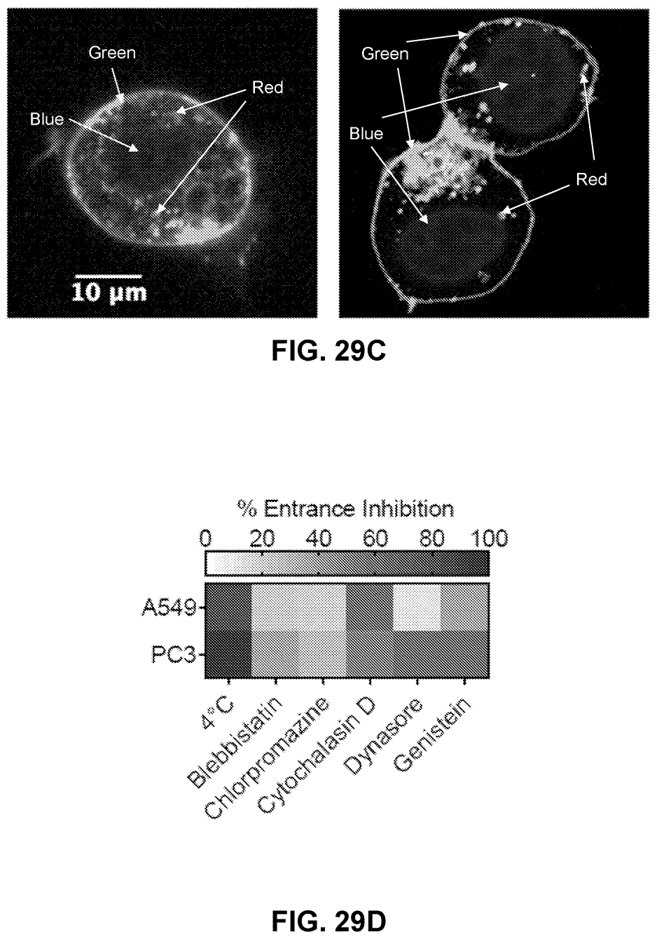

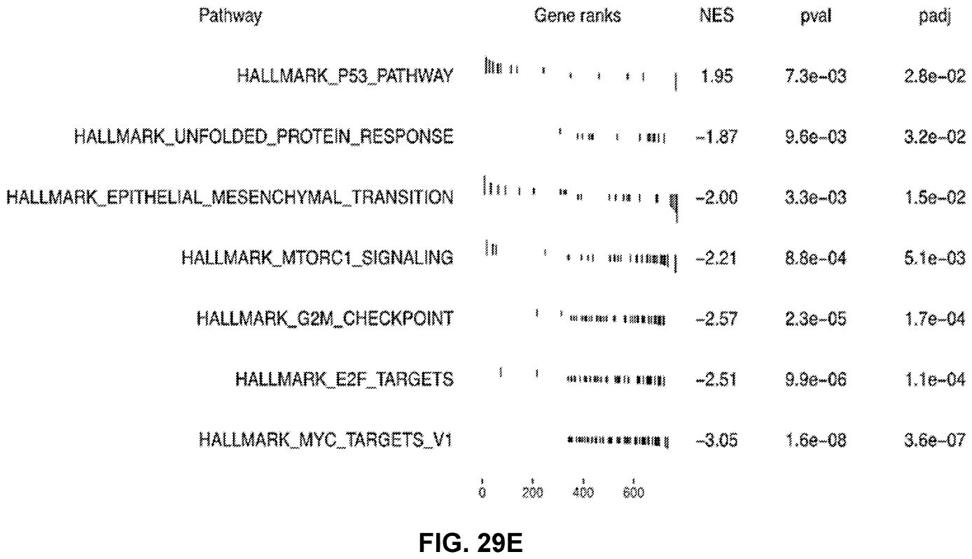

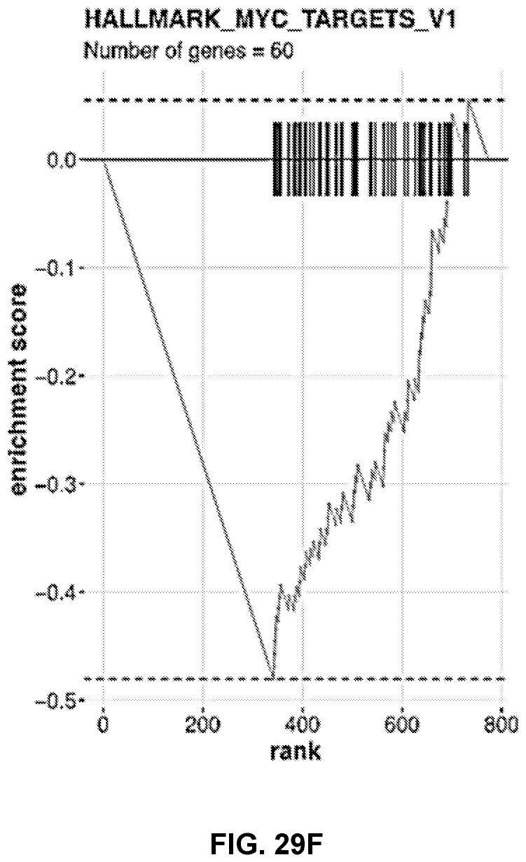

This example supports that Myc-HYDRACs demonstrate localization and targeted transcriptional regulation abilities in PC-3 cells in vitro. In this example, H: H1₆, HYDRAC: H1₆-stat-RRRG₅, R: RRRG₉, and sHR: sH1₆-stat-RRRG₇.
Example 8.1 Methods: A Cy5.5 fluorescent label was added to the end of each PLP composition (H, HYDRAC, R, and sHR) to allow for tracking of internalization in cancer cells. An exemplary representation of a Cy5.5 labeled PLP homopolymer composition is provided in FIG. 29A. To ensure similar fluorescence inputs between treatment groups, a Cy5.5 dosing standard curve was used. PC-3 cancer cells were incubated with H1 and RRRG homopolymers, HYDRACs, or a scrambled copolymer control at a 0.5 μM Cy5.5 concentration for 1 hour in media, trypsinized and washed with heparin to remove surface bound materials, then analyzed by flow cytometrry. To confirm uptake, the PC-3 cells were treated with Cy5.5—H and with Cy5.5-HYDRAC at a 0.5 μM Cy5.5 concentration for 2 hours, stained with WGA-488 and Hoechst 33342, fixed, and imaged by confocal microscopy.
Example 8.1 Results: All PLPs tested showed levels of uptake in PC-3 cells as quantified by flow cytometry fluorescence-activated cell sorting (FACs) (FIG. 29B) with internalization confirmed by confocal microscopy (FIG. 29C). These findings suggest that regardless of the peptides being delivered by the PLP platform, all tested PLP iterations are capable of cellular uptake after 1 hour of treatment. The two confocal images in FIG. 29C provide further evidence that the H1 PLP (left) and the HYDRAC PLP (right) display cellular uptake in PC-3 cells.
Example 8.2 Methods: A combination of uptake pathways, including clathrin/caveolin-mediated endocytosis and macropinocytosis (M) are known to be critical to how both cell penetrating peptides (CPPs) and nanomaterials in general enter cells. For additional information on these uptake pathways, see Koren, E. & Torchilin, V. P. Cell-penetrating peptides: breaking through to the other side. Trends Mol Med 18, 385-393 (2012) and Behzadi, S. et al. Cellular uptake of nanoparticles: journey inside the cell. Chem Soc Rev 46, 4218-4244 (2017). To elucidate the contributions of each of these mechanisms on the uptake of HYDRACs, PC-3 and A549 cells were pretreated with inhibitors of micropinocytosis (100 μM blebbistatin, 10 μM cytochalasin D), clathrin-mediated (1 μM chlorpromazine, 160 μM dynasore), or caveolin-mediated endocytosis (200 μM genistein) for 15 min followed by treatment with 1 μM of HYDRAC-Cy5.5 for 15 min in the presence of the inhibitor, trypsinized, and analyzed by flow cytometry. Percent entrance inhibition was calculated from comparison with vehicle-treated control cells. All cells kept at 37° C. unless otherwise noted.
Example 8.2 Results: The heatmap in FIG. 29D shows summary data from n=3 independent experiments, which suggests that incubation of both PC-3 and A549 cell lines in 4° C. attenuated Cy5.5 signal. This result implies that HYDRACs enter cells largely through adenosine triphosphate-dependent uptake mechanisms. Flow cytometry analysis showed slight variations in the relative importance of each pathway between cell lines, however, micropinocytosis and clathrin-mediated endocytosis appear to play the dominant role in HYDRAC uptake with lesser involvement of caveolin-mediated endocytosis (FIG. 29D), although this pattern is likely cell-line dependent and may require further study.
Example 8.3 Methods: To evaluate whether the PLPs had cellular activity and exerted their effect by altering Myc transcriptional activity, transcriptome sequencing (RNA-Seq) was performed in PC-3 cells treated for 24 hours with 5 μM HYDRAC, comparing transcription-level changes with scramble control or vehicle (DMSO) treated cells.
Example 8.3 Results: Normalized enrichment scores (NES), p values (pval), and adjusted p values (padj) of top gene sets are listed in the gene set enrichment analysis (GSEA) in FIG. 29E. Representative plot of Hallmark Myc signatures enriched in vehicle samples compared to MYC HYDRAC treatment is shown in FIG. 29F. Treatment with HYDRAC led to downregulation of several Myc-driven gene signatures, such as the HALLMARK MYC Targets V1, E2F targets and G2M checkpoint, as revealed by GSEA (FIG. 29E). Notably, the Myc signatures were the most significantly enriched pathways, with the Myc Targets V1 exhibiting the largest negative enrichment score (FIG. 29F). These results provide evidence that the PLP engages Myc protein in vitro and prevents it from binding DNA and enacting its transcriptional program. In sum, the combination of cell uptake and transcriptome results indicate that HYDRACs are able to efficiently enter cells and subsequently exert on-target activity.

### Example 9

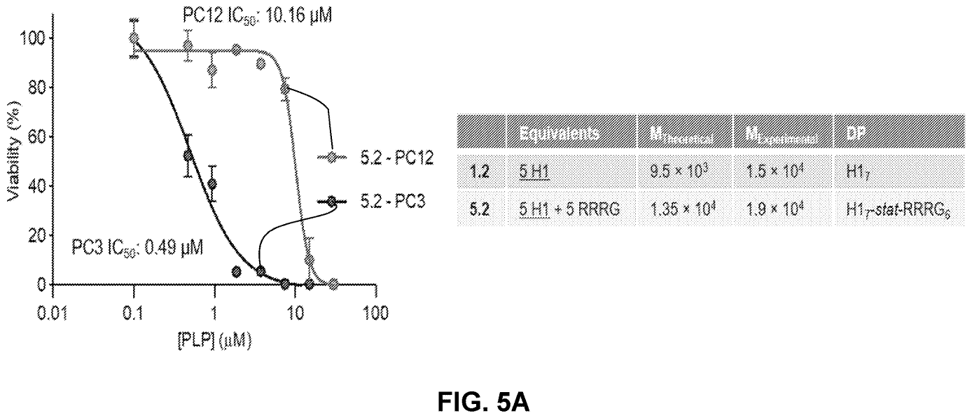

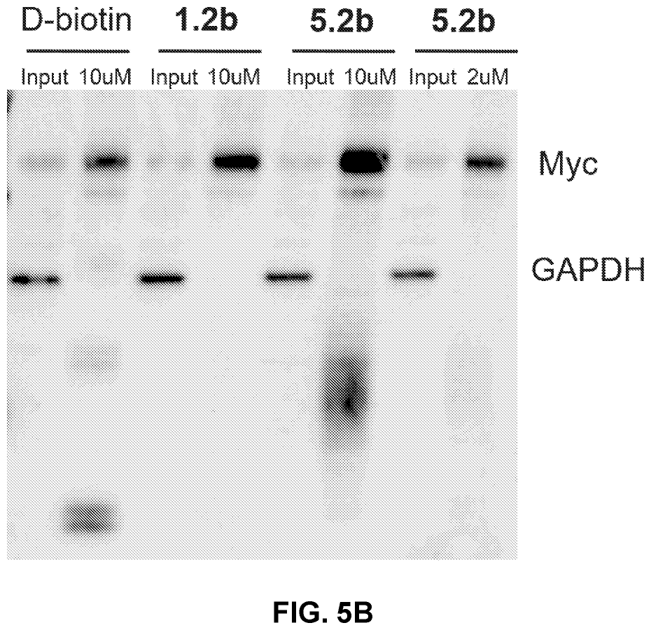

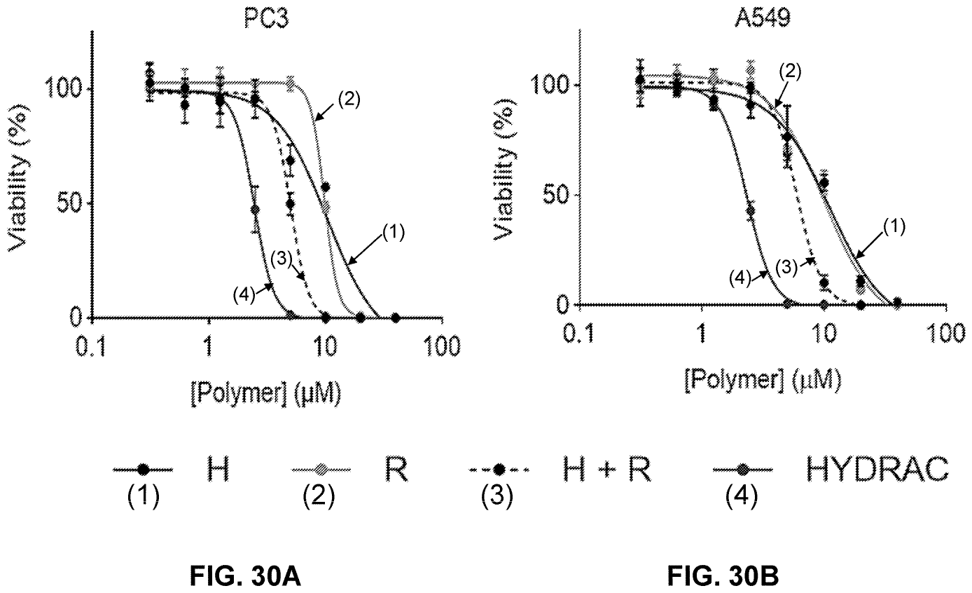

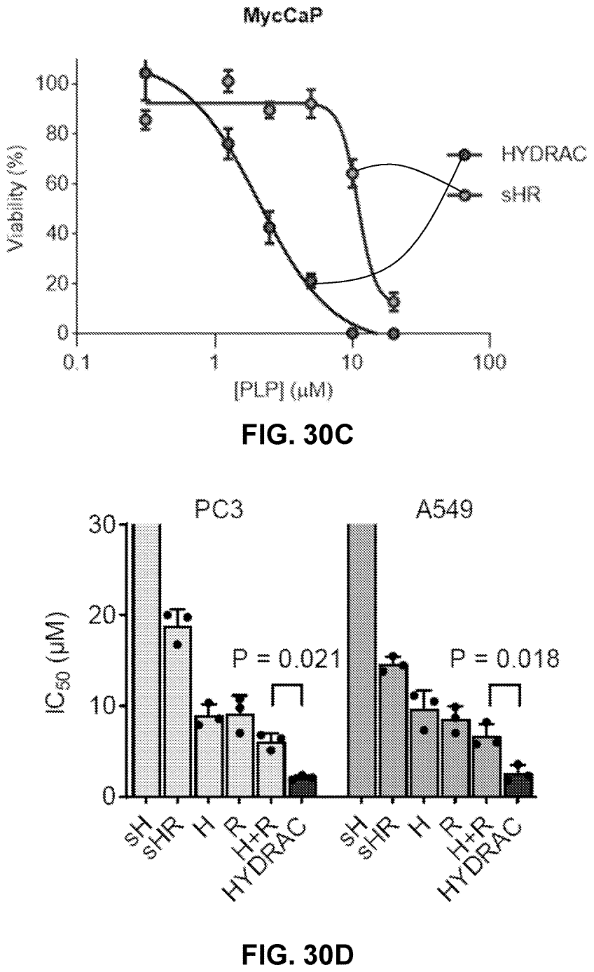

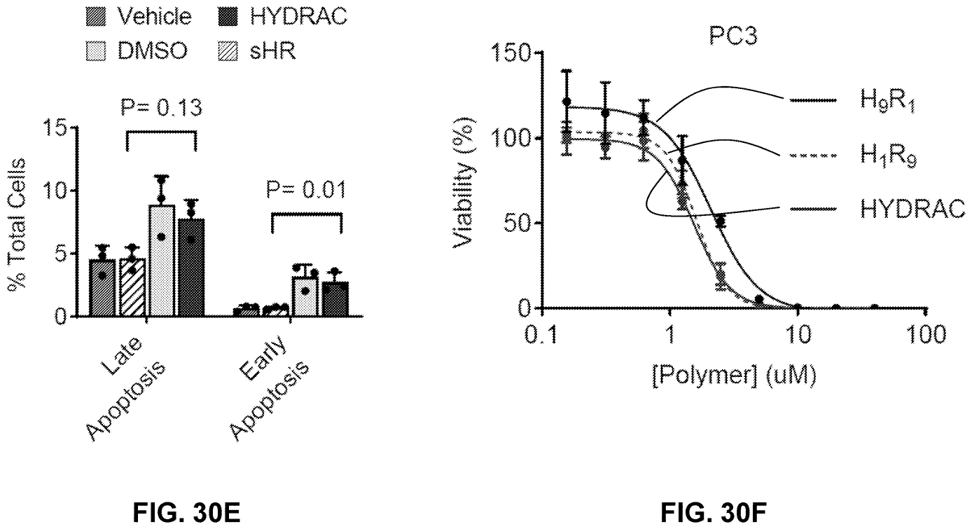

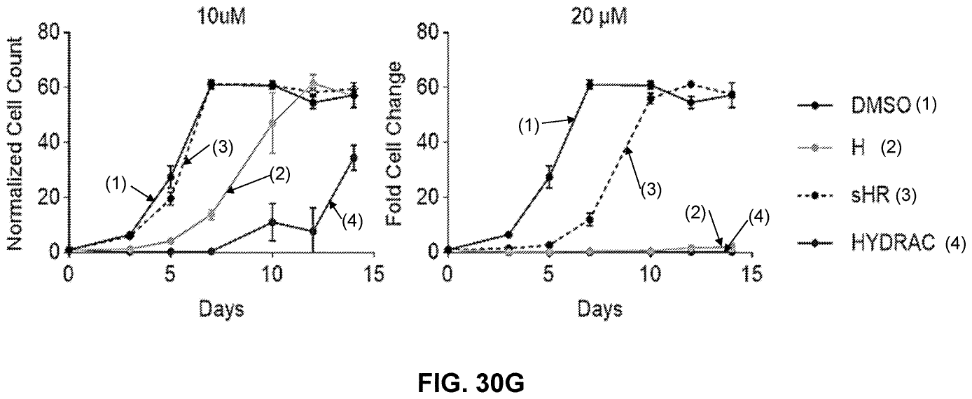

This example provides data supporting toxicity of cancer cells in vitro after treatment with Myc-targeted PLPs. In this example, H: H1₄₋₇, R: RRRG₁₀₋₁₂, H+R: arithmetic sum of H1₄₋₇ and RRRG₁₀₋₁₂, HYDRAC: H1₄₋₆-stat-RRRG₅₋₈, sH: sH1₄₋₆, and sHR: sH1₅₋₆-stat-RRRG₆₋₉.
Example 9.1 Methods: Employing three independent cancer cell lines, PC-3, A549, and MycCaP, toxicity was assessed in vitro following a 72 hour treatment with H, R, HYDRAC, or scrambled controls.
Example 9.1 Results: All cell lines tested showed more susceptibility to HYDRACs compared to the other samples (FIG. 30A: PC-3, FIG. 30B: A549, FIG. 30C: MycCaP). With respect to MycCaP cells, HYDRACs compared to scrambled controls demonstrated a 5 to 10-fold difference in ICso separating the two treatments (FIG. 30C).
Example 9.2 Methods: A library of polymer compositions was next tested in two of the cell lines, PC-3 and A549, to differentiate the contributory effects of each component on the observed toxicity.
Example 9.2 Results: HYDRAC treatment consistently showed lower average IC₅₀ values compared to homopolymers of either the H1 targeting domain or the RRRG degron, alone or in when given together, highlighting the importance of both domains needing to be conjugated together (FIG. 30D). Scrambled controls also showed limited toxicity. Of note, the polymerized version of H1 (H) had a lower IC₅₀ value (~10 μM) compared to the free peptide (~700 μM as discussed in Example 3, FIG. 2D), likely resulting from avidity effects and improved cell uptake. The polymerized RRRG degron (R) itself also showed some inherent toxicity, which is likely from off-target effects, although at a much lower level compared to the HYDRAC composition (FIG. 30D). HYDRAC treated PC-3 cells also showed higher levels of apoptosis compared to scramble controls, as assessed by Annexin V/PI staining (FIG. 30E), adding credence to the evidence that cancer cells are susceptible to HYDRAC therapy.
Example 9.3 Methods: Building upon this HYDRAC composition (1H1: 1RRRG), various H1 to RRRG ratios in the PLP were explored to determine whether efficacy could be improved. PC-3 cells treated with the HYDRAC composition were compared to cells treated with either a 1:9 (H₁R₉) or 9:1 (H₉R₁) H1:RRRG ratio.
Example 9.3 Results: The ratios tested showed similar levels of cellular toxicity (FIG. 30F). Given the similar toxicity levels, no further ratio optimizations were tested at this time.
Example 9.4 Methods: To further explore whether the observed toxicity effects were on-target, PLPs having the equivalents, theoretical Mn, experimentally determined Mₙ, and compositions of PLPs 1.2 and 5.2 in the table in FIG. 5A (1.2: H1₇ and 5.2: H1₇-stat-RRRG₆) were used to treat the PC-3 and PC-12 Pheochromocytoma (which lack the Myc dimerization partner Max) cell lines. Cells were treated with PLP 5.2 for 3 days and cellular viability was recorded. Both PLPs were evaluated for their ability to pull down Myc from 20 PC-3 cell lysate via theIn vitro Pull Down Assay protocol after streptavidin beads of PC-3 cell lysate were treated with 10 μM or 2 μM of the biotinylated PLPs or biotinylated DMSO as a negative control.
Example 9.4 Results: A significantly smaller concentration of PLP 5.2 was required to decrease PC-3 viability (ICsₒ of 0.49 μM) than the concentration required to decrease PC-12 viability (ICsₒ of 10.1₆ μM) as shown in FIG. 5A. This differential viability suggests observed toxicity in PC-3 cells was on-target via Myc inhibition. Further, both the H1 homopolymer (PLP 1.2) and the H1-RRRG statistical copolymer (PLP 5.2) were capable of direct endogenous binding to Myc protein as indicated by the gel in FIG. 5B.
Example 9.5 Methods: Since HYDRACs appeared to resist enzymatic degradation resistance in the model enzyme system described in Example 8.2 and FIG. 29D, HYDRACs were evaluated to examine long-acting effects in vitro. To assess this. A549 cells were incubated with a single treatment of either 10 or 20 μM HYDRAC or control polymer compositions and tracked over two weeks.
Example 9.5 Results: Both H homopolymer and HYDRAC treatments appeared to suppress cell growth in the short term, with HYDRAC treated cell counts not recovering until 2 weeks post treatment, and then at a slower growth rate (FIG. 30G). These results correlate well with trends observed in both the enzymatic degradation and cellular toxicity studies discussed above. Without subscribing to any particular theory, it is believed these results can be attributed to the combination of HYDRACs being both longer circulating and more potent compared to H homopolymers. Scrambled controls, meanwhile, only mildly retarded cell proliferation at a single treatment of 20 μM (FIG. 30G), consistent with the lower calculated IC₅₀ values (FIG. 30D).

### Example 10

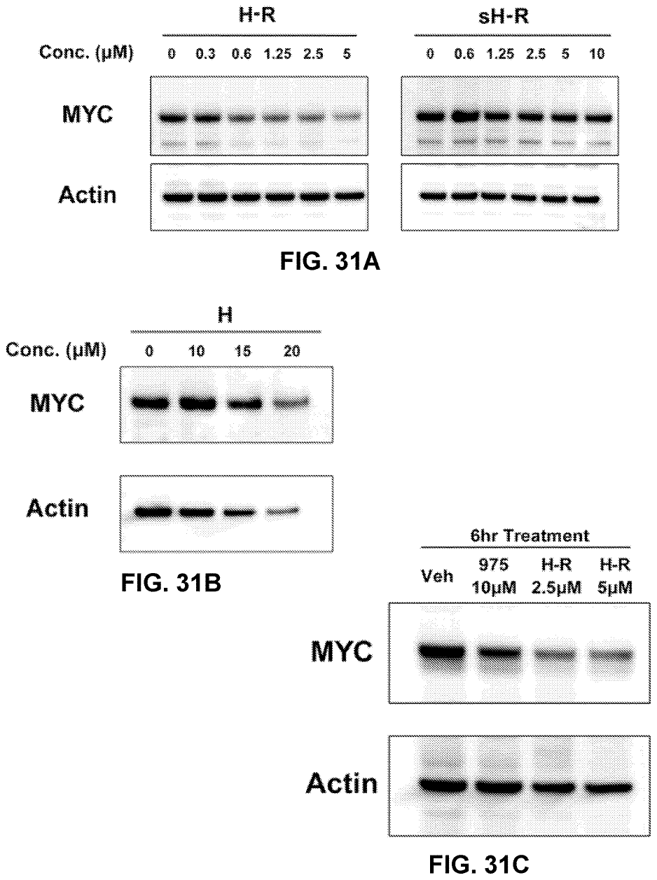

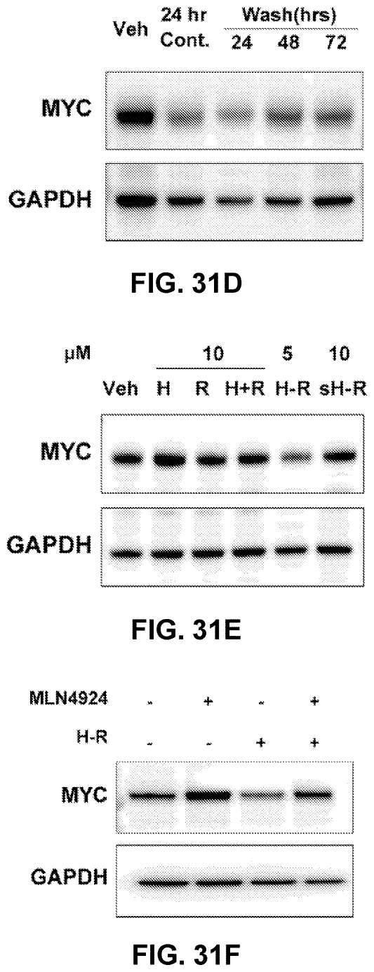

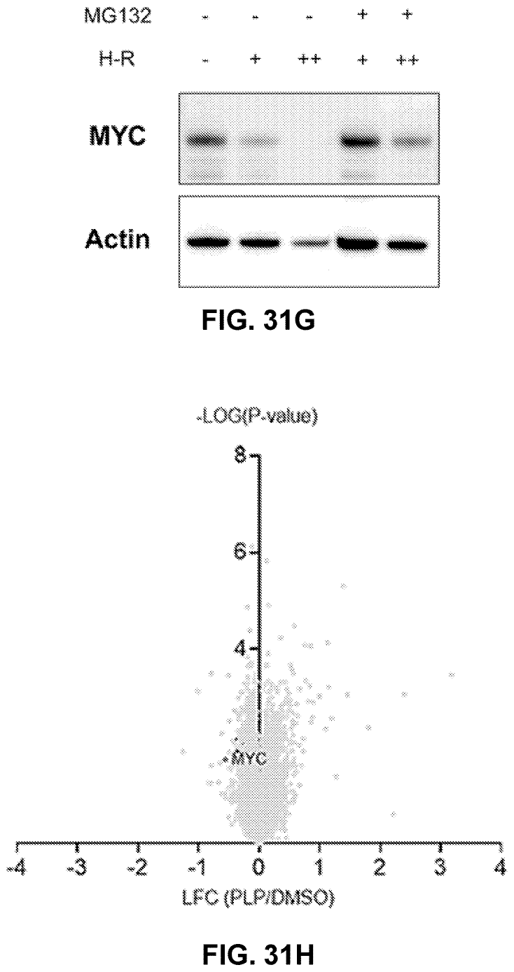

Based on the results from Example 9 suggesting induced on-target effects on cancer cell growth, the following example explored the ability of these constructs to selectively degrade endogenous target protein. In the following example, H: H1₆, R: RRRG₁₂, H+R: arithmetic sum of H1₄₋₇ and RRRG₁₀₋₁₂, H-R: H1₄₋₆-stat-RRRG₅₋₇, Veh: 3:1 DMSO:H₂O (0.3% final concentration), and 975: small molecule Myc inhibitor.
Example 10.1 Methods: To determine whether HYDRACs exhibited similar E3-ligase recruiting abilities as PROTACs, cells were treated for 24 h with HYDRACs (H-R) or a vehicle control prior to being rinsed with PBS to washout extracellular compound, replated into fresh media, and followed for an additional 24, 48, or 72 h.
Example 10.1 Results: HYDRAC (H-R) treated cells showed sustained Myc suppression up to 72 h post washout (FIG. 31D). This observation could partially be attributed to the long-circulating nature of HYDRACs, but is also consistent with a catalytic mode of action, as otherwise the rapid turnover of Myc would be expected to quickly exhaust the small amount of internalized HYDRACs.
Example 10.2 Methods: Endogenous Myc is partially regulated through phosphorylation at threonine 58 (pT58), primed by a phosphorylation cascade initiated by several kinases, ultimately leading to E3 ubiquitin ligase mediated degradation, aspects of which are explained in Sears et al., Multiple Ras-dependent Phosphorylation Pathways Regulate Myc Protein Stability, Genes Dev 14, 2501-2514 (2000), and Zhou et al., Regulation Mechanism of Fbxw7-related Signaling Pathways (Review), Oncol Rep 34, 2215-2224 (2015), both of which are hereby incorporated by reference. Using a MYC^{T58A} mutant cell line, HYDRAC treatment was used to evaluate whether it alters Myc stability through this mechanism. As inhibition of Myc also directly leads to mild suppressions of protein levels, a small-molecule MYC inhibitor (MYCi975) was included as a comparative control. PC-3 MYC^{T58A} cells were treated for 6 h with the 975 Myc inhibitor as well as both 2.5 and 5 μM of HYDRACs.
Example 10.2 Results: Western blot results showed significant decreases in MYC^{T58A} levels, evidence that HYDRAC activity acts through non-endogenous degradation pathways (FIG. 31C). MYCi975 treatment meanwhile showed only minor changes in mutant MYC levels (FIG. 31C).
Example 10.3 Methods: To validate western blot data on Myc degradation and assess proteome-wide perturbations, an unbiased tandem mass tag (TMT)-based quantitative proteomic analysis was performed on HYDRAC treated PC-3 cells.
Example 10.3 Results: MYC levels were detectably decreased by mass spec (FIG. 31H).
Example 10.4 Methods: Degradation of endogenous Myc protein in PC-3 cells was explored by incubating cells for 24 hours with H, H-R, or the negative control, sH-R, at increasing concentrations of PLPs. Additionally, an experiment incorporating a co-mixture of H and R (H+R) and appropriate controls was also performed to evaluate the relative degradation of endogenous Myc protein. In this experiment, PC-3 cells were incubated for 24 hours with 10 μM of each PLP or control (other than H-R, tested at a concentration of 5 μM).
Example 10.4 Results: H homopolymers lacking the RRRG degrader agent failed show reduced endogenous Myc levels up to 20 μM, at which point cellular toxicity effects started appearing (FIG. 31B). In contrast, Myc-targeted HYDRACs (H-R) show noticeably reduced Myc protein bands on Western Blot at doses as low as 0. μM, with non-targeted polymers with the degrader agent (sH-R) showing marginal, if any, protein level reductions, even at nearly 20× the dose (FIG. 31A).
Example 10.5 Methods: Taking 5 μM as the HYDRAC treatment dose with distinct Myc degradation, various control polymer compositions were compared at double the dose.
Example 10.5 Results: Neither treatment with either domain alone, combination treatment with homopolymers of both domains unlinked, or a scrambled targeting sequence control showed appreciable Myc degradation (FIG. 31E), highlighting the potential importance of linking both domains on a HYDRAC. These observed changes in Myc levels were found to occur on the protein-level as relative Myc mRNA remained unchanged following HYDRAC treatment.
Example 10.6 Methods: PC-3 cells were treated with HYDRACs in the presence or absence of proteosome and neddylation inhibitor (MG132 or MLN4924 respectively) to elucidate pathways involved in the observed protein degradation. In these experiments, PC-3 cells were incubated with 5 μM or 10 μM of H-R for 24 hours in the presence or absence of each inhibitor.
Example 10.6 Results: Pretreatment with either inhibitor (FIG. 31F—MLN4924; FIG. 31G —MG132) rescued MYC protein levels, suggesting HYDRAC activity is dependent on both the proteasome and Cullin-RING ubiquitin ligases. Additionally, HYDRAC PLP-induced Myc degradation was reduced when in the presence of proteasome inhibitor MG132 during incubation (FIG. 31G). This result suggests the mechanism utilizes proteasome-mediated degradation, as predicted due to the presence of the RRRG degron sequence.

### Example 11

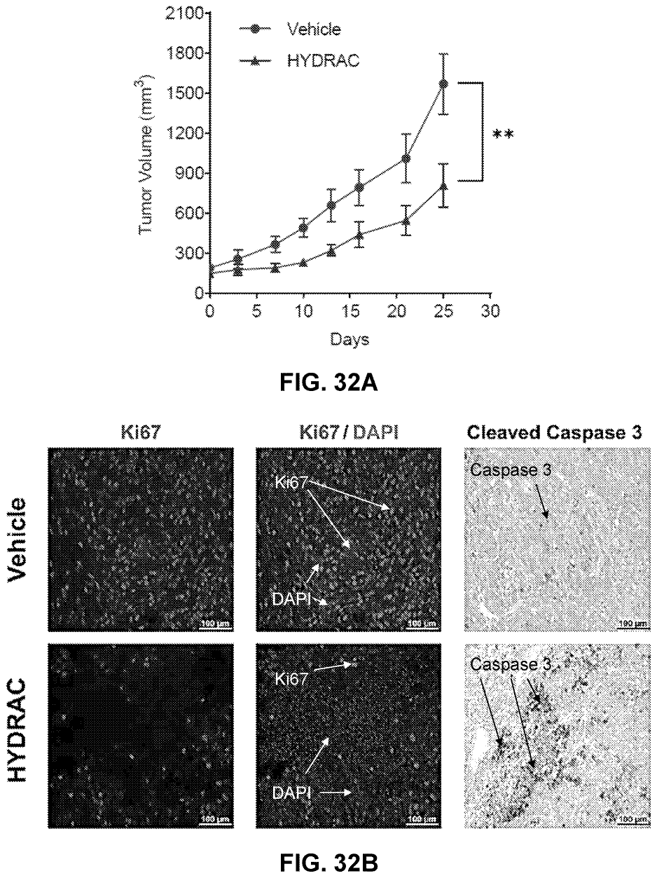

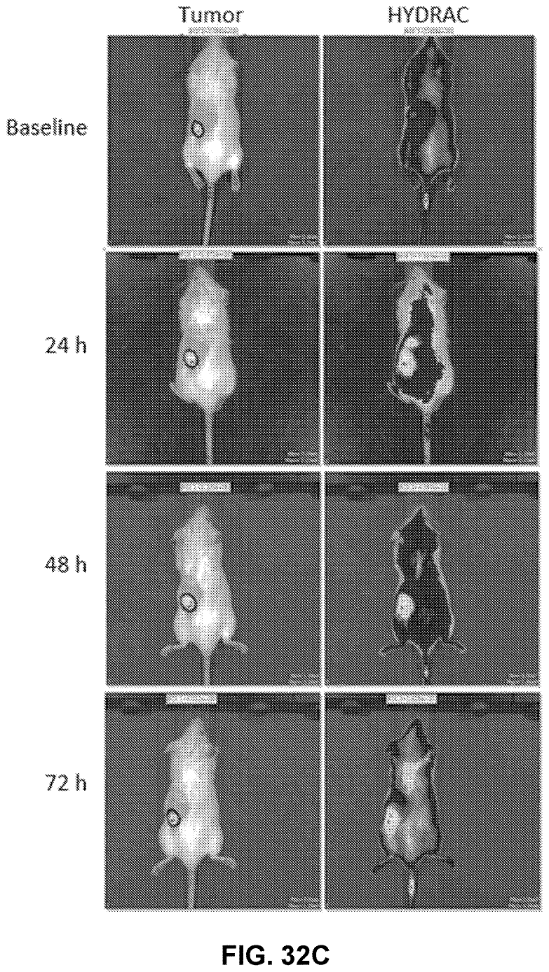

This example provides an assessment of Myc-targeted PLPs in vivo. In the following example, HYDRAC: H1₆-stat-RRRG7.
Example 11.1 Methods: First, to inform dosing strategy going forward, healthy mice were injected intravenously (IV) with increasing doses of HYDRACs to determine maximum tolerated dose (MTD) with 40 mg/kg determined to be the single dose MTD and 25 mg/kg for repeat dosing. A Luc-MV4-11 tumor model paired with Cy5.5 labeled-HYDRACs was then implemented to simultaneously monitor both the location of tumor cells and the trafficking of HYDRACs following a single IV or Intraperitoneal (IP) injection at 25 mg/kg.
Example 11.1 Results: Notable accumulation of HYDRAC-Cy5.5 was observed in the tumor up to 72 h following a single IP administration (FIG. 32C), which was absent in mice injected IV (not shown). Without adopting any particular theory, it is believed that this discrepancy likely stemmed from the RRRG degron exhibiting observed hemolytic activity following IV injection, leading to damage at the injection site which decreases the subsequent amount of drug entering circulation.
Example 11.2 Methods: The in vivo study was assessed in mice bearing established MycCaP tumors/MycCaP cell line, a mouse prostate cancer line expressing human Myc under the control of the androgen receptor (AR), which was previously validated to be sensitive to HYDRAC treatment (Example 9.1, FIG. 30C). The mice were treated with 25 mg/kg HYDRACs dosed three times per week via intraperitoneal (IP) administration. Immunofluorescent (IF) staining and immunohistochemistry (IHC) of tumors excised at day 25 post implantation was subsequently performed for analysis.
Example 11.2 Results: Treatment with 25 mg/kg HYDRACs resulted in significantly suppressed tumor growth (FIG. 32A). FIG. 32B provides representative images of Ki67 marker of proliferation levels assessed by IF and cleaved Caspase-3 levels assessed by IHC in the tumor tissue from the study in FIG. 32A. (scale bar, 100 μM). These images showed reductions in proliferative cells with high levels of cleaved caspase-3 in HYDRAC treated animals (FIG. 32B). These results suggest that the HYDRAC has satisfactory bioavailability and displays stability in plasma long enough to establish effective tumoral localization. The combination of selective tumor site accumulation following IP administration paired with high levels of HYDRAC-induced anti-proliferative effects underscore the therapeutic potential of this platform technology.

### Example 12

This example provides evidence supporting the generalizability of the PLP platform.
Example 12.1 Methods: To capitalize on the capability of PLPs to carry payloads with different sequences of complementary functionalities, peptide and small molecule side chains that recruit cellular degradation machinery were incorporated for the targeted degradation of difficult to drug proteins. A library was generated using an E3 ligase, VHL, by functionalizing and polymerizing VHL into the PLP platform with H1 (H15-stat-VHL5) as well as with both H1 and Ml (H15-stat-M15-stat-VHL5). The resulting PLPs were characterized by GPC, and evaluated for impact on PC-3 cellular viability.

### Example 12.2 Results

TABLE 2
Certain VHL Polymers.

| FIG. | Mₙ | M_{Theoretical} | Composition | IC₅₀ (μM) - PC-3 | PDI |
|---|---|---|---|---|---|
| 27A | — | — | Unpolymerized VHL | 129.1 | N/A |
| 27A-27B | 2.2 × 10⁴ | 1.5 × 10³ | H1₅-stat-VHL₅ | 12.9 | 1.007 |
| 15A; 16A | 2.26 × 10⁴ | 2.08 × 10⁴ | H1₅-stat-M1₅-stat- | 23.1 | 1.01 |
| | | | VHL₅ | | |

stat: statistical copolymer.
The subscripts denote the DP for the respective peptides.
For cells marked “—” data is not shown in the associated figures.

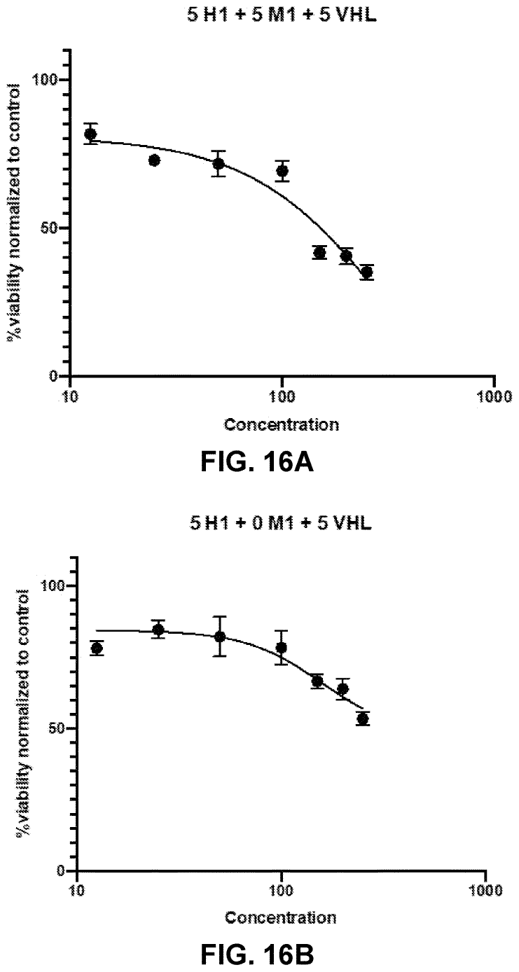

The combined results from Table 2 suggest the successful integration of VHL in the PLP platform. Additional supporting data in FIGS. 16A-16B provides PC-3 toxicity data when treated with PLPs incorporating VHL, with (FIG. 16A) or without (FIG. 16B) nuclear localization agent.
Example 12.2 Methods: VHL and two additional E3 ligases, KEAP1 and thalidomide, were explored for integration into the HYDRAC PLP platform. The ability of the copolymer to induce targeted protein degradation was assessed by utilizing the same H1 (Myc-binding peptide) in the above-described examples. The following compositions were tested:

TABLE 3
Polymers synthesized for testing in Example 12.

| Label | Mₙ | Composition | IC₅₀ (μM) - PC-3 |
|---|---|---|---|
| VHL | 1.21 × 10⁴ | H1₄-stat-VHL₄ | 8.7 |
| KEAP | 1.80 × 10⁴ | H1₅-stat-KEAP₅ | 12.2 |
| Thal1 | 2.40 × 10⁴ | H1₁₅-stat-Thalidomide₁ | 6.5 |
| Thal2 | 2.10 × 10⁴ | H1₁₀-stat-Thalidomide₂ | 36.1 |

stat: statistical copolymer.
The subscripts denote the DP for the respective peptides or small molecules.

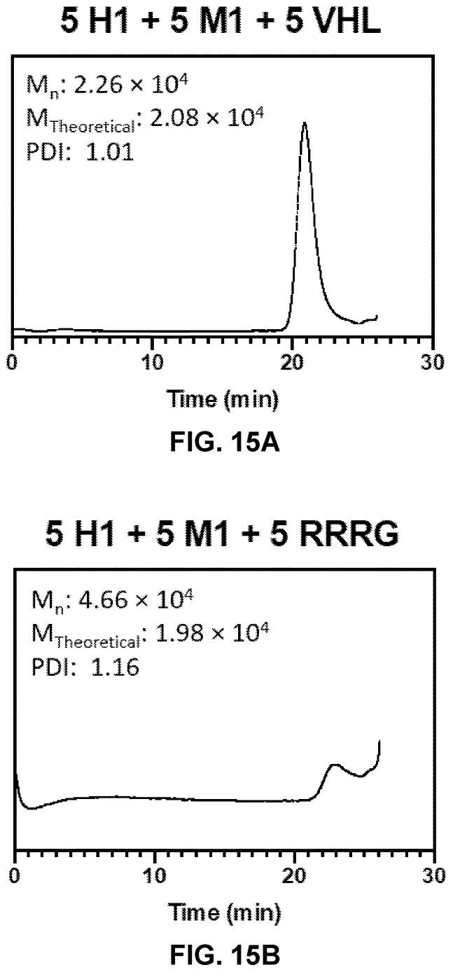

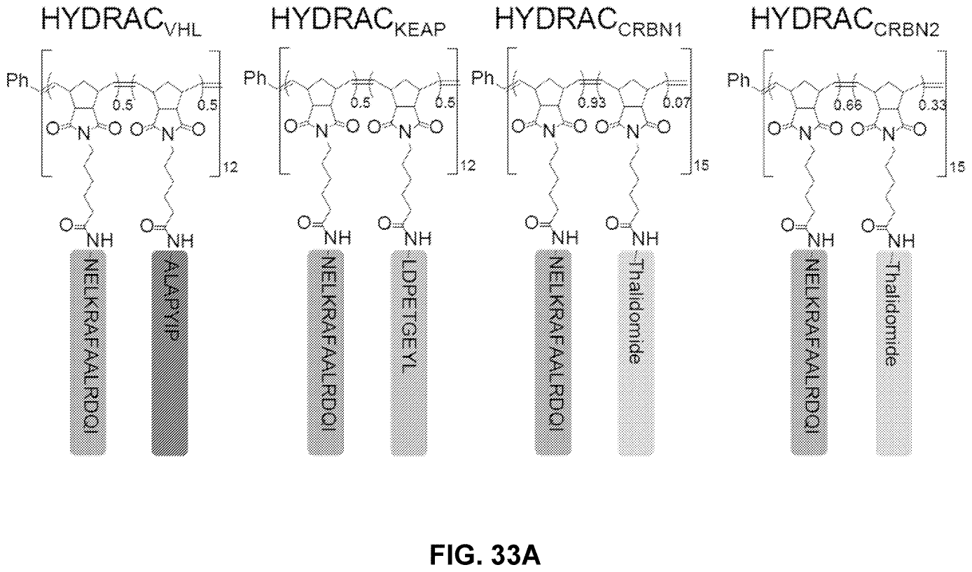

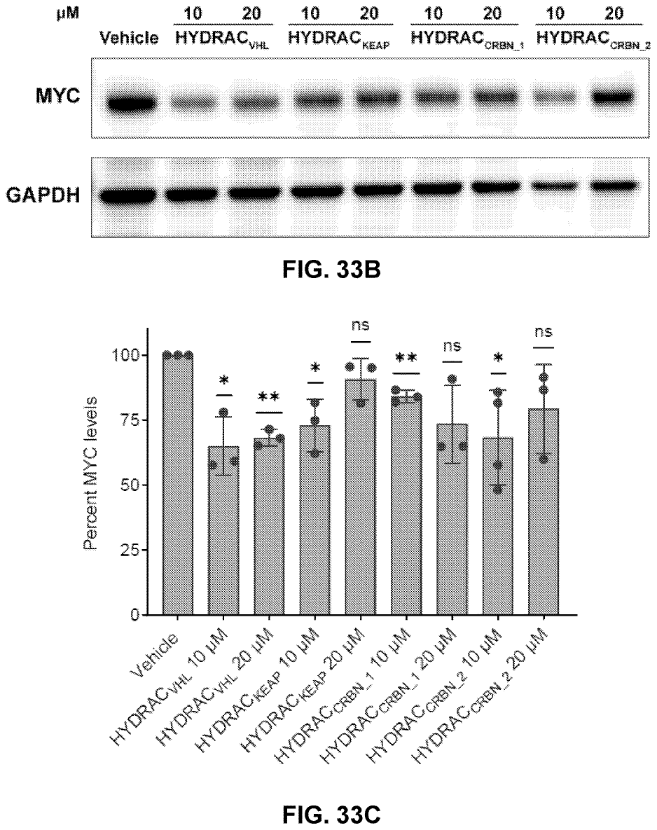

Each of the compositions in Table 3 was incubated with PC-3 cells for 24 hours at concentrations ranging from 10 μM to 20 μM. Since the resulting PLP was intended to target Myc for degradation by the proteosome, the samples were subjected to Western blotting analysis as well as a cell proliferation assay for evaluation.
Example 12.2 Results: FIG. 33A: Structures of the four Myc-targeting HYDRACs incorporating three different E3 ligase recruiting peptides or small molecule. The cell proliferation assay results (graphs not shown) provided the estimated IC₅₀ values for each composition in Table 3 above. Representative western blot (FIG. 33B) and quantification (FIG. 33C) of endogenous MYC protein levels after treatment of PC-3 cells for 24 hours with indicated polymer compositions and concentrations. Data providing a direct comparison of VHL-PLPs and RRRG-PLPs is provided in FIGS. 15A-15B, which portrays GPC graphs comparing heterofunctional PLPs with VHL as degrader agent (FIG. 15A) and RRRG as degrader agent (FIG. 15B).

### Example 13

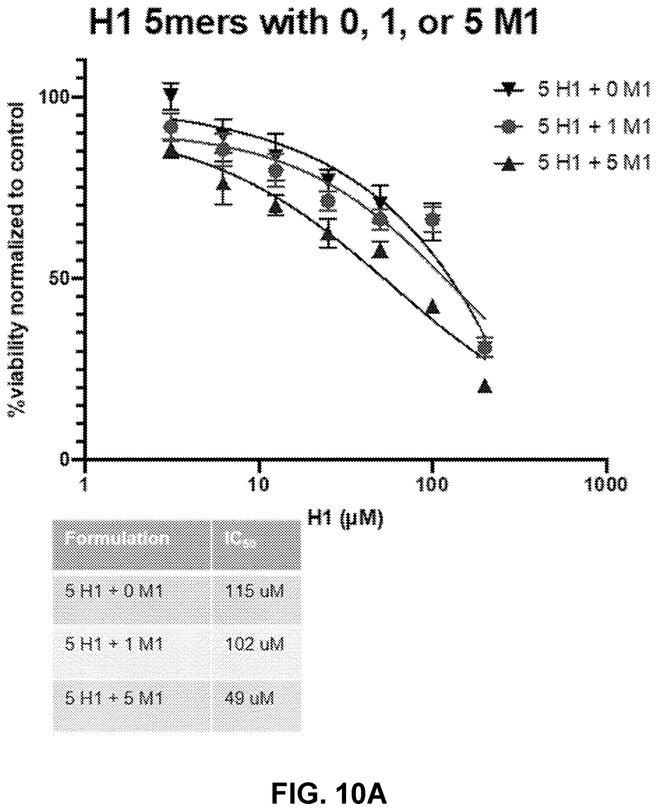

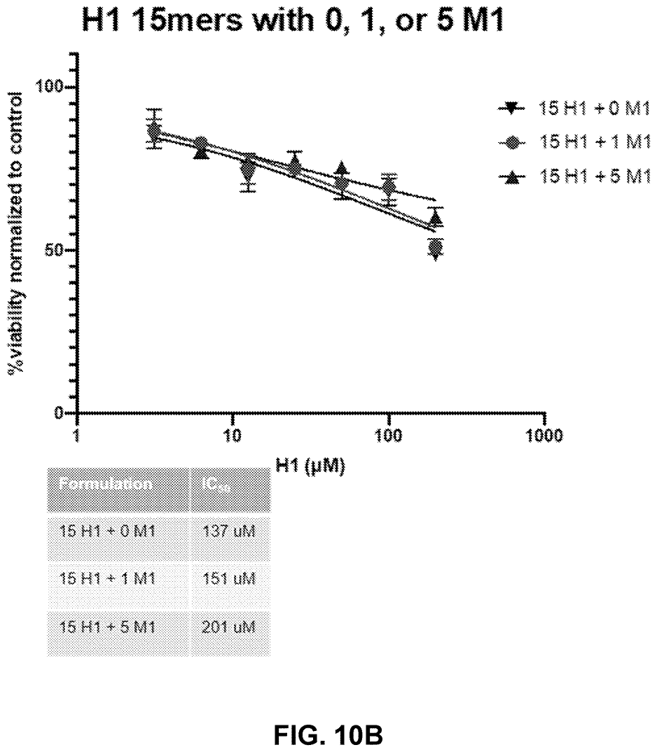

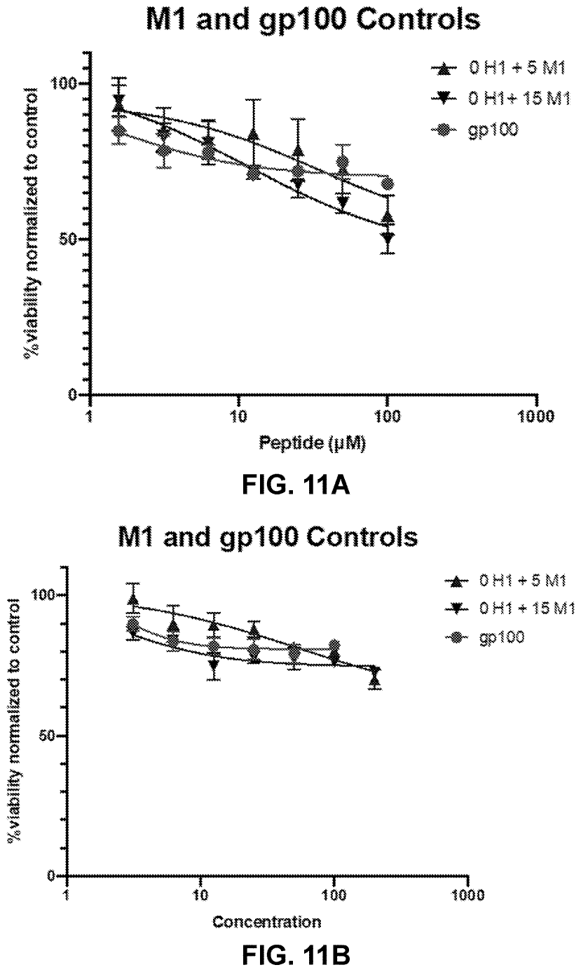

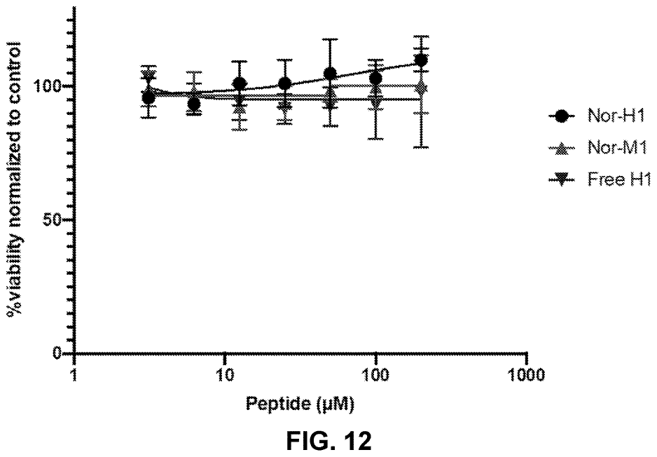

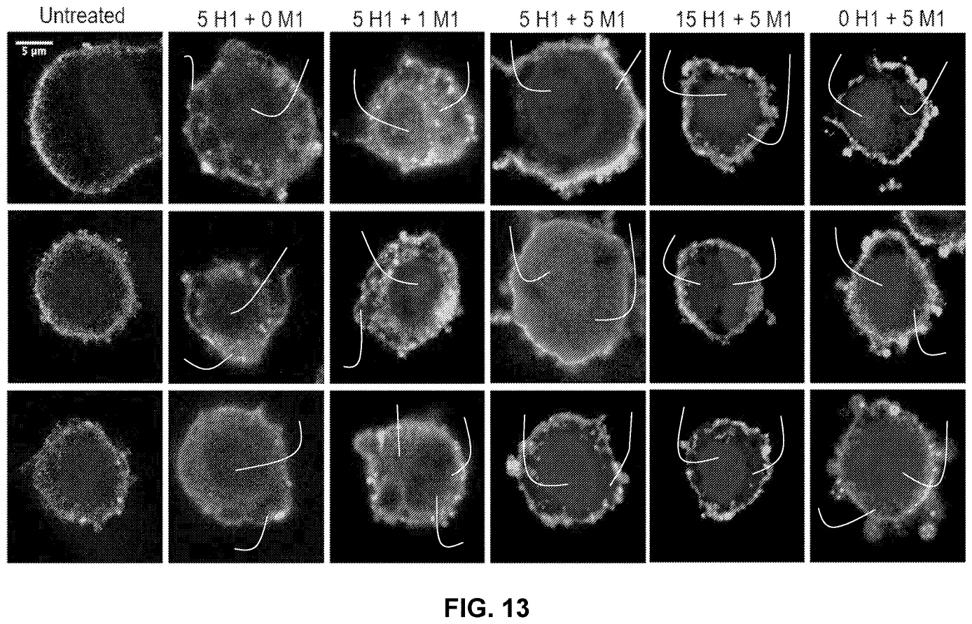

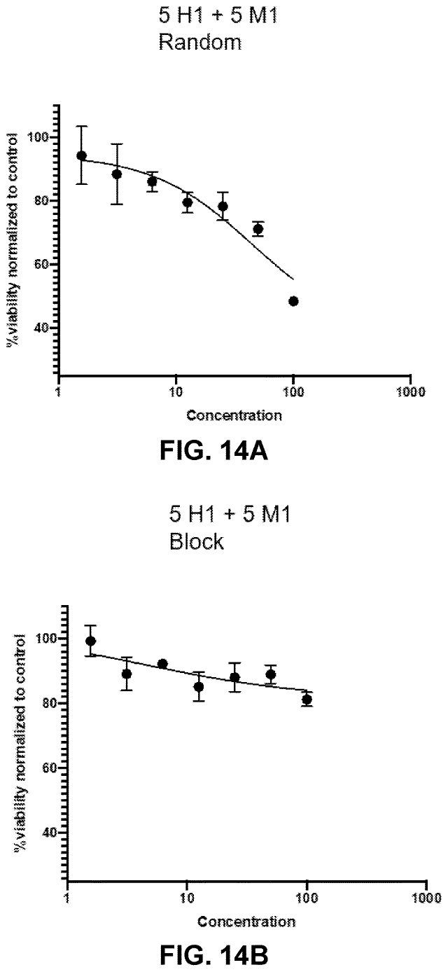

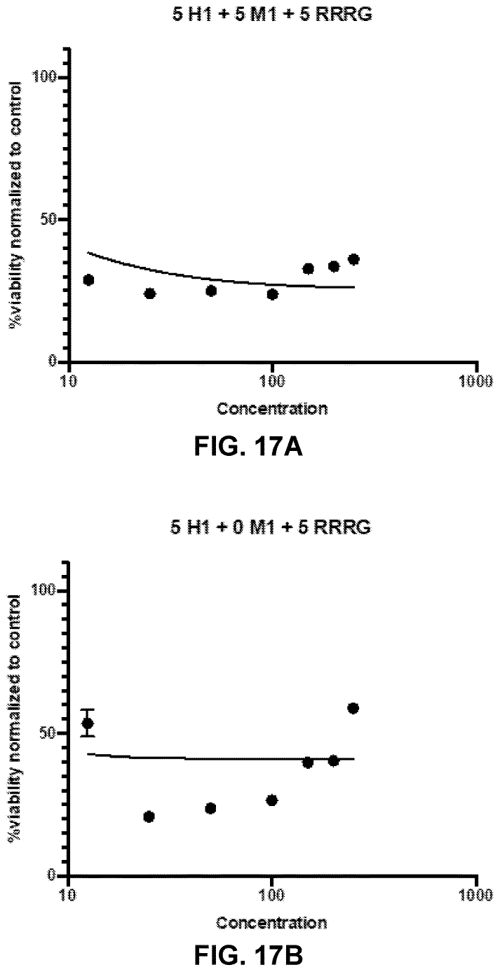

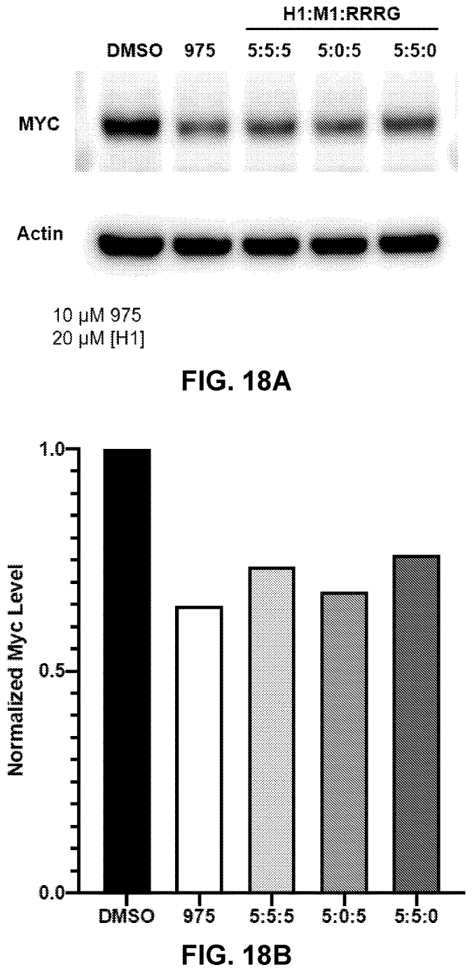

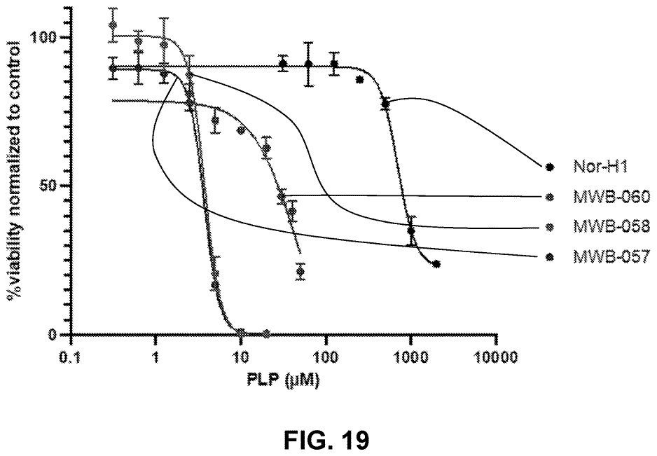

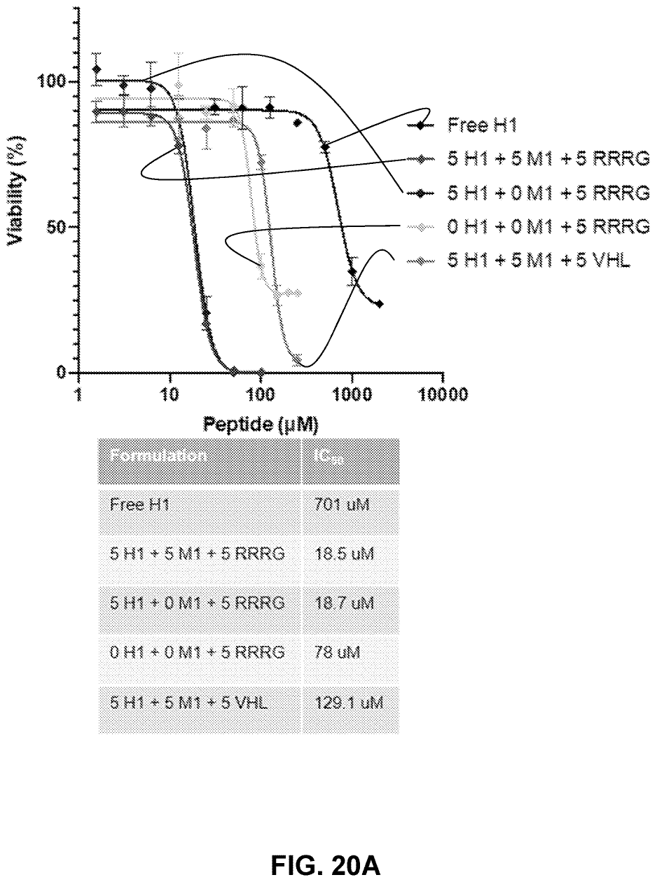

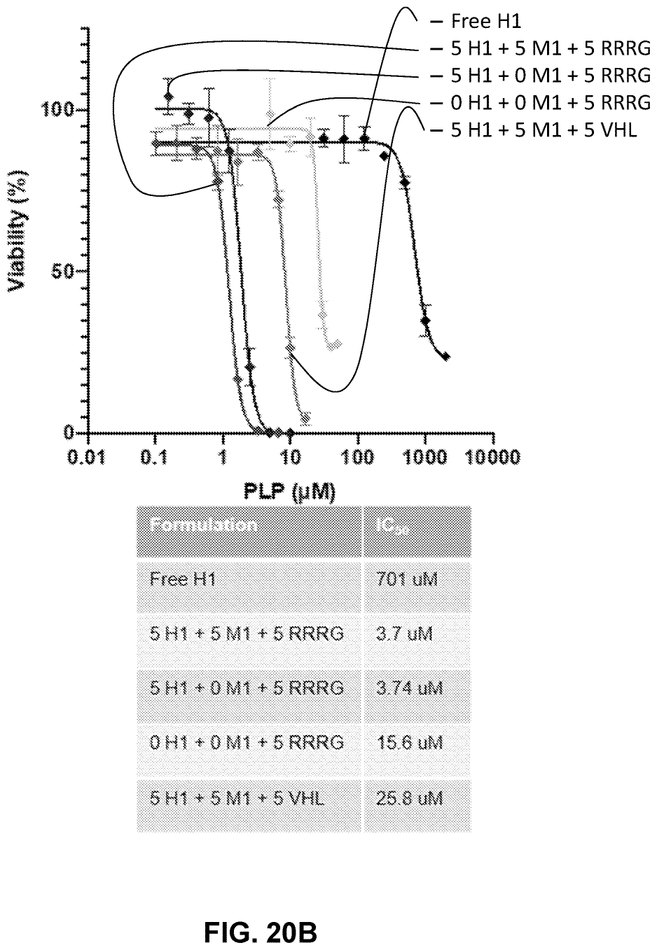

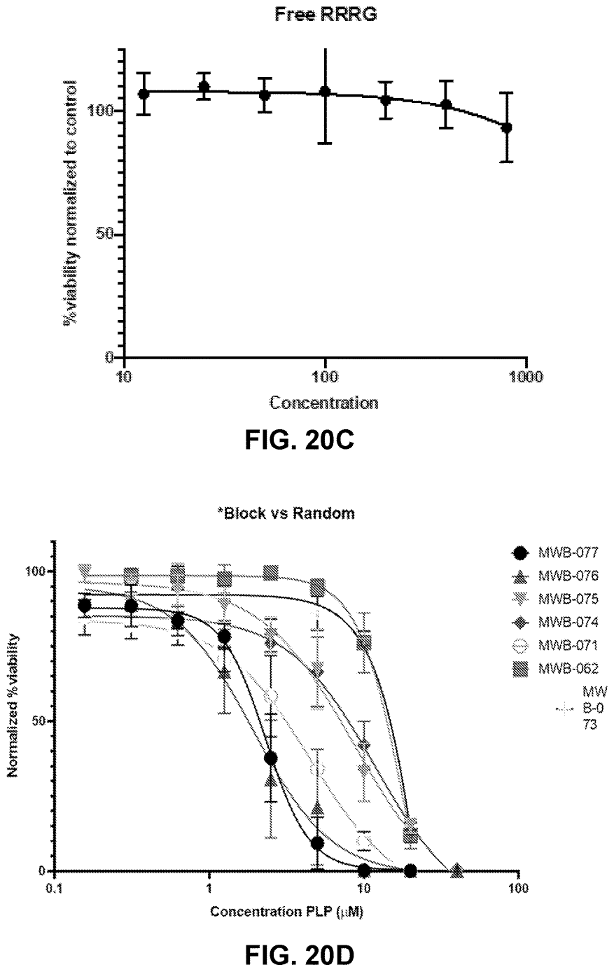

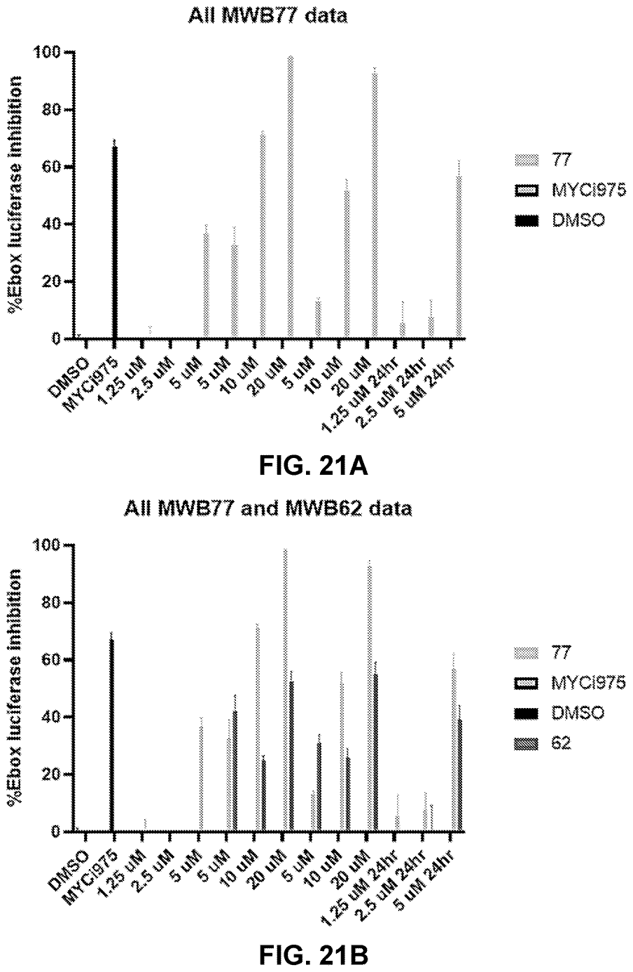

This example provides supplementary support for one or more of Examples 1-12, above. A test comparing the characterizations of H1-M1 PLPs (FIG. 9A) with H1 PLP homopolymers (FIG. 9B) was performed by GPC. FIGS. 10A-10B portrays the results from additional cell proliferation/toxicity evaluation experiments, including IC₅₀ values, normalized to control in order to characterize heterofunctional PLPs. This particular experiment focused on evaluating varying H1:M1 ratios (5:0, 5:1, and 1:1, FIG. 10A) and (15:0, 15:1, and 3:1, FIG. 10B) impact on viability. PC-3 cellular toxicity data from two independent experiments (1: FIG. 11A and 2: FIG. 11B) which were performed in order to establish little to no impact on PC-3 viability after treatment with M1 homopolymers and peptide controls (gp100). FIG. 12: An additional set cellular proliferation data to evaluate free peptide (Free HI) and monomer (Nor-H1 & Nor-M1) controls. In order to confirm cell and nuclear uptake, another set of experiments was run after a 1 hour incubation with the indicated PLP composition in the confocal microscopy images (FIG. 13). FIG. 14A-14B provides supporting data providing a comparison of the relative cellular toxicity between random H1-MI copolymers (FIG. 14A) and block H1-M1 copolymers (FIG. 14B). Tests evaluating the incorporation of a nuclear localization sequence (“M1”) into the platform were performed, and FIGS. 17A-17B demonstrated cellular proliferation data which suggested a possible hook effect with H1-M1-RRRG (FIG. 17A) and H1-RRRG (FIG. 17B) PLPs. As discussed in the previous examples, the ability of various PLPs were evaluated for their ability to bind Myc protein in vitro, with supplementary results found in: FIGS. 18A-18B: Western blot results (FIG. 18A) from pull-down biotin assay after treatment with 10 μM of a small molecule Myc inhibitor (975) and 20 μM of varying ratios of H1-M1-RRRG PLPs and a summary of the data normalized to Myc level (FIG. 18B). Further, as discussed in the above examples, many experiments were performed evaluating various PLP compositions impact on PC-3 viability. Supplementary data is provided in: FIG. 19, which depicts PC-3 viability data comparing copolymers, 5H1+5M1, 5H1+5RRRG, 5H1+5M1+5RRRG, as well as H1 monomer; FIGS. 20A-20D, which provides direct PC-3 viability data, including IC₅₀ values, from comparison of RRRG copolymers and VHL copolymers, including unpolymerized peptides (FIG. 20A), polymerized PLPs (FIG. 20B), and free RRRG normalized to control (FIG. 20C), and FIG. 20D provides a summary of viability data from two biological replicates comparing random/statistical (stat) PLP compositions and block PLP compositions; FIGS. 21A-21B displays the summary of results from Ebox luciferase inhibition assays evaluating HYDRAC PLP composition candidates (RRRG) for optimization; and FIG. 22 provides supporting CD spectrum results characterizing a H1-RRRG PLP, tested at increasing concentrations of the PLP, by evaluating ellipticity.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX—YY” (wherein XX and YY refer to claim numbers) is intended to provide a multiple dependent claim in the alternative form, and in some embodiments is interchangeable with the expression “as in any one of claims XX—YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every system, composition, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirely to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A polymer comprising:
a first repeating unit comprising a first polymer backbone group directly or indirectly covalently linked to a first functional sidechain comprising a Myc binding peptide.

2. A polymer comprising:
a first repeating unit comprising a first polymer backbone group directly or indirectly covalently linked to a first functional sidechain comprising a Myc binding peptide; and
a second repeating unit comprising a second polymer backbone group directly or indirectly covalently linked to a second functional sidechain comprising a degrader agent.

3. A polymer comprising:
a first repeating unit comprising a first polymer backbone group directly or indirectly covalently linked to a first functional sidechain comprising a Myc binding peptide;
a second repeating unit comprising a second polymer backbone group directly or indirectly covalently linked to a second functional sidechain comprising a degrader agent; and
a third repeating unit comprising a third polymer backbone group directly or indirectly covalently linked to a third functional sidechain comprising a nuclear localization peptide.

4. The polymer of any one of claims 1-3, wherein the polymer is of formula (FX1): 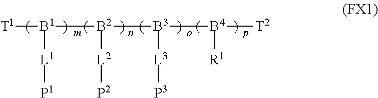
wherein:
T¹ and T² are each independently polymer backbone terminating groups that can be the same or different;
B¹, B², B³, and B⁴ are each independently polymer backbone subunits;
each L¹, L², and L³ is optionally present and each is independently a linking group;
each P¹, P², and P³ independently comprises a peptide or a small molecule;
at least one P¹ independently comprises a sequence having at least 75% or greater sequence identity of a Myc binding peptide;
at least one P² independently comprises a sequence having at least 75% or greater sequence identity of a degrader agent;
at least one P³ independently comprises a sequence having at least 75% or greater sequence identity of a nuclear localization peptide;
each R¹ is independently a substituent;
m is an integer selected from the range of 2 to 1000 (e.g., 2 to 500, 2 to 250, 2 to 100, 2 to 50, 2 to 20, 2 to 15, 2 to 10, or 2 to 5);
n is an integer selected from the range of 0 to 1000 (e.g., 2 to 500, 2 to 250, 2 to 100, 2 to 50, 2 to 20, 2 to 15, 2 to 10, or 2 to 5);
o is an integer selected from the range of 0 to 1000 (e.g., 2 to 250, 2 to 100, 2 to 50, 2 to 20, 2 to 15, 2 to 10, or 2 to 5);
p is an integer selected from the range of 0 to 1000 (e.g., 2 to 100, 2 to 50, 2 to 20, 2 to 15, 2 to 10, or 2 to 5);
each connecting line in formula (FX1) represents a covalent linkage comprising at least one of a single bond, a double bond, one or more atoms, or any combination thereof, optionally, for example, each connecting line represents a single bond or double bond;
each instance of B¹, B², B³, B⁴, L¹, L², L³, R¹, P¹, P², and P³ is the same as or different from any other instance of B¹, B², B³, B⁴, L¹, L², L³, R¹, P¹, P², and P³, respectively; and
the polymer is a block copolymer or a statistical copolymer.

5. The polymer of claim 4, wherein the polymer is of formula (FX2): 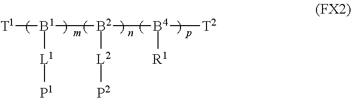
wherein:
T¹ and T² are each independently polymer backbone terminating groups that can be the same or different;
B¹, B², and B⁴ are each independently polymer backbone subunits;
each L¹ and L² is optionally present and each is independently a linking group;
each P¹ and P² independently comprise a peptide or a small molecule;
at least one P¹ independently comprises a sequence having at least 75% or greater sequence identity of a Myc binding peptide;
at least one P² independently comprises a sequence having at least 75% or greater sequence identity of a degrader agent;
each R¹ is independently a substituent;
m is an integer selected from the range of 2 to 1000 (e.g., 2 to 500, 2 to 250, 2 to 100, 2 to 50, 2 to 20, 2 to 15, 2 to 10, or 2 to 5);
n is an integer selected from the range of 0 to 1000 (e.g., 2 to 500, 2 to 250, 2 to 100, 2 to 50, 2 to 20, 2 to 15, 2 to 10, or 2 to 5);
p is an integer selected from the range of 0 to 1000 (e.g., 2 to 100, 2 to 50, 2 to 20, 2 to 15, 2 to 10, or 2 to 5);
each connecting line in formula (FX2) represents a covalent linkage comprising at least one of a single bond, a double bond, one or more atoms, or any combination thereof, optionally, for example, each connecting line represents a single bond or double bond;
each instance of B¹, B², B⁴, L¹, L², R¹, P¹, and P² is the same as or different from any other instance of B¹, B², B⁴, L¹, L², R¹, P¹, and P², respectively; and
the polymer is a block copolymer or a statistical copolymer.

6. The polymer of claim 4, wherein the polymer is of formula (FX3): 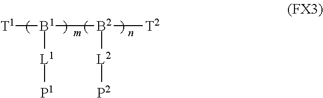
wherein:
T¹ and T² are each independently polymer backbone terminating groups that can be the same or different;
B¹ and B² are each independently polymer backbone subunits;
each L¹ and L² is optionally present and each is independently a linking group;
each P¹ and P² independently comprise a peptide or a small molecule;
at least one P¹ independently comprises a sequence having at least 75% or greater sequence identity of a Myc binding peptide;
at least one P² independently comprises a sequence having at least 75% or greater sequence identity of a degrader agent;
m is an integer selected from the range of 2 to 1000 (e.g., 2 to 500, 2 to 250, 2 to 100, 2 to 50, 2 to 20, 2 to 15, 2 to 10, or 2 to 5);
n is an integer selected from the range of 0 to 1000 (e.g., 2 to 500, 2 to 250, 2 to 100, 2 to 50, 2 to 20, 2 to 15, 2 to 10, or 2 to 5);
each connecting line in formula (FX3) represents a covalent linkage comprising at least one of a single bond, a double bond, one or more atoms, or any combination thereof, optionally, for example, each connecting line represents a single bond or double bond;
each instance of B¹, B², L¹, L², P¹, and P² is the same as or different from any other instance of B¹, B², L¹, L², P¹, and P², respectively; and
the polymer is a block copolymer or a statistical copolymer.

7. The polymer of any one of claims 4-6, wherein at least one of B¹, B², B³, or B⁴ independently comprises a polymerized monomer comprising an unsaturated monomer.

8. The polymer of claim 7, wherein the unsaturated monomer comprises an ethylenically unsaturated monomer, a norbomene monomer, or a norbomene dicarboxyimide.

9. The polymer of any one of claims 4-8, wherein each instance of a repeating unit (RU1), (RU2), and (RU3): 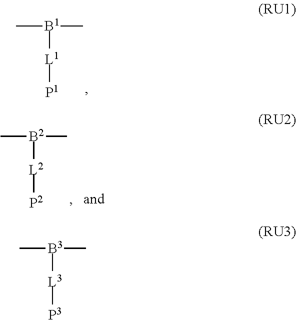
in formula (FX1), (FX2), or (FX3) is independently characterized by a repeating unit (RU4), (RU5), (RU6), or (RU7): 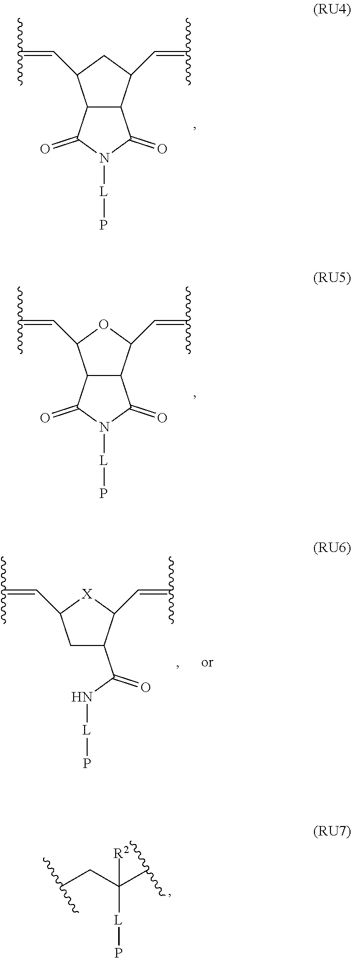
wherein:
L is optionally present and is L¹, L², or L³;
P is P¹, P², or P³;
R² is H or C₁-C₃ alkyl; and
X is CH₂ or O.

10. The polymer of claim 9, wherein each instance of the repeating unit (RU4): 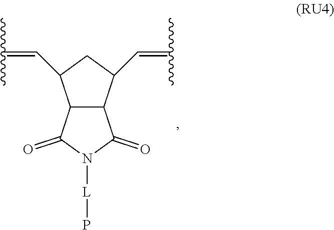
in formula (FX1), (FX2), or (FX3) is independently characterized by a repeating unit (RU4a), (RU4b), or (RU4c): 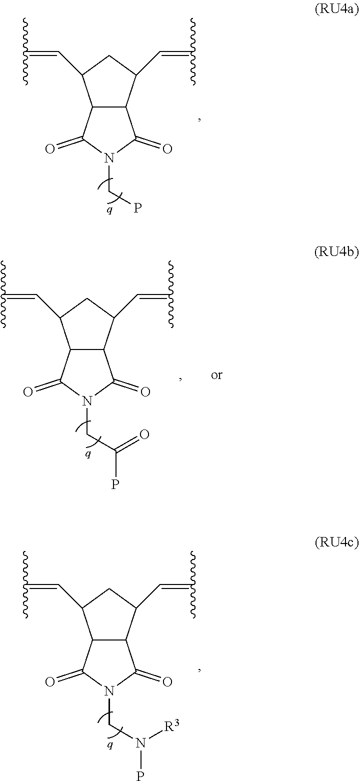
wherein q is an integer from 1 to 20 and R³ is a hydrogen or a C₁-C₅ alkyl.

11. The polymer of any one of claims 4-10, wherein each of Li, L², and L³, is independently selected from a single bond, an oxygen, and groups having an alkylene group, a heteroalkylene group, an alkenylene group, an arylene group, an alkoxy group, an acyl group, a triazole group, a diazole group, a pyrazole group, and combinations thereof.

12. The polymer of any one of claims 4-10, wherein each of Li, L², and L³, is independently selected from a single bond, —O—, C₁-C₁₀ alkyl, C₂-C₁₀ alkylene, C₁-C₁₀ heteroalkylene, C₃-C₁₀ arylene, C₁-C₁₀ alkoxy, C₁-C₁₀ acyl and combinations thereof.

13. The polymer of any one of claims 4-12, wherein each of Li, L², and L³, is independently selected from —(CH₂)ₙNR—, —(CH₂)ₙC(O)NR—, —(CH₂)ₙNRC(O)—, —(CH₂)ₙC(O)—and —(CH₂)ₙ—, wherein n is an integer from 1 to 20 and R is hydrogen or a C₁-C₅ alkyl.

14. The polymer of any one of claims 4-13, wherein each of R¹, T₁, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁—C₃₀ hydroxyl, C₁-C₃₀ alkoxy, C₂-C₃₀ alkenyl, C₂-C₃₀ alkynyl, C₅-C₃₀ alkylaryl, —CO₂R⁴, —CONR⁵R⁶, —COR⁷, —SORB, —OSR⁹, —SO₂R¹⁰, —OR¹¹, —SR¹², —NR¹³R¹⁴, —NR¹⁵COR¹⁶, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R⁴-R¹⁶ independently is H, C₅-C₁₀ aryl, or C₁-C₁₀ alkyl.

15. The polymer of any one of claims 4-14, wherein at least one of R¹, T¹, and T² independently further comprises an analytical tag.

16. The polymer of claim 15, wherein the analytical tag comprises an affinity tag, a solubilization tag, a chromatography tag, an epitope tag, or a fluorescence tag.

17. The polymer of any one of claims 4-16, wherein at least one of T¹ and T² comprises a degrader agent.

18. The polymer of any one of claims 4-17, wherein at least one of T¹ and T² comprises a nuclear localization peptide.

19. The polymer of any one of claims 4-18, wherein at least one of R¹, T¹, and T² independently comprises a cleavable linker.

20. The polymer of claim 19, wherein the cleavable linker is a disulfide linker, a hydrazone, or other suitable acid-labile linker.

21. The polymer of claim 19, wherein the cleavable linker is a p-glucuronide linker, a peptide linker, or other suitable enzymatically cleavable linker.

22. The polymer of any one of claims 1-21, wherein the polymer is characterized by a number average molecular weight of 1 kDa to 500 kDa.

23. The polymer of any one of claims 1-21, wherein the polymer is characterized by a number average molecular weight of less than or equal to 50 kDa.

24. The polymer of any one of claims 1-21, wherein the polymer is characterized by a number average molecular weight of less than or equal to 25 kDa.

25. The polymer of any one of claims 1-24, wherein the polymer has an average degree of polymerization from 2 to 1000.

26. The polymer of any one of claims 1-24, wherein the polymer has an average degree of polymerization of 2 to 100.

27. The polymer of any one of claims 1-24, wherein the polymer has an average degree of polymerization of 2 to 50.

28. The polymer of any one of claims 1-24, wherein the polymer has an average degree of polymerization of 2 to 25.

29. The polymer of any one of claims 1-28, wherein the polymer has an average length of less than or equal to 100 nm.

30. The polymer of any one of claims 1-28, wherein the polymer has an average length of less than or equal to 20 nm.

31. The polymer of any one of claims 1-30, wherein the polymer is characterized by an average first repeating unit to second repeating unit ratio of between 1:1 and 15:1.

32. The polymer of any one of claims 1-30, wherein the polymer is characterized by an average first repeating unit to second repeating unit ratio of between 1:1 and 5:1.

33. The polymer of any one of claims 1-30, wherein the polymer is characterized by an average first repeating unit to second repeating unit ratio of between 1:1 and 1:15.

34. The polymer of any one of claims 1-30, wherein the polymer is characterized by proportionate amounts of the first repeating unit, the second repeating unit, and the third repeating unit.

35. The polymer of any one of claims 1-30, wherein the polymer comprises from 50-75% of the first repeating unit, from 10-30% of the second repeating unit, and from 10-30% of the third repeating unit.

36. The polymer of any one of claims 1-35, wherein the polymer is prepared by a living polymerization method optionally selected from ring-opening metathesis polymerization (ROMP), reversible addition-fragmentation chain transfer polymerization (RAFT), or atom transfer radical polymerization (ATRP).

37. The polymer of any one of claims 1-36, wherein the polymer is characterized by a high-density brush copolymer having a brush density greater than or equal to 75%.

38. The polymer of any one of claims 1-36, wherein the polymer is characterized by a high-density brush copolymer having a brush density greater than or equal to 85%.

39. The polymer of any one of claims 1-36, wherein the polymer is characterized by a high-density brush copolymer having a brush density greater than or equal to 95%.

40. The polymer of any one of claims 1-36, wherein the polymer is characterized by a high-density brush copolymer having a brush density greater than or equal to 99%.

41. The polymer of any one of claims 1-40, wherein at least one functional sidechain comprises a spacer sequence having between 3 and 15 amino acids.

42. The polymer of any one of claims 1-41, wherein the first repeating unit is characterized by a number average molecular weight of 1 kDa to 20 kDa.

43. The polymer of any one of claims 1-41, wherein the first repeating unit is characterized by a number average molecular weight of less than or equal to 10 kDa.

44. The polymer of any one of claims 1-41, wherein the first repeating unit is characterized by a number average molecular weight of less than or equal to 5 kDa.

45. The polymer of any one of claims 2-44, wherein the second repeating unit is characterized by a number average molecular weight of 1 kDa to 10 kDa.

46. The polymer of any one of claims 2-44, wherein the second repeating unit is characterized by a number average molecular weight of less than or equal to 5 kDa.

47. The polymer of any one of claims 2-44, wherein the second repeating unit is characterized by a number average molecular weight of less than or equal to 2.5 kDa.

48. The polymer of any one of claims 3-47, wherein the third repeating unit is characterized by a number average molecular weight of 1 kDa to 20 kDa.

49. The polymer of any one of claims 3-47, wherein the third repeating unit is characterized by a number average molecular weight of less than or equal to 10 kDa.

50. The polymer of any one of claims 3-47, wherein the third repeating unit is characterized by a number average molecular weight of less than or equal to 5 kDa.

51. The polymer of claim 1 further characterized by a number average molecular weight of 1 kDa to 30 kDa.

52. The polymer of claim 1 further characterized by a number average molecular weight of less than or equal to 20 kDa.

53. The polymer of claim 1 further characterized by a number average molecular weight of less than or equal to 15 kDa.

54. The polymer of claim 2 further characterized by a number average molecular weight of 1 kDa to 40 kDa.

55. The polymer of claim 2 further characterized by a number average molecular weight of less than or equal to 30 kDa.

56. The polymer of claim 2 further characterized by a number average molecular weight of less than or equal to 25 kDa.

57. The polymer of any one of claims 4-56, wherein 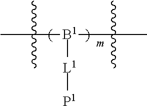
is characterized by a P¹ peptide density of greater than or equal to 80%.

58. The polymer of any one of claims 4-56, wherein 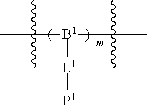
is characterized by a P¹ peptide density of greater than or equal to 90%.

59. The polymer of any one of claims 4-56, wherein 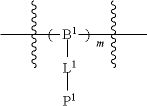
is characterized by a P¹ peptide density of greater than or equal to 95%.

60. The polymer of any one of claims 4-56, wherein 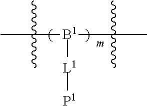
is characterized by a P¹ peptide density of greater than or equal to 99%.

61. The polymer of any one of claims 4-60, wherein 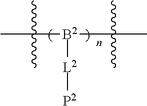
is characterized by a P² peptide density of greater than or equal to 80%.

62. The polymer of any one of claims 4-60, wherein 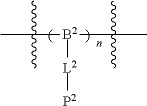
is characterized by a P² peptide density of greater than or equal to 90%.

63. The polymer of any one of claims 4-60, wherein 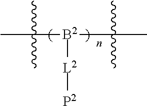
is characterized by a P² peptide density of greater than or equal to 95%.

64. The polymer of any one of claims 4-60, wherein 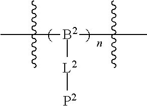
is characterized by a P² peptide density of greater than or equal to 99%.

65. The polymer of any one of claims 4-64, wherein 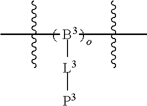
is characterized by a P³ peptide density of greater than or equal to 80%.

66. The polymer of any one of claims 4-64, wherein 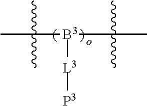
is characterized by a P³ peptide density of greater than or equal to 90%.

67. The polymer of any one of claims 4-64, wherein 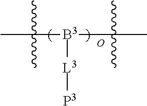
is characterized by a P³ peptide density of greater than or equal to 95%.

68. The polymer of any one of claims 4-64, wherein 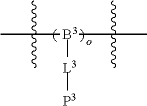
is characterized by a P³ peptide density of greater than or equal to 99%.

69. The polymer of any one of claims 4-68, wherein each P¹ independently comprises from 3 to 100 amino acid residues.

70. The polymer of any one of claims 4-69, wherein at least one P¹ further comprises a therapeutic small molecule.

71. The polymer of claim 70, wherein the therapeutic small molecule is a Myc inhibitor.

72. The polymer of any one of claims 4-71, wherein at least one P¹ comprises an amino acid sequence derived from the amino acid sequence of c-Myc.

73. The polymer of any one of claims 4-72, wherein at least one P¹ comprises an amino acid sequence derived from the amino acid sequence of Max.

74. The polymer of any one of claims 4-73, wherein at least one P¹ comprises a competitive inhibitor peptide.

75. The polymer of any one of claims 4-74, wherein at least one P¹ comprises a c-Myc inhibitor peptide.

76. The polymer of any one of claims 4-75, wherein at least one P¹ is characterized by a helical structure.

77. The polymer of any one of claims 4-76, wherein at least one P¹ comprises a sequence having 75% or greater sequence identity of SEQ ID NO: 1 (NELKRAFAALRDQI).

78. The polymer of any one of claims 4-77, wherein at least one P¹ comprises a sequence having 75% or greater sequence identity of SEQ ID NO: 2 (NELKRSFFALRDQI).

79. The polymer of any one of claims 4-78, wherein at least one P¹ comprises a sequence having 75% or greater sequence identity of SEQ ID NO: 3 (NELKRSFAALRDQI).

80. The polymer of any one of claims 4-79, wherein at least one P¹ comprises a sequence having 75% or greater sequence identity of SEQ ID NO: 4 (VQAEEQKLISEEDLLRKRREQLKHKLEQLRN).

81. The polymer of any one of claims 4-80, wherein at least one P¹ comprises a sequence having 75% or greater sequence identity of SEQ ID NO: 5 (AEEQKLISEEDLLRKRREQLKHKLEQLRNSC).

82. The polymer of any one of claims 4-81, wherein at least one P¹ comprises a sequence having 75% or greater sequence identity of SEQ ID NO: 6 (PGHLKGREIGLWYAKKQGQKNK).

83. The polymer of any one of claims 4-82, wherein at least one P¹ comprises a sequence having 75% or greater sequence identity of SEQ ID NO: 7 (HQQDIDDLKRQNALLEQQVRAL).

84. The polymer of any one of claims 4-83, wherein at least one P¹ comprises a sequence having 75% or greater sequence identity of SEQ ID NO: 8 (HHNALERKRRDHIKDSFHSLRDS).

85. The polymer of any one of claims 4-84, wherein at least one P² comprises a catalyst.

86. The polymer of any one of claims 4-85, wherein at least one P² comprises a proteasome recruiter.

87. The polymer of any one of claims 4-86, wherein at least one P² comprises a peptide capable of binding to an E3 ligase.

88. The polymer of any one of claims 4-87, wherein each P² independently comprises from 3 to 100 amino acid residues.

89. The polymer of any one of claims 4-88, wherein at least one P² comprises a small molecule degrader.

90. The polymer of claim 89, wherein the small molecule degrader comprises a thalidomide.

91. The polymer of any one of claims 4-90, wherein at least one P² comprises a sequence having 75% or greater sequence identity of SEQ ID NO: 9 (RRRG).

92. The polymer of any one of claims 4-91, wherein at least one P² comprises a sequence having 75% or greater sequence identity of SEQ ID NO: 10 (RRRGN).

93. The polymer of any one of claims 4-92, wherein at least one P² comprises a sequence having 75% or greater sequence identity of SEQ ID NO: 11 (TRGVEEVAEGVVLLRRRG).

94. The polymer of any one of claims 4-93, wherein at least one P² comprises a sequence having 75% or greater sequence identity of SEQ ID NO: 12 (TRGVEEVAEGVVLLRRRGN).

95. The polymer of any one of claims 4-94, wherein at least one P² comprises a sequence having 75% or greater sequence identity of SEQ ID NO: 13 (RQRAIDLFKANELA).

96. The polymer of any one of claims 4-95, wherein at least one P² comprises a sequence having 75% or greater sequence identity of SEQ ID NO: 14 (ALAPYIP).

97. The polymer of any one of claims 4-96, wherein at least one P² comprises a sequence having 75% or greater sequence identity of SEQ ID NO: 15 (ALAPYIPR).

98. The polymer of any one of claims 4-97, wherein at least one P² comprises a sequence having 75% or greater sequence identity of SEQ ID NO: 16 (LDPETGEYL).

99. The polymer of any one of claims 4-98, wherein at least one P² comprises a sequence having 75% or greater sequence identity of SEQ ID NO: 17 (DRHDSGLDSM).

100. The polymer of any one of claims 4-99, wherein each P³ independently comprises from 3 to 100 amino acid residues.

101. The polymer of any one of claims 4-100, wherein at least one P³ is characterized by a net positive charge.

102. The polymer of any one of claims 4-101, wherein at least one P³ further comprises a charge modulating domain.

103. The polymer of claim 102, wherein the charge modulating domain is a cationic residue domain.

104. The polymer of claim 103, wherein the cationic residue domain consists of lysine, arginine, histidine, or a combination thereof.

105. The polymer of any one of claims 4-104, wherein at least one P³ comprises a sequence having 75% or greater sequence identity of SEQ ID NO: 18 (PAAKRVKLD).

106. The polymer of any one of claims 4-105, wherein at least one P³ comprises a sequence having 75% or greater sequence identity of SEQ ID NO: 19 (PKLKRQ).

107. The polymer of any one of claims 4-106, wherein at least one P³ comprises a sequence having 75% or greater sequence identity of SEQ ID NO: 20 (RPRK).

108. The polymer of any one of claims 4-107, wherein at least one P³ comprises a sequence having 75% or greater sequence identity of SEQ ID NO: 21 (RRARRPRG).

109. The polymer of any one of claims 4-108, wherein at least one P³ comprises a sequence having 75% or greater sequence identity of SEQ ID NO: 22 (GKRKLITSEEERSPAKRGRKS).

110. The polymer of any one of claims 4-109, wherein at least one P³ comprises a sequence having 75% or greater sequence identity of SEQ ID NO: 23 (KGKKGRTQKEKKAARARSKGKN)

111. The polymer of any one of claims 4-110, wherein at least one P³ comprises a sequence having 75% or greater sequence identity of SEQ ID NO: 24 (RKRCAAGVGGGPAGCPAPGSTPLKKPRR).

112. The polymer of any one of claims 4-111, wherein at least one P³ comprises a sequence having 75% or greater sequence identity of SEQ ID NO: 25 (RKPVTAQERQREREEKRRRRQERAKEREKRRQERER).

113. The polymer of any one of claims 4-112, wherein at least one P³ comprises a sequence having 75% or greater sequence identity of SEQ ID NO: 26 (RSGGNHRRNGRGGRGGYNRRNNGYHPY).

114. The polymer of any one of claims 4-113, wherein at least one P³ comprises a sequence having 75% or greater sequence identity of SEQ ID NO: 27 (TLLLRETMNNLGVSDHAVLSRKTPQPY).

115. The polymer of any one of claims 4-114, wherein at least one P³ comprises a sequence having 75% or greater sequence identity of SEQ ID NO: 28 (PGKMDKGEHRQERRDRPY).

116. The polymer of any one of claims 4-115, wherein at least one P³ comprises a sequence having 75% or greater sequence identity of SEQ ID NO: 29 (GKKKKGKPGKRREQRKKKRRT).

117. The polymer of any one of claims 4-116, wherein at least one P³ comprises a sequence having 75% or greater sequence identity of SEQ ID NO: 30 (SANKVTKNKSNSSPYLNKRKGKPGPDS).

118. The polymer of any one of claims 4-117, wherein at least one P³ comprises a sequence having 75% or greater sequence identity of SEQ ID NO: 31 (VHSHKKKKIPTSPTFTTPKTLTLRRQPKYPRKSAPRRNKLDHY).

119. The polymer of any one of claims 4-118, wherein at least one P³ comprises a sequence having 75% or greater sequence identity of SEQ ID NO: 32 (RKHKTNRKPR).

120. The polymer of any one of claims 4-119, wherein at least one P³ comprises a sequence having 75% or greater sequence identity of SEQ ID NO: 33 (NRRAKAKR).

121. The polymer of any one of claims 4-120, wherein at least one P³ comprises a sequence having 75% or greater sequence identity of SEQ ID NO: 34 (RNKKKK).

122. The polymer of any one of claims 4-121, wherein at least one P³ comprises a sequence having 75% or greater sequence identity of SEQ ID NO: 35 (RKVIK).

123. The polymer of any one of claims 4-122, wherein at least one P³ comprises a spacer sequence having between 3 and 15 amino acids.

124. A pharmaceutical composition comprising the polymer of any one of claims 1-123 and a pharmaceutically acceptable excipient.

125. A method of suppressing transcriptional expression of a target gene in a cell comprising:
contacting the cell with an effective amount of the polymer of any one of claims 1-123 or the pharmaceutical composition of claim 124;
wherein the contacting results in the suppressing transcriptional expression of the target gene in the cell.

126. The method of claim 125, wherein the target gene is activated by a Myc/Max heterodimer.

127. The method of claim 125, wherein the contacting results in a disruption of a DNA-protein interaction.

128. The method of claim 125, wherein the contacting results in a disruption of a protein-protein interaction.

129. The method of claim 128, wherein the protein-protein interaction comprises a formation of a Myc/Max dimer.

130. The method of claim 129, wherein the contacting results in a recruitment of a proteasome.

131. The method of any one of claims 125-130, wherein the cell is a eukaryotic cell.

132. The method of claim 125, wherein transcriptional expression of more than one gene is suppressed.

133. The method of any one of claims 125-132, wherein the target gene is a pro-proliferative gene.

134. A method of targeting a nuclear localized protein in a cell comprising:
introducing the polymer of any one of claims 1-123 or the pharmaceutical composition of claim 124 to a cell;
wherein the introducing results in at least a portion of the polymer or the pharmaceutical composition to bind with at least a portion of the nuclear localized protein;
thereby targeting the nuclear localized protein in a cell.

135. The method of claim 134, wherein the nuclear localized protein is a c-Myc protein.

136. The method of claim 135, wherein the introducing results in at least a portion of the polymer or the pharmaceutical composition to bind with at least a portion of the N-terminal region of the c-Myc protein.

137. The method of claim 135, wherein the introducing results in at least a portion of the polymer or the pharmaceutical composition to bind with at least a portion of the C-terminal region of the c-Myc protein.

138. The method of claim 135, wherein the introducing results in at least a portion of the polymer or the pharmaceutical composition to bind with at least a portion of the bHLHZip domain of the c-Myc protein.

139. A method of treating or managing a condition of a subject comprising:
administering to the subject a therapeutically effective amount of the polymer of any one of claims 1-123 or the pharmaceutical composition of claim 124;
wherein the administering results in the treating or managing of the condition of the subject.

140. The method of claim 139 further comprising:
repeating the step of administering to the subject the therapeutically effective amount of the polymer of any one of claims 1-123 or the pharmaceutical composition of claim 124.

141. The method of claim 139 or claim 140, wherein the method results in an accumulation of proteasomes at the disease site.

142. The method of any one of claims 139-141, wherein the method results in an accumulation of a Myc binding peptide in a nucleus of a cell.

143. The method of any one of claims 139-141, wherein the administering to the subject comprises intravenous administration, subcutaneous administration, intramuscular administration, topical administration, oral administration, or a combination thereof.

144. The method of any one of claims 139-141, wherein the condition is a Myc-dependent cancer.

145. The method of claim 144, wherein the method interrupts the protein-protein interaction between c-Myc and Max.

146. The method of claim 144, wherein the method interrupts the DNA-protein interaction between DNA and c-Myc.

147. The method of claim 144, wherein the method inhibits transcriptional activity of c-Myc.

148. The method of any one of claims 139-141, wherein the condition is a respiratory disease.

149. The method of claim 148, wherein the respiratory disease is COPD, asthma, emphysema, potential treatment for smokers, idiopathic pulmonary fibrosis, chronic sarcoidosis, or hypersensitivity pneumonitis.

150. The method of any one of claims 139-141, wherein the condition is associated with an inflammatory state, increased oxidative stress, autoimmune pathophysiology, chemo-preventative measures, neurodegeneration, or a combination thereof.

151. The method of any one of claims 139-141, wherein the condition is a gastrointestinal disease.

152. The method of claim 151, wherein the gastrointestinal disease is ulcerative colitis, ulcers, prevent acetaminophen toxicity, non-alcoholic steatohepatitis, primary biliary cholangitis, cirrhosis, type 2 diabetes, diabetic nephropathy, or Crohn's disease.

153. The method of any one of claims 139-141, wherein the condition is an autoimmune disease.

154. The method of claim 153, wherein the autoimmune disease is multiple sclerosis, systemic lupus erythematous, Sjogren syndrome, rheumatoid arthritis, vitiligo, or psoriasis.

155. The method of any one of claims 139-141, wherein the condition is a genetic disease.

156. The method of claim 155, wherein the genetic disease is polycystic kidney disease.

157. The method of any one of claims 139-141, wherein the condition is an age-associated pathology.


SEQUENCE LISTING (35 sequences)

SEQ ID NO: 1 - protein, 14 aa, synthetic construct
NELKRAFAALRDQI

SEQ ID NO: 2 - protein, 14 aa, synthetic construct
NELKRSFFALRDQI

SEQ ID NO: 3 - protein, 14 aa, synthetic construct
NELKRSFAALRDQI

SEQ ID NO: 4 - protein, 31 aa, synthetic construct
VQAEEQKLISEEDLLRKRREQLKHKLEQLRN

SEQ ID NO: 5 - protein, 31 aa, synthetic construct
AEEQKLISEEDLLRKRREQLKHKLEQLRNSC

SEQ ID NO: 6 - protein, 22 aa, synthetic construct
PGHLKGREIGLWYAKKQGQKNK

SEQ ID NO: 7 - protein, 22 aa, synthetic construct
HQQDIDDLKRQNALLEQQVRAL

SEQ ID NO: 8 - protein, 23 aa, synthetic construct
HHNALERKRRDHIKDSFHSLRDS

SEQ ID NO: 9 - protein, 4 aa, synthetic construct
RRRG

SEQ ID NO: 10 - protein, 5 aa, synthetic construct
RRRGN

SEQ ID NO: 11 - protein, 18 aa, synthetic construct
TRGVEEVAEGVVLLRRRG

SEQ ID NO: 12 - protein, 19 aa, synthetic construct
TRGVEEVAEGVVLLRRRGN

SEQ ID NO: 13 - protein, 14 aa, synthetic construct
RQRAIDLFKANELA

SEQ ID NO: 14 - protein, 7 aa, synthetic construct
ALAPYIP

SEQ ID NO: 15 - protein, 8 aa, synthetic construct
ALAPYIPR

SEQ ID NO: 16 - protein, 9 aa, synthetic construct
LDPETGEYL

SEQ ID NO: 17 - protein, 10 aa, synthetic construct
DRHDSGLDSM

SEQ ID NO: 18 - protein, 9 aa, synthetic construct
PAAKRVKLD

SEQ ID NO: 19 - protein, 6 aa, synthetic construct
PKLKRQ

SEQ ID NO: 20 - protein, 4 aa, synthetic construct
RPRK

SEQ ID NO: 21 - protein, 8 aa, synthetic construct
RRARRPRG

SEQ ID NO: 22 - protein, 21 aa, synthetic construct
GKRKLITSEEERSPAKRGRKS

SEQ ID NO: 23 - protein, 22 aa, synthetic construct
KGKKGRTQKEKKAARARSKGKN

SEQ ID NO: 24 - protein, 28 aa, synthetic construct
RKRCAAGVGGGPAGCPAPGSTPLKKPRR

SEQ ID NO: 25 - protein, 36 aa, synthetic construct
RKPVTAQERQREREEKRRRRQERAKEREKRRQERER

SEQ ID NO: 26 - protein, 27 aa, synthetic construct
RSGGNHRRNGRGGRGGYNRRNNGYHPY

SEQ ID NO: 27 - protein, 27 aa, synthetic construct
TLLLRETMNNLGVSDHAVLSRKTPQPY

SEQ ID NO: 28 - protein, 18 aa, synthetic construct
PGKMDKGEHRQERRDRPY

SEQ ID NO: 29 - protein, 21 aa, synthetic construct
GKKKKGKPGKRREQRKKKRRT

SEQ ID NO: 30 - protein, 27 aa, synthetic construct
SANKVTKNKSNSSPYLNKRKGKPGPDS

SEQ ID NO: 31 - protein, 43 aa, synthetic construct
VHSHKKKKIPTSPTFTTPKTLTLRRQPKYPRKSAPRRNKLDHY

SEQ ID NO: 32 - protein, 10 aa, synthetic construct
RKHKTNRKPR

SEQ ID NO: 33 - protein, 8 aa, synthetic construct
NRRAKAKR

SEQ ID NO: 34 - protein, 6 aa, synthetic construct
RNKKKK

SEQ ID NO: 35 - protein, 5 aa, synthetic construct
RKVIK